(19) United States
(12) Patent Application Publication
(10) Pub. No.: US 2014/0058099 A1
(43) Pub. Date: Feb. 27, 2014

(54) NOVEL COMPOUND, CHARGE TRANSPORT MATERIAL, AND ORGANIC DEVICE

(71) Applicants: Atsushi Wakamiya, Uji-city (JP); Hidetaka Nishimura, Uji-city (JP); Yasujiro Murata, Uji-city (JP); Tatsuya Fukushima, Uji-city (JP); Hironori Kaji, Uji-city (JP)

(72) Inventors: Atsushi Wakamiya, Uji-city (JP); Hidetaka Nishimura, Uji-city (JP); Yasujiro Murata, Uji-city (JP); Tatsuya Fukushima, Uji-city (JP); Hironori Kaji, Uji-city (JP)

(73) Assignee: KYUSHU UNIVERSITY NATIONAL UNIVERSITY CORPORATION, Fukuoka-shi, Fukuoka (JP)

(21) Appl. No.: 14/002,947

(22) PCT Filed: Mar. 2, 2012
(86) PCT No.: PCT/JP2012/055287

§ 371 Date: Nov. 12, 2013

(30) Foreign Application Priority Data
Mar. 3, 2011 (JP) 2011-046888
Sep. 5, 2011 (JP) 2011-193294

(51) Int. Cl. H01L 51/00

(52) U.S. Cl. CPC H01L 51/0071; H01L 51/0072
USPC 544/35; 544/73; 546/102; 546/103; 546/105; 544/347; 568/3

(57) ABSTRACT

The compounds represented fey the following general formula is is thermally stable and has excellent characteristics as a charge transport material [Ar¹ represents a single bond, a benzene ring, etc.; X¹ represents a linking group that links via an oxygen atom, a sulfur atom, a carbon atom, a nitrogen atom, a phosphorus atom or a silicon atom; either one of L¹ and L², and L³ and L⁴ bond to each other to represent a linking group that links via an oxygen atom, a sulfur atom, a carbon atom, a nitrogen atom, a phosphorus atom or a silicon atom; the other of L¹ and L², and L³ and L⁴ represent a hydrogen atom or a substituent; Y¹ represents a linking group that links via a nitrogen atom, a boron atom or a phosphorus atom; R¹, R², R⁵ to R⁷ and R¹⁰ to R¹² represent a hydrogen atom or a substituent; and n1 indicates an integer of 2 or more.].

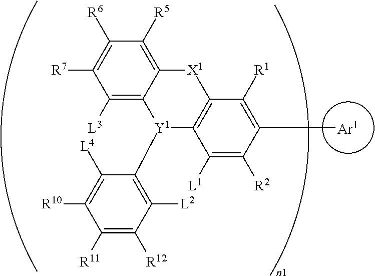

### TECHNICAL FIELD

The present invention relates to a novel compound and a charge transport material comprising the novel compound. The invention also relates to an organic device such as an organic electroluminescence element, an organic thin-film solar cell and the like using the novel compound.

### BACKGROUND ART

A charge transport material having a high charge mobility in needed for an organic device such as an organic electroluminescence element, an organic thin-film solar cell, etc. Various charge transport materials have heretofore been proposed, and in particular, compounds having a triphenylamine structure are known to have a relatively high charge mobility.

As compounds having a triphenylamine structure, for example, triphenylamine dimers such as N,N′-diphenyl-N,N′-bis(3-methylphenyl)-1,1′-biphenyl-4,4′-diamine [TPD] and N,N′-diphenyl-N,N′-bis(1-naphthyl)-1,1′-biphenyl-4,4′-diamine [α-NPD] each having the following structure have been widely known and have been put into practical use.

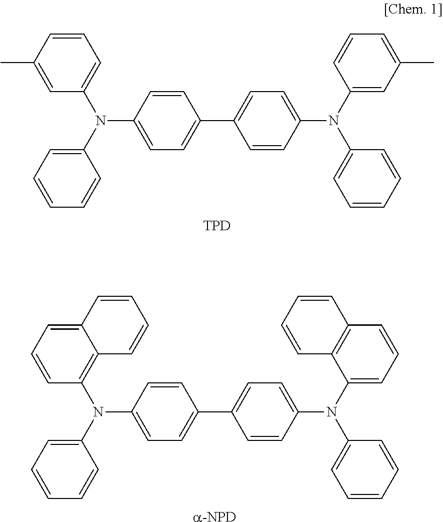

Also known are triphenylamine derivatives (monomer) characterized by linking the aromatic rings constituting the triphenylamine with a linking group to thereby enhance the planarity of the triphenylamine (see PTL 1). The triphenylamine derivatives are shown to be more excellent in hole transportability than TPD. However, the patent literature describes nothing relating to production or a dimer of a triphenylamine derivative.

### CITATION LIST

### Patent Literature

PTL 1: JP-A 11-339868

### SUMMARY OF INVENTION

### Technical Problem

As the charge transport material for use in organic devices such as organic electroluminescence elements, organic thin-film solar cells and the like, preferred are those having properties of such that their amorphous state is stable and they hardly crystallize. For this, it is desired to provide a charge transport material having a high glass transition temperature (Tg) and excellent in thermal stability. Apart from charge transport materials heretofore known in the art, it is desired to further provide a material having a high charge transport efficiency.

Given the situation, the present inventors have made various investigations for the purpose of providing a novel compound which is stable in the amorphous state and hardly crystallizes and which has excellent characteristics as a charge transport material. In addition, the inventors have made further investigations for the purpose of providing an organic device such as an organic electroluminescence element, an organic thin-film solar cell and the like using an excellent charge transport material.

### Solution to Problem

The inventors have made assiduous studios for the purpose of solving the above-mentioned problems and, as a result, have found that a compound having multiple specific cyclic structures in the molecule thereof is thermally stable and has excellent characteristics as a charge transport material, and that the compound is effectively usable in organic devices. Based on these findings, the inventors have provided the present invention mentioned below as a solution to the problems.

(1) A compound represented by the following general formula [1]:

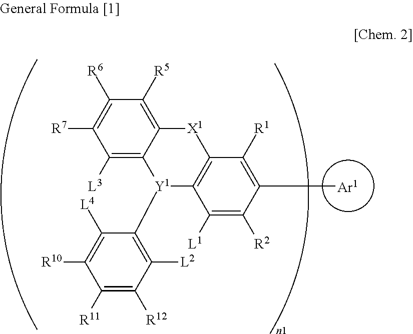

[In the general formula [1], Ar¹ represents a single bond or any of the following structures:

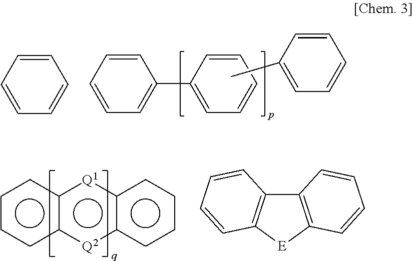

Q¹ and Q² are both ═CH—, or Q¹ is a single bond and Q² is —CH═CH—, or Q¹ is —CH═CH— and Q² is a single bond; p indicates an integer of from 0 to 3; q indicates an integer of from 0 to 3; E represents an oxygen atom or a sulfur atom, or represents an atomic group that links to the formula via a carbon atom, a silicon atom, a nitrogen atom, a phosphorus atom, a boron atom or a sulfur atom;
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom;
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom and a silicon atom; the other of L¹ and L², and L³ and L⁴ each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates an integer of 2 or more, and n1's X¹, Y¹, R¹, R², R⁵ to R⁷ and R¹⁰ to R¹² existing in the molecule may be the same or different;
when Ar¹ is a single bond, then the adjacent two R¹'s may bond to each other to form a linking group, and the adjacent two R²'s may bond to each other to form a linking group.]
(2) The compound according to (1), wherein in the general formula [1], the linking group formed by either one of L¹ and L², and L³ and L⁴, and the linking group represented by X¹ each are independently —O—, —S—, —SO₂—, >CR²¹R²², >C═O, >C═CR²³R²⁴, >C═NR²⁵, >NR²⁶,

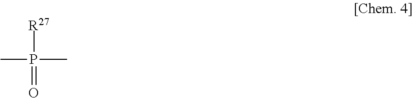

### or >SiR²⁸R²⁹;

### Y¹ is >N—, >B—, >P— or >P(═O)—;

R¹, R², R²¹, R²², R²⁸ and R²⁹ each are independently a hydrogen atom, a substituted or unsubstituted alkyl group, or a substituted or unsubstituted alkoxy group;
R⁵ to R⁷ and R¹⁰ to R¹² each are independently a hydrogen atom, a substituted or unsubstituted alkyl group, a substituted or unsubstituted alkoxy group, a substituted or unsubstituted aryl group, or a substituted or unsubstituted aryloxy group, or R⁵ and R⁶, R⁶ and R⁷, R¹⁰ to R¹² bond to each other to form a linking group; and
R²³ to R²⁷ each are independently a hydrogen atom, a substituted or unsubstituted alkyl group, or a substituted or unsubstituted aryl group.
(3) The compound according to (1) or (2), wherein in the general formula [1], the linking group formed by either one of L¹ and L², and L³ and L⁴, and the linking group represented by X¹ each are —O—.
(4) The compound according to any one of (1) to (3), wherein in the general formula [1], Y¹ is >N—.
(5) The compound according to any one of (1) to (4), wherein in the general formula [1], R¹ and R² each are a hydrogen atom.
(6) The compound according to any one of (1) to (5), wherein in the general formula [1], R⁵, R⁷, R¹⁰ and R¹² each are a hydrogen atom, and R⁶ and R¹¹ each are a hydrogen atom or an alkoxy group.
(7) The compound according to any one of (1) to (6), wherein the molecule is asymmetric.
(8) A charge transport material comprising the compound of any one of (1) to (7).
(9) An organic device using the compound of any one of (1) to (7).
(10) An electroluminescence element using the compound of any one of (1) to (7).
(11) A photoelectric conversion element using the compound of any one of (1) to (7).
(12) An organic thin-film solar cell using the compound of any one of (1) to (7).

### Advantageous Effects of Invention

The compound of the invention is stable in the amorphous state and hardly crystallizes and, in addition, has excellent characteristics as a charge transport material. Further, the organic device such as the organic electroluminescence element, the organic thin-film solar cell and the like of the present invention using the compound is highly efficient, and can retard the consumption power and the amount of heat generation and can realize long-life operation.

### BRIEF DESCRIPTION OF EMBODIMENTS

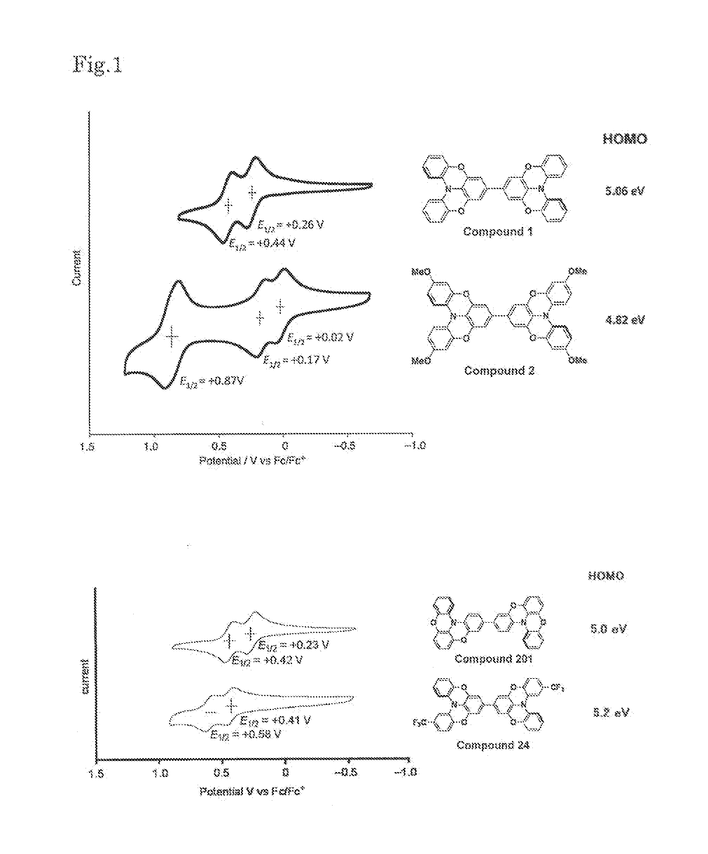

FIG. 1 This is a graph showing the measurements of cyclic voltammetry of compounds of the invention.

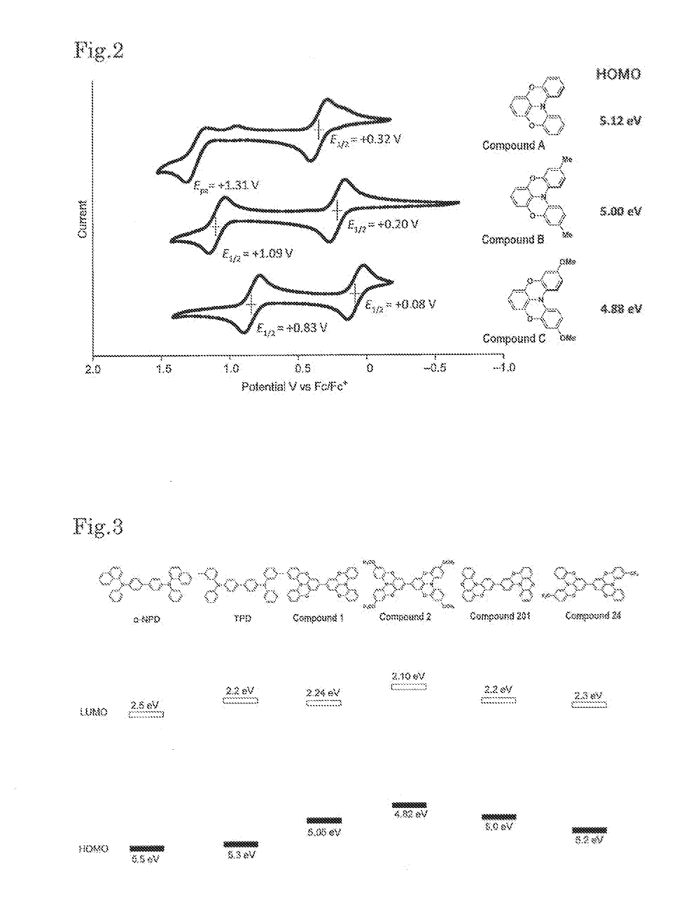

FIG. 2 This is a graph showing the measurements of cyclic voltammetry of comparative compounds A to C.

FIG. 3 This shows orbital levels of HOMO and LUMO.

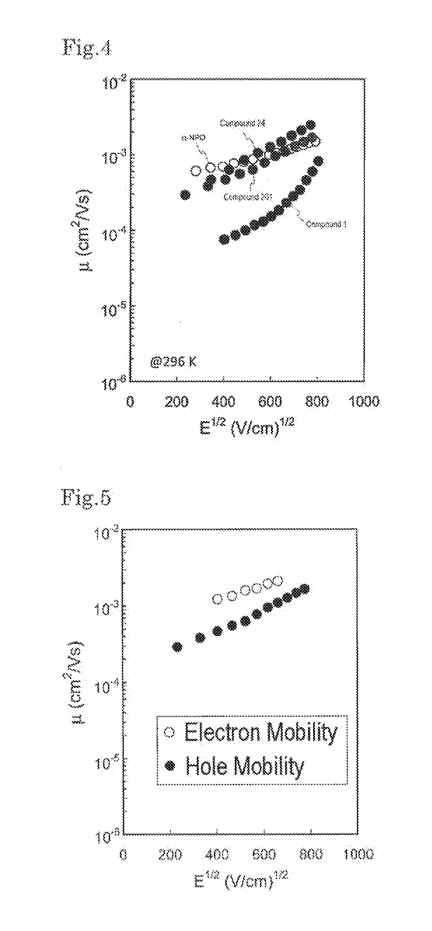

FIG. 4 This is a graph showing the data of hole mobility measured according to the TOF method in Example 9.

FIG. 5 This is a graph showing the data of compound 201 measured according to the TOF method in Example 9.

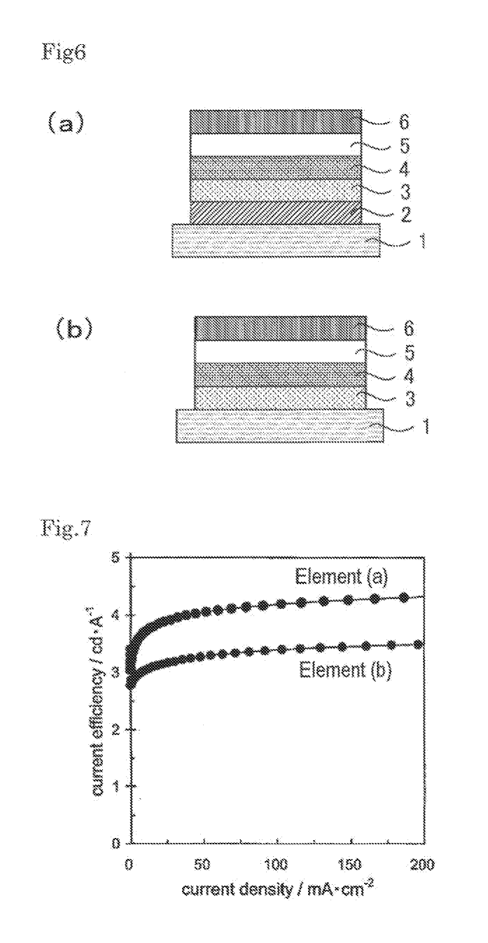

FIG. 6 This shows schematic cross-sectional views of the organic electroluminescence elements produced in Example 10.

FIG. 7 This in a graph showing the relationship between the current density and the current efficiency of the organic electroluminescence elements in Example 10.

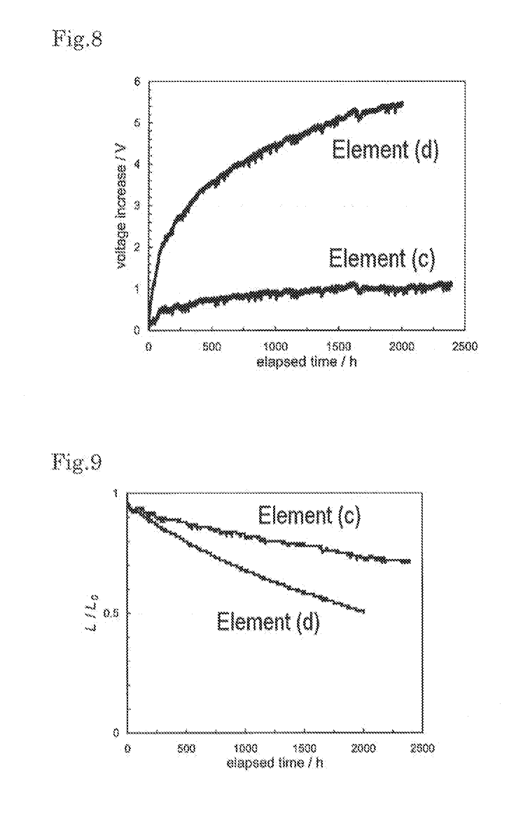

FIG. 8 This is a graph showing the relationship between the time and the voltage of the organic electroluminescence elements in Example 11.

FIG. 9 This is a graph showing the relationship between the time and the brightness of the organic electroluminescence elements in Example 11.

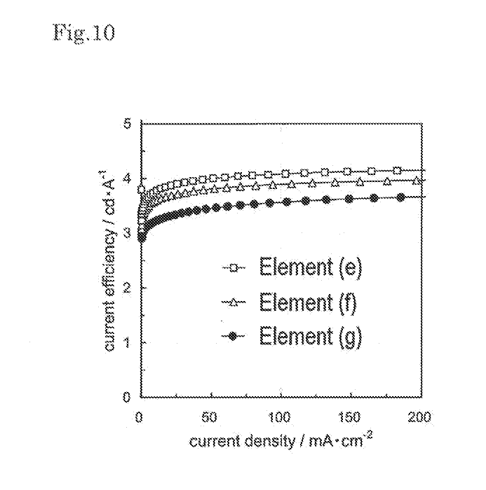

FIG. 10 This is a graph showing the relationship between the current density and the current efficiency of the organic electroluminescence elements in Example 12.

### DESCRIPTION OF EMBODIMENTS

The contents of the invention are described in detail hereinunder. The description of the constitutive elements of the invention given hereinunder is for some typical embodiments and specific examples of the invention; however, the invention should not be limited to such embodiments and specific examples. In this description, the numerical range expressed by the wording “a number to another number” means the range that falls between the former number indicating the lower limit of the range and the latter number indicating the upper limit thereof.

### [Compound Represented by General Formula [1]]

The compound of the invention has a structure represented by the following general formula [1]:

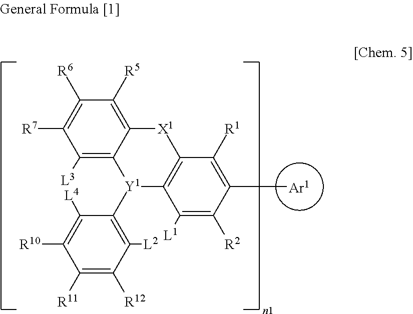

In the general formula [1], Ar¹ represents a single bond or a structure of any of the following [31] to [34];

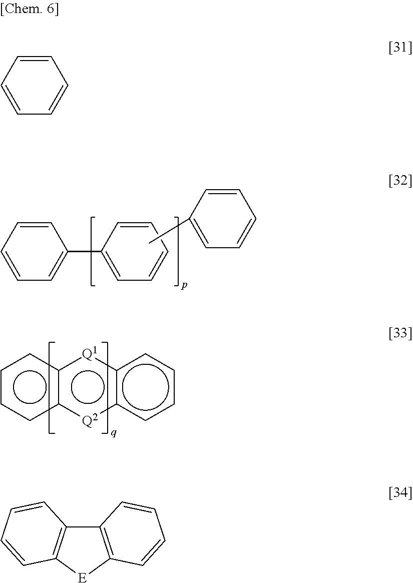

In case where Ar¹ is a benzene ring represented by the formula [31], the bonding position thereof when n1 is 2 includes 1,3-positions or 1,4-positions. The bonding position when n1 is 3 includes 1,3,5-positions.

When Ar¹ is represented by the general formula [32], p indicates an integer of from 0 to 3. For example, the bonding position of the biphenyl structure where p is 0 and n1 is 2 includes 3,3′-positions, or 4,4′-positions. When p is an integer of from 1 to 3, preferably, p's phenylene groups each are independently a 1,3-phenylene group or a 1,4-phenylene group. When p is 2 or 3, then the bonding positions of p's phenylene groups may be the same or different.

When Ar¹ is represented by the general formula [33], q indicates an integer of from 0 to 3. Q¹ and Q² are both ═CH—; or Q¹ is a single bond and Q² is —CH═CH—or Q¹ is —CH═CH— and Q² is a single bond. For example, the bonding position of the naphthalene structure where q is 0 and n1 is 2 includes 1,5-positions, 2,6-positions, 2,7-positions, or 1,8-positions. When q is 2 or 3, 1's Q¹'s may be the same or different, and q's Q²'s may be the same or different.

When Ar¹ is represented by the general formula [34], E represents an oxygen atom or a sulfur atom, or represents an atomic group that links to the formula via a carbon atom, a silicon atom, a nitrogen atom, a phosphorus atom, a boron atom or a sulfur atom, The general formula [34] includes the following general formulae [41], [42] and [43].

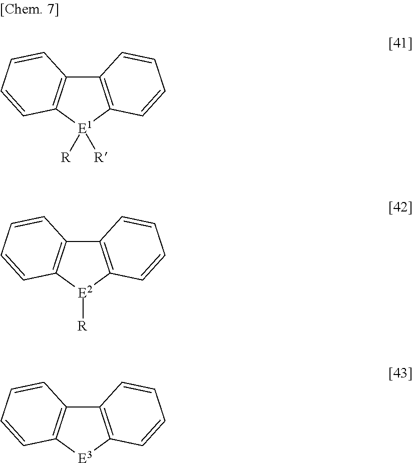

In the general formula [41], E¹ represents C or Si; in the general formula [42], E² represents N, P, P(═O) or B; in the general formula [43], E³ represents S, SO₂ or O. In the general formulae [41] and [42], R and R′ each independently represent a hydrogen atom or a substituent. For example, the substituent is preferably a substituted or unsubstituted alkyl group, or a substituted or unsubstituted aryl group. For preferred ranges of the substituents, referred to are the preferred ranges of the alkyl group and the aryl group directed to R²¹ to R²³ to be mentioned below.

In the general formula [1], X¹ represents a linking group that links to the formula via one atom selected from a group consisting of an oxygen atom, a sulfur atom, a carbon atom, a nitrogen atom, a phosphorus atom and a silicon, atom. Either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom and a silicon atom. The linking group represented by X¹ and the linking group represented by either one of L¹ and L², and L³ and L⁴ may be the same or different, but are preferably the same. The linking group that links via an oxygen atom, is —O—.

The linking group that links via a sulfur atom, is preferably —S— or —SO₂—, more preferably —S—.

The linking group that links via a carbon atom is preferably >CR²¹R²², >C═O, >C═CR²³R²⁴ or >C═NR²⁵, R²¹ to R²⁵ each independently represent a hydrogen atom or a constituent. Preferably, R²¹ and R²² each independently represent a hydrogen atom, a substituted or unsubstituted alkyl group, a substituted or unsubstituted alkoxy group, a substituted or unsubstituted aryl group, or a substituted or unsubstituted aryloxy group. Preferably, R²³ to R²⁵ each independently represent a hydrogen atom, a substituted or unsubstituted alkyl group, or a substituted or unsubstituted aryl group.

The linking group that links via a nitrogen atom is >NR²⁶. R²⁶ is preferably a hydrogen atom, a substituted or unsubstituted alkyl group, or a substituted or unsubstituted aryl group.

The linking group that links via a phosphorus atom is preferably the following:

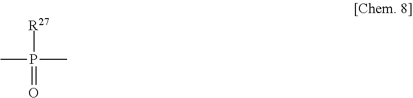

R²⁷ is preferably a hydrogen atom, a substituted or unsubstituted alkyl group, or a substituted or unsubstituted aryl group.

The linking group that links via a silicon atom is preferably >CR²⁸R²⁹. R²⁸ and R²⁹ each independently represent a hydrogen atom or a substituent. Preferably, R²⁶ and R²³ each are independently a hydrogen atom, a substituted or unsubstituted alkyl group, a substituted or unsubstituted alkoxy group, a substituted or unsubstituted aryl group, or a substituted or unsubstituted aryloxy group.

The alkyl group to be represented by R²¹ to R²⁹ may be linear, branched or cyclic. Preferably, the alkyl group is a linear or branched alkyl group. Preferably, the alkyl group has from 1 to 20 carbon atoms, more preferably from 1 to 12 carbon atoms, even more preferably from 1 to 6 carbon atoms, still more preferably from 1 to 3 carbon atoms (that is, a methyl group, an ethyl group, an n-propyl group, an isopropyl group). The cyclic alkyl group includes, for example, a cyclopentyl group, a cyclohexyl group, a cycloheptyl group.

The alkoxy group to be represented by R²¹, R²², R²⁸ and R²⁹ maybe linear, branched or cyclic. Preferably, the alkoxy group is a linear or branched alkoxy group. Preferably, the alkoxy group has from 1 to 20 carbon atoms, more preferably from 1 to 12 carbon atoms, even more preferably from 1 to 6 carbon atoms, still more preferably from 1 to 3 carbon atoms (that is, a methoxy group, an ethoxy group, an n-propoxy group, an isopropoxy group). The cyclic alkoxy group includes, for example, a cyclopentyloxy group, a cyclohexyloxy group, a cycloheptyloxy group.

The aryl group to be represented by R²¹ to R²⁹ may comprise one aromatic group or may have a fused structure of two or more aromatic rings. Preferably, the aryl group has from 6 to 22 carbon atoms, more preferably from 6 to 81 carbon atoms, even more preferably from 6 to 14 carbon atoms, still more preferably from 6 to 10 carbon atoms (that is, a phenyl group, a 1-naphthyl group, a 2-naphthyl group).

The aryloxy group no be represented by R²¹, R²², R²⁸ and R²⁹ may comprise one aromatic group or may have a fused structure of two or more aromatic rings. Preferably, the aryloxy group has from 6 to 22 carbon atoms, more preferably from 6 to 18 carbon atoms, even more preferably from 6 to 14 carbon atoms, still more preferably from 6 to 10 carbon atoms (that is, a phenyloxy group, a 1-naphthyloxy group, a 2-naphthyloxy group).

The alkyl group and the alkoxy group may be further substituted or may not be substituted. The substituent for the case where the group is substituted includes, for example, an alkoxy group, an aryl group and an aryloxy group; and for their descriptions and preferred ranges, referred to are those described for the above-mentioned alkoxy group, aryl group and aryloxy group.

The aryl group and the aryloxy group may be further substituted or may not be substituted. The substituent for the case where the group is substituted includes, for example, an alkyl group, an alkoxy group, an aryl group and an aryloxy group; and for their descriptions arid preferred ranges, referred to are those described for the above-mentioned alkyl group, alkoxy group, aryl group and aryloxy group.

In the general formula [1], Y¹ represents a linking group that links to the formula via one atom selected from a group consisting of a nitrogen atom, a boron atom and a phosphorus atom. The linking group chat links via a nitrogen atom is >N—. The linking group that links via a boron atom is >B—. The linking group that links via a phosphorus atom is preferably >P— or >P(═O)—.

When Y¹ is >N— or >P—, the compound of the general formula [1] exhibits properties useful as a charge transport material and exhibits properties useful especially as a hole transport material. When Y¹ is >B— or >P(═O)—, the compound of the general formula [1] exhibits properties useful as a charge transport material and exhibits properties useful especially as an electron transport material. Further, when Y¹ is >N—, the compound includes those exhibiting properties useful as a bipolar material, and especially when X¹ is —O—, the tendency is noted.

In the general formula [1], either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom and a silicon atom; the other of L¹ and L², and L³ and L⁴ each independently represent a hydrogen atom or a substituent. In other words, when L¹ and L² bond to each other to form the above-mentioned linking group, then L³ and L⁴ each are independently a hydrogen atom or a substituent; and when L³ and L⁴ bond to each other to form the above-mentioned linking group, then L¹ and L² each are independently a hydrogen atom or a substituent.

In the general formula [1], R¹, R², R⁵ to R⁷ and R¹⁰ to R¹² each independently represent a hydrogen atom or a substituent.

The substituent to be represented by R¹, R², R⁵ to R⁷, or R¹⁰ to R²², and L¹ to L⁴ includes, for example, a substituted or unsubstituted alkyl group, a substituted or unsubstituted alkoxy group, a substituted or unsubstituted aryl group, and a substituted or unsubstituted aryloxy group. For the descriptions and the preferred ranges of there substituents, referred to are the descriptions of the above-mentioned alkyl group, alkoxy group, aryl group and aryloxy group.

In the general formula [1], preferably, R¹ and R² each are independently a hydrogen atom, a substituted or unsubstituted alkyl group, or a substituted or unsubstituted alkoxy group. Also preferably, when Ar¹ is a single bond, then the adjacent two R¹'s bond to each ether to form a linking group, or the adjacent two R²'s bond to each other to form a linking group. For the descriptions and the preferred ranges of the alkyl group and the alkoxy group, referred to are the descriptions of the above-mentioned alkyl group and alkoxy group. More preferably, R¹ and R² each are a hydrogen atom, a methyl group or a methoxy group. Also preferably, R¹ and R² are both hydrogen atoms.

When the adjacent two R¹'s bond to each other to form a linking group, preferably, the linking group links via one atom selected from a group consisting of an oxygen atom, a sulfur atom, a carbon atom, a nitrogen atom or a phosphorus atom. Concretely, the linking group is represented by —O—, —S—, —SO₂—, >CR²¹R²², >C═O, >C═CR²³R²⁴, >C═NR²⁵, >NR²⁶ or

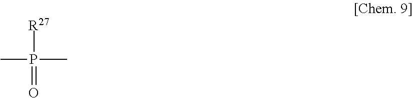

or >SiR²⁸R²⁹. For the descriptions are the preferred ranges of these linking groups, referred to are the descriptions of the corresponding linking groups of the above-mentioned X¹ and X². The descriptions and the preferred ranges of the case where the adjacent two R²'s bond to each other to form a linking group are the same as those of the case where the adjacent two R¹'s bond to each other to form a linking group. Both the adjacent two R¹'s and the adjacent two R²'s may bond to each other to form a linking group; but either one of these may bond to each other to form a linking group.

In the general, formula (1) preferably, R⁵ to R⁷ and R¹⁰ to R¹² each are independently a hydrogen atom, a substituted or unsubstituted alkyl group, a substituted or unsubstituted alkoxy group, a substituted or unsubstituted aryl group, or a substituted or unsubstituted aryloxy group. For the descriptions and the preferred ranges of these substituents, referred to are the descriptions of the above-mentioned alkyl group, alkoxy group, aryl group and aryloxy group.

More preferably, L¹ to L⁴ not forming a linking group each are a hydrogen atom, an alkyl group having from 1 to 3 carbon atoms, or an alkoxy group having from 1 to 3 carbon, atoms, even more preferably a hydrogen atom, a methyl group or a methoxy group. Preferably, all L¹ to L⁴ not forming a linking group are hydrogen atoms.

All of R⁵ to R⁷ and R¹⁰ to R¹² may be hydrogen atoms, or at least one of them may be a substituent. In the case where at least one is a substituent, more preferably, at least one of R⁶, R⁷, R¹⁰ and R¹¹ is a substituent.

In the general formula [1], R⁵ and R⁶, R⁶ and R⁷, R¹⁰ and R¹¹, and R¹¹ and R¹² each may bond to each other to form a linking group. The linking group to be formed is preferably one in which the linking chain comprises at least one atom selected from a group consisting of a carbon atom, an oxygen atom, a sulfur atom, a nitrogen atom and a phosphorus atom. For example, a linking chain composed of carbon atoms alone is mentioned as one preferred example. The linking chain composed of carbon atoms alone may contain a double bond, or may comprise a single bond alone. Preferably, the carbon number of the linking chain is from 2 to 6, more preferably from 3 to 5, even more preferably 3 or 4, and most preferably 4. A hydrogen atom or a substituent may bond to the atoms constituting the linking chain. One preferred example of the linking group has a structure represented by:

—C(R³⁰)═C(R³¹)—C(R³²)═C(R³³)—,

wherein R³⁰ to R³³ each represent a hydrogen atom or a substituent; R³⁰ and R³¹, and R³¹ and R³², and R³² R³³ each may bond to each other to further form a linking group. The substituent as referred to herein includes, for example, an alkyl group, an alkoxy group, an aryl group and an aryloxy group; and for their descriptions and preferred ranges, referred to are the descriptions of the above-mentioned alkyl group, alkoxy group, aryl group and aryloxy group. For the descriptions and the preferred ranges of the linking group to be formed by R³⁰ and R³¹ and the like, referred to are the descriptions of the linking group to be formed by the above-mentioned R⁵ and R⁶, etc.

In the general formula [1], n1 is an integer of 2 or more. n1 is preferably an integer of from 2 to 10, more preferably an integer of from 2 to 4. For example, n1 may be 2 or 3.

Preferred ranges of the compound represented by the general formula [1] are mentioned, in which either one of L¹ and L², and L³ and L⁴, and X¹ each are independently a linking group selected from —O—, —S—, —SO₂—, >CR²¹R²², >C═O, >C═CR²³R²⁴, >C═NR²⁵, >NR²⁶,

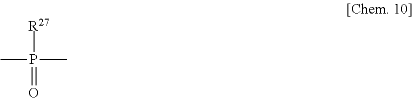

or >SiR²⁸R²⁹; Y¹ is >N—, >B—, >P— or >P(═O)—; R¹R², R²¹, R²², R²⁸ and R²⁹ each are independently a hydrogen atom, a substituted or unsubstituted alkyl group, or a substituted or unsubstituted alkoxy group, or when Ar¹ is a single bond, the adjacent two R¹'s bond to each other to form a linking group, or the adjacent two R²'s bond to each other to form a linking group; L¹ to L⁴ not forming a linking group, R⁵ to R⁷ and R¹⁰ to R¹² each are independently a hydrogen atom, a substituted or unsubstituted alkyl group, a substituted or unsubstituted alkoxy group, a substituted or unsubstituted aryl group, or a substituted or unsubstituted aryloxy group, or R⁵ and R⁶, R⁶ and R⁷, R¹⁰ and R¹¹, or R¹¹ and R¹² each bond to each other to form a linking group; R²³ to R²⁷ each are independently a hydrogen atom, a substituted or unsubstituted alkyl group, or a substituted or unsubstituted aryl group; and n1 is as integer of from 2 to 6.

Preferred structures of the general formula [1] are the following general formula [1-1] and general formula [1-2]. For the definitions and the preferred ranges of Ar¹, X¹, Y¹, R¹, R², R⁵ to R⁷, R¹⁰ to R¹² and n1 in the general formulae [1-1] and [1-2], referred to are the corresponding descriptions of the general formula [1]. The definitions and the preferred ranges of X² and X³ are the same as the definitions and the preferred ranges of X¹ in the general formula [1]. X¹ to X³ may be the same or different. L¹¹ to L¹⁴ each are independently a hydrogen atom or a substituent. For the definitions and the preferred ranges of the substituents for L¹¹ to L¹⁴, referred to are the descriptions of the substituents for L¹ to L⁴ not forming a linking group in the general formula [1]:

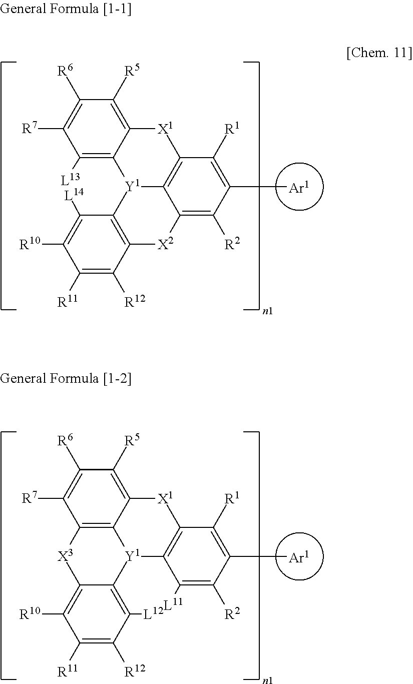

As other preferred ranges of the compound represented by the general formula [1], there may be mentioned the compounds represented by the following general formula [2]:

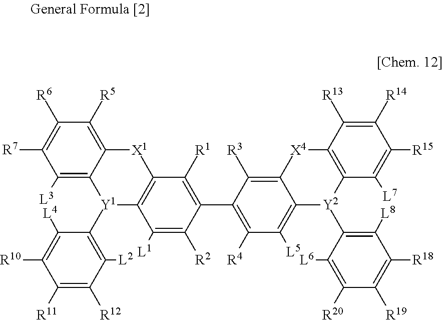

In the general formula [2], X¹ and X⁴ each independently represent a linking group that links to the formula via one atom selected from a group consisting of an oxygen atom, a sulfur atom, a carbon atom, a nitrogen atom, a phosphorus atom and a silicon atom. For the descriptions and the preferred ranges of X¹ and X⁴, referred to are the descriptions of X¹ in the general formula [1] given hereinabove. X¹ and X⁴ may be the same or different, but are preferably the same.

Either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom and a silicon atom. Either one of L⁵ and L⁶, and L⁷ and L⁸ bond to each other to represent a linking group that links to the formula via one atom selected from a group consisting of an oxygen atom, a sulfur atom, a carbon atom, a nitrogen atom, a phosphorus atom and a silicon atom. When L¹ and L² bond to each other to form a linking group, then preferably, L⁵ and L⁶ bond to each other to form a linking group. When L³ and L⁴ bond to each other to form a linking group, then preferably, L⁷ and L⁸ bond to each ether to form a linking group. The linking group to be formed by either one of L¹ and L², and L³ and L⁴ bonding to each other, and the linking group to be formed by either one of L⁵ and L⁶, and L⁷ and L⁸ bonding to each other may be the same or different, but are preferably the same. The linking group to be formed by either one of L¹ and L², and L³ and L⁴ bonding no each other, the linking group to be formed by either one of L⁵ and L⁶, and L⁷ and L⁸ bonding to each other, and the linking group to be formed by X¹ and X⁴ may be the same or different, but are preferably the same.

Y¹ and Y² each independently represent a linking group that links to the formula via one atom selected from a group consisting of a nitrogen atom, a boron atom and a phosphorus atom. For the description, and the preferred ranges of Y¹ and Y², referred to are the descriptions of Y¹ in the general formula [1] given hereinbefore. Y¹ and Y² may be the same or different, but are preferably the same.

In the general formula [2], either one of L1 and L², and L³ and L⁴ bond to each other to form a linking group that links to the formula via one atom selected from a group consisting of an oxygen atom, a sulfur atom, a carbon atom, a nitrogen atom, a phosphorus atom and a silicon atom, and the other of L¹ and L², and L³ and L⁴ each independently represent a hydrogen atom or a substituent. In other words, when L¹ and L² bonds to each other to form the above-mentioned linking group, then L³ and L⁴ each independently represent a hydrogen atom or a substituent; and when L³ and L⁴ bonds to each other to form the above-mentioned linking group, then L¹ and L² each independently represent a hydrogen atom, or a substituent.

Similarly, either one of L⁵ and L⁶, and L⁷ and L⁸ bond to each other to form a linking group that links to the formula via one atom selected from a group consisting of an oxygen atom, a sulfur atom, a carbon atom, a nitrogen atom, a phosphorus atom and a silicon atom, and the other of L⁵ and L⁶, and L⁷ and L⁸ each independently represent a hydrogen atom or a substituent. In other words, when L⁵ and L⁶ bonds to each other to form the above-mentioned linking group, then L⁷ and L⁸ each independently represent a hydrogen atom or a substituent; and when L⁷ and L⁸ bonds to each other to form the above-mentioned, linking group, then L⁵ and L⁶ each independently represent a hydrogen atom or a substituent.

L¹ to L⁸, R¹ to R⁴, R⁵ to R⁷, R¹⁰ to R¹², R¹³ to R¹⁵, and R¹⁸ to R²⁰ not forming a linking group are each independently a hydrogen atom or a substituent; and R¹ and R³, R² and R⁴, R⁵ and R⁶, R⁶ and R⁷, R¹⁰ and R¹¹, R¹¹ and R¹², R¹³ and R¹⁴, R¹⁴ and R¹⁵, R¹⁸ and R¹⁹, and R¹⁹ and R²⁰ each may bond to each other to form a linking group. For the descriptions and the preferred ranges of R¹ to R⁴, referred to are the descriptions of R¹ and R² in the general formula [1] given hereinabove. For the descriptions and the preferred ranges of R⁵ to R⁷, R¹⁰ to R¹², R¹³ to R¹⁵ and R¹⁸ to R²⁰, referred to are the descriptions of R⁵ and R¹² in the general formula [1] given hereinabove.

More preferably, L¹ to L⁶ not forming a linking group each are independently a hydrogen atom, an alkyl group having from 1 to 3 carbon atoms, or an alkoxy group having from 1 to 3 carbon atoms, and even more preferably a hydrogen atom, a methyl group or a methoxy group. Preferably, L¹ to L⁸ not forming a linking group are all hydrogen atoms.

All of R¹ to R⁴, R⁵ to R⁷, R¹⁰ to R¹², R¹³ to R¹⁵, and R¹⁸ to R²⁰ may be hydrogen atoms, or at least one of them may be a substituent. In case where at least one is a substituent, preferably, at least one of R⁵ to R⁷, R¹⁰ to R¹², R¹³ to R¹⁵ and R¹⁸ to R²⁰ is a substituent, and more preferably at least one of R⁶, R¹¹, R¹⁴ and R¹⁹ is a substituent. In case where at least one of R⁶, R¹¹, R¹⁴ and R¹⁹ is a substituent, preferably, at least two of R⁶, R¹¹, R¹⁴ and R¹⁹ are substituents, and more preferably, ail of them are substituents.

As preferred structures of the general formula [2], there are mentioned the following general formula [2-1] and general formula [2-2]. For the definitions and the preferred ranges of X¹, X⁴, Y¹, Y², R¹ to R⁴, R⁵ to R⁷, R¹⁰ to R¹², R¹³ to R¹⁵ and R¹⁸ to R²⁰ in the general formulae [2-1] and [2-2], referred to are the corresponding descriptions of the general formula [2]. The definitions and the preferred ranges of X², X³, X⁵ and X⁶ are the same as the definitions and the preferred ranges of X¹ in the general formula [2]. X¹ to X⁶ may be the same or different. L¹¹ to L¹⁸ each independently represent a hydrogen atom or a substituent. For the definitions and the preferred ranges of L¹¹ to L¹⁸, referred to are the descriptions of the substituents for L¹ to L⁴ not forming a linking group in the general formula [1].

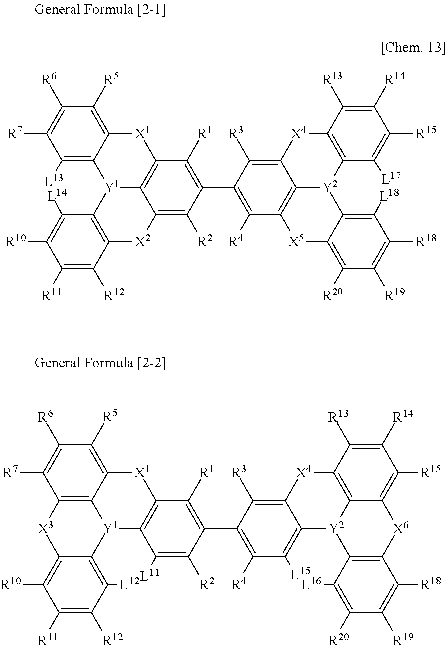

As other preferred ranges of the compound represented by the general formula [1], there may be mentioned the compounds represented by the following general formula [3]:

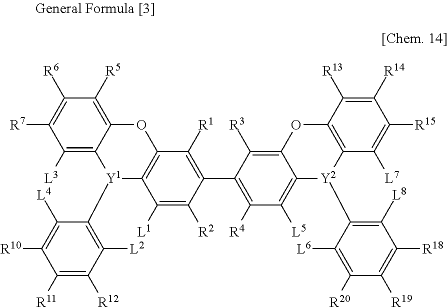

For the definitions and the preferred ranges of Y¹, Y², R¹ to R⁴, R⁵ to R⁷, R¹⁰ to R¹², R¹³ to R¹⁵ and R¹⁸ to R²⁰ in the general formula [3], referred to are the corresponding descriptions of the general formulae [1] and [2].

Either one of L¹ and L², and L³ and L⁴ in the general formula [3] bond to each order to form a linking group (—O—) that links via an oxygen atom. Either one of L⁵ and L⁶, and ⁷ and L⁸ bond to each other to form a linking group (—O—) that links via an oxygen atom. In case where L¹ and L² bond to each other to form a linking group, preferably, L⁵ and L⁶ bond to each other to form a linking group. In case where L³ and L⁴ bond to each other to form a linking group, preferably, L⁷ and L⁸ bond to each other to form a linking group.

In the general formula [3], either one of L¹ and L², and L³ and L⁴ bond to each other to form a linking group (—O—), and the other of L¹ and L², and L³ and L⁴ each are independently a hydrogen atom or a substituent. In other words, when L¹ and L² bond to each other to form a linking group (—O—), then L³ and L⁴ each are independently a hydrogen atom or a substituent; and when L³ and L⁴ bond to each ether to form a linking group (—O—), then L¹ and L² each are independently a hydrogen atom or a substituent.

Similarly, either one of L⁵ and L⁶, and L⁷ and L⁸ bond to each other to form a linking group (—O—), and the other of L⁵ and L⁶, and L⁷ and L⁸ each are independently a hydrogen atom, or a substituent. In other words, when L⁵ and L⁶ bond to each other to form a linking group (—O—) , then L⁷ and L⁸ each are independently a hydrogen atom or a substituent; and when L⁷ and L⁸ bond to each other to form a linking group (—O—), then L⁵ and L⁶ each are independently a hydrogen atom or a substituent.

One preferred range of the compound represented by the general formula [3] is mentioned, in which R¹ to R⁴ each are a hydrogen atom, an alkyl group having from 1 to 3 carbon atoms, or an alkoxy group having from 1 to 3 carbon atoms. In the range, more preferably, R¹ to R⁴ each are a hydrogen atom, a methyl group or a methoxy group, and even more preferably, R¹ to R⁴ are all hydrogen atoms.

Another preferred range of the compound represented by the general formula [3] is mentioned, in which R¹ and R³ bond to each other to form a linking group. More preferably, R¹ and R³ bond to each other to form a linking group that links to the formula via one atom selected from a group consisting of an oxygen atom, a sulfur atom, a carbon atom, a nitrogen atom end a phosphorus atom, and even more preferably, R¹ and R³ bond to each other to form a linking group represented by —O—, —S—, —SO₂—, >CR²¹R²², >C═O, >C═CR²³ R²⁴, >C═NR²⁵, >NR²⁶ or

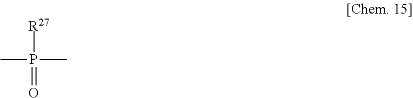

or >SiR²⁸R²⁹. Preferably, R² and R⁴ are both hydrogen atoms, or bond to each other to form a linking group. The descriptions and the preferred ranges of the linking group to be formed by R² and R⁴ are the same as those of the linking group to be formed by R¹ and R³ in the general formula [3]. As one concrete example, there is mentioned a case where R¹ and R³ bond to each other to form —O— and R² and R⁴ are both hydrogen atoms, As another concrete example, there is mentioned a case where R¹ and R² bond to each other to form —O— and R² and R⁴ also bond to each other to form —O—.

As preferred structures of the general formula [3], there are mentioned the following general formula [3-1] and general formula [3-2]. For the definitions and the preferred ranges of Y¹, Y², R¹ to R⁴, R⁵ to R⁷, R¹⁰ to R¹², R¹³ to R¹⁵ and R¹⁸ to R²⁰ in the general formulae [3-2] and [3-2], referred to are the corresponding descriptions of the general formula [3].

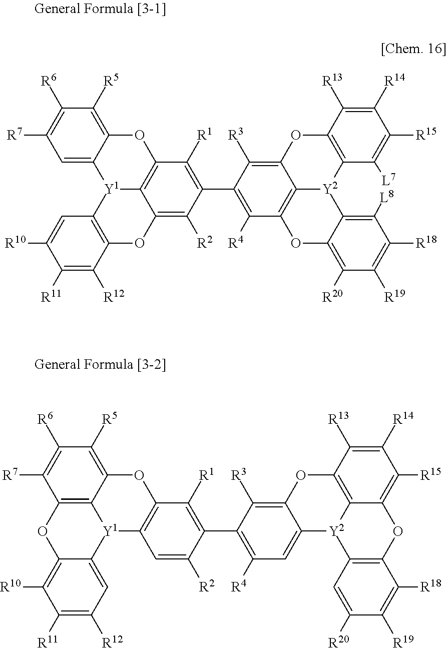

As other preferred ranges of the compound represented by the general formula [1], there may be mentioned the compounds represented by the following general formula [4-1], general formula [4-2], general formula [4-1] and general formula [4-4]:

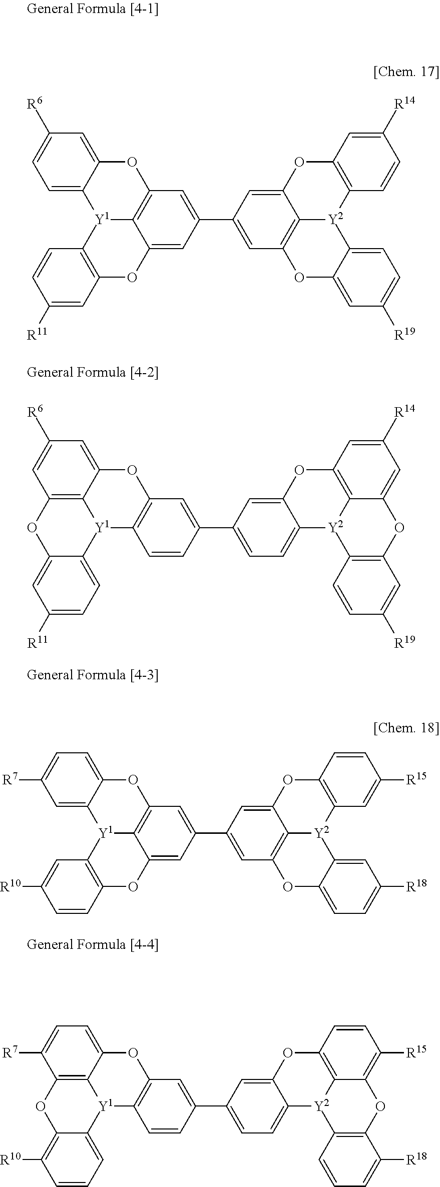

For the definitions and the preferred ranges of Y¹, Y², R⁶, R⁷, R¹⁰, R¹¹, R¹⁴, R¹⁵, R¹⁸ and R¹⁹ in the general formula [4], referred to are the corresponding descriptions of the general formulae [1] and [2].

One preferred range of the compound represented by the general formula [4] is mentioned, in which Y¹ and Y² are both nitrogen atoms.

Another preferred range of the compound represented by the general formula [4] is mentioned, in which R⁶, R¹¹, R¹⁴ and R¹⁹ each are a hydrogen atom or a substituent. More preferably, R⁶, R¹¹, R¹⁴ and R¹⁹ are all hydrogen atoms, or any two of them are substituents, or all of them are substituents. Also mentioned is a case where R⁷, R¹⁰, R¹⁵ and R¹⁶ each are a hydrogen atom or a substituent. More preferably, R⁷, R¹⁰, R¹⁵ and R¹⁸ are all hydrogen atoms, or any two of them are substituents, or all of them are substituents. The substituent for R⁶, R⁷, R¹⁰, R¹¹, R¹⁴, R¹⁵, R¹⁸ and R¹⁹ is preferably a substituted, or unsubstituted alkyl group, a substituted or unsubstituted alkoxy group, a substituted or unsubstituted aryl group, or a substituted or unsubstituted aryloxy group. For the descriptions and the preferred ranges of the substituents, referred to are the description of the corresponding substituents in the general formula [1]. As specific examples, there are mentioned a case where R¹¹ and R¹⁴ are hydrogen atoms and R⁶ and R¹⁹ are alkoxy groups, and a case where R⁷ and R¹⁸ are trifluoromethyl groups and R¹⁰ and R¹⁵ are hydrogen atoms.

As other preferred ranges of the compound represented by the general formula [1], there may be mentioned the compounds represented by the following general formula [5]:

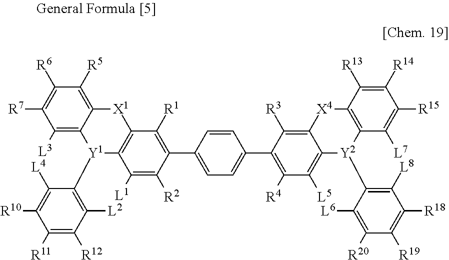

For the definitions and the preferred ranges of X¹, X⁴, Y¹, Y², R⁵ to R⁷, R¹⁰ to R¹², R¹³ to R¹⁵, R¹⁸ to R²⁰ in the general formula [5], referred to are the corresponding descriptions in the general formulae [1] to [3]. For the definitions and the preferred ranges of L¹ to L⁸ in the general formula [5], referred to are the corresponding descriptions in the general formula [2]. In the general formula [5], R¹ to R⁴ each independently represent a hydrogen atom or a substituent. For the descriptions and the preferred ranges or the substituent, referred to are the descriptions and the preferred ranges of the substituent for R¹ and R² in the general formula [1].

One preferred range of the compound represented by the general formula [5] is mentioned, in which, for example, X¹ and X⁴ are oxygen atoms; L¹ and L², and L⁵ and L⁶ each bond to each other to form a linking group (—O—) that links via an oxygen atom; Y¹ and Y² are nitrogen atoms; L³, L⁴, L⁷, L⁸, R¹ to R⁵, R⁷ to R¹⁰, R¹², R¹³, R¹⁵ and R²⁰ are hydrogen atoms; and R⁶, R¹¹, R¹⁴ and R¹⁹ each are independently a hydrogen atom, a substituted or unsubstituted alkyl group, a substituted or unsubstituted alkoxy group, a substituted or unsubstituted aryl group, or a substituted or unsubstituted aryloxy group.

Another preferred range of the compound represented by the general formula [5] is mentioned, in which, for example, X¹ and X⁴ are oxygen atoms; L³ and L⁴, and L⁷ and L⁸ each bond to each other to form a linking group (—O—) that links via an oxygen atom; Y¹ and Y² are nitrogen atoms; L¹, L², L⁵, L⁶, R¹ to R⁵, R⁷ to R¹⁰, R¹², R¹³, R¹⁵ to R¹⁸ and R²⁰ are hydrogen atoms; and R⁶, R¹¹, R¹⁴ and R¹⁹ each are independently a hydrogen atom, a substituted or unsubstituted alkyl group, a substituted or unsubstituted alkoxy group, a substituted or unsubstituted aryl group, or a substituted or unsubstituted aryloxy group.

As other preferred ranges of the compound represented by the general formula [1], there may be mentioned the compounds represented by the following general formula [6]:

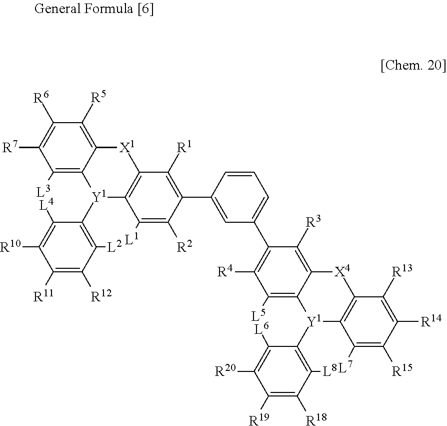

For the definitions and the preferred ranges of X¹, X⁴, Y¹, Y², L¹ to L⁸, R¹ to R⁴, R⁵ to R⁷, R¹⁰ to R¹², R¹³ to R¹⁵ and R¹⁸ to R²⁰ in the general formula [6], referred to are the corresponding descriptions in the general formula [5].

One preferred range of the compound represented by the general formula [6] is mentioned, in which, for example, X¹ and X⁴ are oxygen atoms; L¹ and L², and L⁵ and L⁶ each bond to each other to form a linking group (—O—) that links via an oxygen atom; Y¹ and Y² are nitrogen atoms; L³, L⁴, L⁷, L⁸, R¹ to R⁵, R⁷ to R¹⁰, R¹², R¹³, R¹⁵ to R¹⁸ and R²⁰ are hydrogen atoms; and R⁶, R¹¹, R¹⁴ and R¹⁹ each are independently a hydrogen atom, a substituted or unsubstituted alkyl group, a substituted or unsubstituted alkoxy group, a substituted or unsubstituted aryl group, or a substituted or unsubstituted aryloxy group.

Another preferred range of the compound represented by the general formula [6] is mentioned, in which, for example, X¹ and X⁴ are oxygen atoms; L³ and L⁴, and L⁷ and L⁸ each bond to each other to form a linking group (—O—) that links via an oxygen atom; Y¹ and Y² are nitrogen atoms; L¹, L², L⁵, L⁶, R¹ to R⁵, R⁷ to R¹⁰, R¹², R¹³, R¹⁵ to R¹⁸ and R²⁰ are hydrogen atoms; and R⁶, R¹¹, R¹⁴ and R¹⁹ each are independently a hydrogen atom, a substituted or unsubstituted alkyl group, a substituted, or unsubstituted alkoxy group, a substituted or unsubstituted aryl group, or a substituted or unsubstituted aryloxy group.

As other preferred ranges of the compound represented by the general formula [1], there may be mentioned the compounds represented by the following general formula [7]:

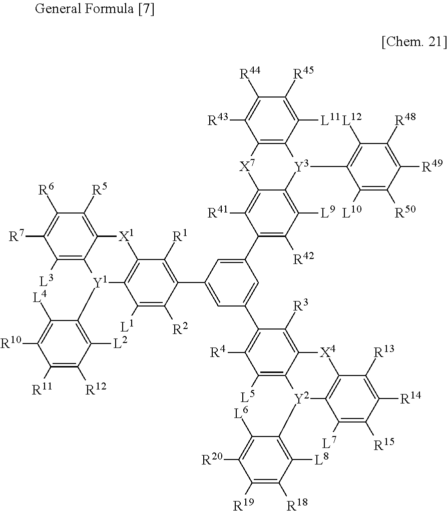

For the definitions and the preferred ranges of X¹, X⁴ and X⁷ in the general formula [7], referred to are the descriptions of X¹ in the general formula [1]. For the definitions and the preferred ranges of Y¹ to Y³ in the general formula [7], referred to are the descriptions of Y¹ in the general formula [1], for the definitions and the preferred ranges of R⁵ to R⁷, R¹⁰ to R¹², R¹³ to R¹⁵, R¹⁸ to R²⁰, R⁴³ to R⁴⁵ and R⁴⁸ to R⁵⁰ in the general formula [7], referred to are the descriptions of R⁵ to R⁷ and R¹⁰ to R¹² in the general formula [1]. For the definitions and the preferred ranges of R¹ to R⁴, R⁴¹ and R⁴² in the general formula [7], referred to are the descriptions of R¹ and R² in the general formula [1]. For the definitions and the preferred ranges of L¹ to L⁸ in the general formula [7], referred to are the corresponding descriptions in the general formula [2].

Either one of L⁹ and L¹⁰, and L¹¹ and L¹² in the general formula [7] bond to each other to form a linking group that links to the formula via one atom selected from a group consisting of an oxygen atom, a sulfur atom, a carbon atom, a nitrogen atom, a phosphorus atom and a silicon atom. The other of L⁹ and L¹⁰, and L¹¹ and L¹² each independently represent a hydrogen atom or a substituent. In other words, when L⁹ and L¹⁰ bond to each other to form the above-mentioned linking group, then L¹¹ and L¹² each are independently a hydrogen atom, or a substituent; and when L¹¹ and L¹² bond to each other to form the above-mentioned linking group, then L⁹ and L¹⁰ each are independently a hydrogen atom or a substituent. When L¹ and L² bond to each other to form a linking group, preferably, L⁵ and L⁶ bond to each other to form a linking group and L⁹ and L¹⁰ bond to each other to form a linking group. Also preferably, when L³ and L⁴ bond to each other to form a linking group, then L⁷ and L⁸ bond to each other to form a linking group and L¹¹ and L¹² bond to each other to form a linking group. The linking group to be formed by either one of L¹ and L², and L³ and L⁴, the linking group to be formed by either one of L⁵ and L⁶, and L⁷ and L⁸, and the linking group to be formed by either one of L⁹ and L¹⁰, and L¹¹ and L¹² may be the same or different, but are preferably the same. The linking group to be formed by either one of L¹ and L², and L³ and L⁴, the linking group to be formed by either one of L⁵ and L⁶, and L⁷ and L⁸, the linking group to be formed by either one of L⁹ and L¹⁰, and L¹¹ and L¹², and the linking group to be represented by X¹, X⁴ and X⁷ may be the same or different, but are preferably the same.

One preferred range of the compound represented by the general formula [7] is mentioned, in which, for example, X¹, X⁴ and X⁷ are oxygen atoms; Y¹ to Y³ are nitrogen atoms; R¹ to R⁵, R⁷, R¹⁰, R¹², R¹³, R¹⁵, R¹⁸, R²⁰, R⁴¹ to R⁴³, R⁴⁵, R⁴⁸ and R⁵⁰ are hydrogen atoms; and R⁶, R¹¹, R¹⁴, R¹⁹, R⁴⁴ and R⁴⁵ each are independently a hydrogen atom, a substituted or unsubstituted alkyl group, a substituted or unsubstituted alkoxy group, a substituted or unsubstituted aryl group, or a substituted or unsubstituted aryloxy group.

Another preferred range of the compound represented by the general formula [7] is mentioned, in which, for example, X¹, X⁴ and X⁷ are oxygen atoms; Y¹ to Y³ are nitrogen atoms; R¹ to R⁶, R¹¹ to R¹⁴, R¹⁹, R²⁰, R⁴¹ to R⁴³, R⁴⁴, R⁴⁹ and R⁵⁰ are hydrogen atoms; and R⁷, R¹⁰, R¹⁵, R¹⁸, R⁴⁵ and R⁴⁸ each are independently a hydrogen atom, a substituted or unsubstituted alkyl group, a substituted or unsubstituted alkoxy group, a substituted or unsubstituted aryl group, or a substituted or unsubstituted aryloxy group.

The molecules of the compounds represented by the general formulae [1] to [7] may have a symmetric structure or an asymmetric structure. “Symmetric” as referred to herein means line-symmetric or point-symmetric.

Specific examples of the compounds represented by the general formula [1] are shown below. The range of the compounds represented by the general formula [1] in the invention should not be limitatively interpreted by the following specific examples. The following Tables 1 and 2 show specific examples of the compounds represented by the general formula [2-1]; the following Tables 3 and 4 show specific examples of the compounds represented by the general formula [2-2]; the following Tables 5 and 6 show specific examples of the compounds represented by the general formula [5]; the following Tables 7 and 8 show specific examples of the compounds represented by the general formula [6]; and the following Tables 9 and 10 show specific examples of the compounds represented by the general formula [7].

TABLE 1

| Compound | General Formula [2-1] | | | | | | | | | | |
|---|---|---|---|---|---|---|---|---|---|---|---|
| No. | X¹, X², X⁴, X⁵ | Y¹, Y² | R¹, R³ | R², R⁴ | R⁵, R²⁹ | R¹², R¹³ | R⁵, R¹⁹ | R¹¹, R¹⁴ | R⁷, R³⁸ | R¹⁰, R³⁵ | L¹³, L¹⁴, L¹⁷, L¹⁸ |
| Compound 1 | —O— | >N— | H | H | H | H | H | H | H | H | H |
| Compound 2 | —O— | >N— | H | H | H | H | CH₃O | CH₃O | H | H | H |
| Compound 3 | —O— | >N— | H | H | H | H | CH₃O | H | H | H | H |
| Compound 4 | —O— | >N— | H | H | H | H | CH₃ | H | H | H | H |
| Compound 5 | —O— | >N— | H | H | H | H | C₆H₅O | C₆H₅O | H | H | H |
| Compound 6 | —O— | >N— | H | H | H | H | C₆H₅ | C₆H₅ | H | H | H |
| Compound 7 | —O— | >N— | H | H | H | H | CF₃O | CF₃ | H | H | H |
| Compound 8 | —O— | >N— | H | H | H | H | CF₃O | H | H | H | H |
| Compound 9 | —O— | >N— | H | H | H | H | H | H | H | H | H |
| Compound 10 | —O— | >N— | H | H | CH₃O | CH₃O | H | H | H | H | H |
| Compound 11 | —O— | >N— | H | H | CH₃O | H | H | H | H | H | H |
| Compound 12 | —O— | >N— | H | H | CH₃ | H | H | H | H | H | H |
| Compound 13 | —O— | >N— | H | H | C₆H₅O | C₆H₅O | H | H | H | H | H |
| Compound 14 | —O— | >N— | H | H | C₆H₅ | C₆H₅ | H | H | H | H | H |
| Compound 15 | —O— | >N— | H | H | CF₃O | CF₃O | H | H | H | H | H |
| Compound 16 | —O— | >N— | H | H | CF₃O | H | H | H | H | H | H |
| Compound 17 | —O— | >N— | H | H | H | H | H | H | H | H | H |
| Compound 18 | —O— | >N— | H | H | H | H | H | H | CH₃O | CH₃O | H |
| Compound 19 | —O— | >N— | H | H | H | H | H | H | CH₃O | H | H |
| Compound 20 | —O— | >N— | H | H | H | H | H | H | CH₃ | H | H |
| Compound 21 | —O— | >N— | H | H | H | H | H | H | C₆H₅O | C₆H₅O | H |
| Compound 22 | —O— | >N— | H | H | H | H | H | H | C₆H₅ | C₆H₅ | H |
| Compound 23 | —O— | >N— | H | H | H | H | H | H | CF₃O | CF₃O | H |
| Compound 24 | —O— | >N— | H | H | H | H | H | H | CF₃O | H | H |
| Compound 25 | —S— | >N— | H | H | H | H | H | H | H | H | H |
| Compound 26 | —S— | >N— | H | H | H | H | CH₃O | CH₃O | H | H | H |
| Compound 27 | —S— | >N— | H | H | H | H | CH₃O | H | H | H | H |
| Compound 28 | —S— | >N— | H | H | H | H | CF₃O | CF₃O | H | H | H |
| Compound 29 | —S— | >N— | H | H | H | H | CF₃O | H | H | H | H |
| Compound 30 | —SO₂— | >N— | H | H | H | H | H | H | H | H | H |
| Compound 31 | —SO₂— | >N— | H | H | H | H | CH₃O | CH₃O | H | H | H |
| Compound 32 | —SO₂— | >N— | H | H | H | H | CH₃O | H | H | H | H |
| Compound 33 | —SO₂— | >N— | H | H | H | H | CF₃O | CF₃O | H | H | H |
| Compound 34 | —SO₂— | >N— | H | H | H | H | CF₃O | H | H | H | H |
| Compound 35 | —CH₂— | >N— | H | H | H | H | H | H | H | H | H |
| Compound 36 | —CH₂— | >N— | H | H | H | H | CH₃O | CH₃O | H | H | H |
| Compound 37 | —CH₂— | >N— | H | H | H | H | CH₃O | H | H | H | H |
| Compound 38 | —CH₂— | >N— | H | H | H | H | CF₃O | CF₃O | H | H | H |
| Compound 39 | —CH₂— | >N— | H | H | H | H | CF₃O | H | H | H | H |
| Compound 40 | >C═O | >N— | H | H | H | H | H | H | H | H | H |
| Compound 41 | >C═O | >N— | H | H | H | H | CH₃O | CH₃O | H | H | H |
| Compound 42 | >C═O | >N— | H | H | H | H | CH₃O | H | H | H | H |
| Compound 43 | >C═O | >N— | H | H | H | H | CF₃O | CF₃O | H | H | H |
| Compound 44 | >C═O | >N— | H | H | H | H | CF₃O | H | H | H | H |
| Compound 45 | >C═CH₂ | >N— | H | H | H | H | H | H | H | H | H |
| Compound 46 | >C═CH₂ | >N— | H | H | H | H | CH₃O | CH₃O | H | H | H |
| Compound 47 | >C═CH₂ | >N— | H | H | H | H | CH₃O | H | H | H | H |
| Compound 48 | >C═CH₂ | >N— | H | H | H | H | CF₃O | CF₃O | H | H | H |
| Compound 49 | >C═CH₂ | >N— | H | H | H | H | CF₃O | H | H | H | H |
| Compound 50 | >C═NH | >N— | H | H | H | H | H | H | H | H | H |
| Compound 51 | >C═NH | >N— | H | H | H | H | CH₃O | CH₃O | H | H | H |
| Compound 52 | >C═NH | >N— | H | H | H | H | CH₃O | H | H | H | H |
| Compound 53 | >C═NH | >N— | H | H | H | H | CF₃O | CF₃O | H | H | H |
| Compound 54 | >C═NH | >N— | H | H | H | H | CF₃O | H | H | H | H |
| Compound 55 | >NCH₃ | >N— | H | H | H | H | H | H | H | H | H |
| Compound 56 | >NCH₃ | >N— | H | H | H | H | CH₃O | CH₃O | H | H | H |
| Compound 57 | >NCH₃ | >N— | H | H | H | H | CH₃O | H | H | H | H |
| Compound 58 | >NCH₃ | >N— | H | H | H | H | CF₃O | CF₃O | H | H | H |
| Compound 59 | >NCH₃ | >N— | H | H | H | H | CF₃O | H | H | H | H |
| Compound 60 | —O— | >B— | H | H | H | H | H | H | H | H | H |
| Compound 61 | —O— | >B— | H | H | H | H | CH₃O | CH₃O | H | H | H |
| Compound 62 | —O— | >B— | H | H | H | H | CH₃O | H | H | H | H |
| Compound 63 | —O— | >B— | H | H | H | H | CF₃O | CF₃O | H | H | H |
| Compound 64 | —O— | >B— | H | H | H | H | CF₃O | H | H | H | H |
| Compound 65 | —O— | >P(═O)— | H | H | H | H | H | H | H | H | H |
| Compound 66 | —O— | >P(═O)— | H | H | H | H | CH₃O | CH₃O | H | H | H |
| Compound 67 | —O— | >P(═O)— | H | H | H | H | CH₃O | H | H | H | H |
| Compound 68 | —O— | >P(═O)— | H | H | H | H | CF₃O | CF₃O | H | H | H |
| Compound 69 | —O— | >P(═O)— | H | H | H | H | CF₃O | H | H | H | H |
| Compound 70 | —O— | >N— | —O— | H | H | H | H | H | H | H | H |
| Compound 71 | —O— | >N— | —O— | H | H | H | CH₃O | CH₃O | H | H | H |
| Compound 72 | —O— | >N— | —O— | H | H | H | CH₃O | H | H | H | H |
| Compound 73 | —O— | >N— | —O— | H | H | H | CF₃O | CF₃O | H | H | H |
| Compound 74 | —O— | >N— | —O— | H | H | H | CF₃O | H | H | H | H |
| Compound 75 | —O— | >N— | —O— | —O— | H | H | H | H | H | H | H |
| Compound 76 | —O— | >N— | —O— | —O— | H | H | CH₃O | CH₃O | H | H | H |
| Compound 77 | —O— | >N— | —O— | —O— | H | H | CH₃O | H | H | H | H |
| Compound 78 | —O— | >N— | —O— | —O— | H | H | CF₃O | CF₃O | H | H | H |
| Compound 79 | —O— | >N— | —O— | —O— | H | H | CF₃O | H | H | H | H |
| Compound 80 | —O— | >N— | H | H | CF₃ | H | H | H | H | H | H |
| Compound 81 | —O— | >N— | H | H | H | CF₃ | H | H | H | H | H |
| Compound 82 | —O— | >N— | H | H | H | H | CF₃ | H | H | H | H |
| Compound 83 | —O— | >N— | H | H | H | H | H | CF₃ | H | H | H |
| Compound 84 | —O— | >N— | H | H | H | H | H | H | CF₃ | H | H |
| Compound 85 | —O— | >N— | H | H | H | H | H | H | H | CF₃ | H |
| Compound 86 | —O— | >N— | H | H | CF₃ | CF₃ | H | H | H | H | H |
| Compound 87 | —O— | >N— | H | H | CF₃ | H | CF₃ | H | H | H | H |
| Compound 88 | —O— | >N— | H | H | CF₃ | H | H | CF₃ | H | H | H |
| Compound 89 | —O— | >N— | H | H | CF₃ | H | H | H | CF₃ | H | H |
| Compound 90 | —O— | >N— | H | H | CF₃ | H | H | H | H | CF₃ | H |
| Compound 91 | —O— | >N— | H | H | H | CF₃ | CF₃ | H | H | H | H |
| Compound 92 | —O— | >N— | H | H | H | CF₃ | H | CF₃ | H | H | H |
| Compound 93 | —O— | >N— | H | H | H | CF₃ | H | H | CF₃ | H | H |
| Compound 94 | —O— | >N— | H | H | H | CF₃ | H | H | H | CF₃ | H |
| Compound 95 | —O— | >N— | H | H | H | H | CF₃ | CF₃ | H | H | H |
| Compound 96 | —O— | >N— | H | H | H | H | CF₃ | H | CF₃ | H | H |
| Compound 97 | —O— | >N— | H | H | H | H | CF₃ | H | H | CF₃ | H |
| Compound 98 | —O— | >N— | H | H | H | H | H | CF₃ | CF₃ | H | H |
| Compound 99 | —O— | >N— | H | H | H | H | H | CF₃ | H | CF₃ | H |
| Compound 100 | —O— | >N— | H | H | H | H | H | H | CF₃ | CF₃ | H |

(Note)
In the column of R¹ and R³, —O—means that R¹ and R³ together form —O—.
(Note)
In the column of R² and R⁴, —O—means that R² and R³ together form —O—.

TABLE 2

| | General Formula [2-1] | | | | | | | | | | |
|---|---|---|---|---|---|---|---|---|---|---|---|
| | | | | | | | R⁷, L¹³, | | | R⁵, L¹³, | R⁷, L¹³, |
| | | | | | R⁵, R⁶ | R¹¹, R¹² | L¹⁴, R¹⁰ | R⁶, R⁷ | R¹⁰, R¹¹ | L¹⁴, R¹² | L¹⁴, R¹⁷ |
| Compound | X¹, X², | | | | and | and | R¹⁶, L¹⁷, | and | and | R¹³, L¹⁷, | R¹⁵, L¹⁷, |
| No. | X⁴, X⁵ | Y¹, Y² | R¹, R³ | R², R⁴ | R¹³, R¹⁴ | R¹⁹, R²⁰ | L¹⁸, R¹⁹ | R¹⁴, R¹⁵ | R¹⁸, R¹⁹ | L¹⁸, R²⁰ | L¹⁹, R²⁰ |
| Compound 101 | —O— | >N— | H | H | —CH═CH—CH═CH— | H | H | | | | |
| Compound 102 | —O— | >N— | H | H | —CH═CH—CH═CH— | —CH═CH—CH═CH— | H | | | | |
| Compound 103 | —O— | >N— | H | H | | | | —CH═CH—CH═CH— | H | H | |
| Compound 104 | —O— | >N— | H | H | | | | —CH═CH—CH═CH— | —CH═CH—CH═CH— | H | |
| Compound 105 | —O— | >N— | H | H | —CH═CH—CH═CH— | | | | —CH═CH—CH═CH— | | H |
| Compound 106 | —S— | >N— | H | H | —CH═CH—CH═CH— | H | H | | | | |
| Compound 107 | —S— | >N— | H | H | —CH═CH—CH═CH— | —CH═CH—CH═CH— | H | | | | |
| Compound 108 | —S— | >N— | H | H | | | | —CH═CH—CH═CH— | H | H | |
| Compound 109 | —S— | >N— | H | H | | | | —CH═CH—CH═CH— | —CH═CH—CH═CH— | H | |
| Compound 110 | —S— | >N— | H | H | —CH═CH—CH═CH— | | | | —CH═CH—CH═CH— | | H |
| Compound 111 | —SO₂— | >N— | H | H | —CH═CH—CH═CH— | H | H | | | | |
| Compound 112 | —SO₂— | >N— | H | H | —CH═CH—CH═CH— | —CH═CH—CH═CH— | H | | | | |
| Compound 113 | —SO₂— | >N— | H | H | | | | —CH═CH—CH═CH— | H | H | |
| Compound 114 | —SO₂— | >N— | H | H | | | | —CH═CH—CH═CH— | —CH═CH—CH═CH— | H | |
| Compound 115 | —SO₂— | >N— | H | H | —CH═CH—CH═CH— | | | | —CH═CH—CH═CH— | | H |
| Compound 116 | —CH₂— | >N— | H | H | —CH═CH—CH═CH— | H | H | | | | |
| Compound 117 | —CH₂— | >N— | H | H | —CH═CH—CH═CH— | —CH═CH—CH═CH— | H | | | | |
| Compound 118 | —CH₂— | >N— | H | H | | | | —CH═CH—CH═CH— | H | H | |
| Compound 119 | —CH₂— | >N— | H | H | | | | —CH═CH—CH═CH— | —CH═CH—CH═CH— | H | |
| Compound 120 | —CH₂— | >N— | H | H | —CH═CH—CH═CH— | | | | —CH═CH—CH═CH— | | H |
| Compound 121 | >C═O | >N— | H | H | —CH═CH—CH═CH— | H | H | | | | |
| Compound 122 | >C═O | >N— | H | H | —CH═CH—CH═CH— | —CH═CH—CH═CH— | H | | | | |
| Compound 123 | >C═O | >N— | H | H | | | | —CH═CH—CH═CH— | H | H | |
| Compound 124 | >C═O | >N— | H | H | | | | —CH═CH—CH═CH— | —CH═CH—CH═CH— | H | |
| Compound 125 | >C═O | >N— | H | H | —CH═CH—CH═CH— | | | | —CH═CH—CH═CH— | | H |
| Compound 126 | >C═CH₂ | >N— | H | H | —CH═CH—CH═CH— | H | H | | | | |
| Compound 127 | >C═CH₂ | >N— | H | H | —CH═CH—CH═CH— | —CH═CH—CH═CH— | H | | | | |
| Compound 128 | >C═CH₂ | >N— | H | H | | | | —CH═CH—CH═CH— | H | H | |
| Compound 129 | >C═CH₂ | >N— | H | H | | | | —CH═CH—CH═CH— | —CH═CH—CH═CH— | H | |
| Compound 130 | >C═CH₂ | >N— | H | H | —CH═CH—CH═CH— | | | | —CH═CH—CH═CH— | | H |
| Compound 131 | >C═NH | >N— | H | H | —CH═CH—CH═CH— | H | H | | | | |
| Compound 132 | >C═NH | >N— | H | H | —CH═CH—CH═CH— | —CH═CH—CH═CH— | H | | | | |
| Compound 133 | >C═NH | >N— | H | H | | | | —CH═CH—CH═CH— | H | H | |
| Compound 134 | >C═NH | >N— | H | H | | | | —CH═CH—CH═CH— | —CH═CH—CH═CH— | H | |
| Compound 135 | >C═NH | >N— | H | H | —CH═CH—CH═CH— | | | | —CH═CH—CH═CH— | | H |
| Compound 136 | >NCH₃ | >N— | H | H | —CH═CH—CH═CH— | H | H | | | | |
| Compound 137 | >NCH₃ | >N— | H | H | —CH═CH—CH═CH— | —CH═CH—CH═CH— | H | | | | |
| Compound 138 | >NCH₃ | >N— | H | H | | | | —CH═CH—CH═CH— | H | H | |
| Compound 139 | >NCH₃ | >N— | H | H | | | | —CH═CH—CH═CH— | —CH═CH—CH═CH— | H | |
| Compound 140 | >NCH₃ | >N— | H | H | —CH═CH—CH═CH— | | | | —CH═CH—CH═CH— | | H |
| Compound 141 | —O— | >B— | H | H | —CH═CH—CH═CH— | H | H | | | | |
| Compound 142 | —O— | >B— | H | H | —CH═CH—CH═CH— | —CH═CH—CH═CH— | H | | | | |
| Compound 143 | —O— | >B— | H | H | | | | —CH═CH—CH═CH— | H | H | |
| Compound 144 | —O— | >B— | H | H | | | | —CH═CH—CH═CH— | —CH═CH—CH═CH— | H | |
| Compound 145 | —O— | >B— | H | H | —CH═CH—CH═CH— | | | | —CH═CH—CH═CH— | | H |
| Compound 146 | —O— | >P(═O)— | H | H | —CH═CH—CH═CH— | H | H | | | | |
| Compound 147 | —O— | >P(═O)— | H | H | —CH═CH—CH═CH— | —CH═CH—CH═CH— | H | | | | |
| Compound 148 | —O— | >P(═O)— | H | H | | | | —CH═CH—CH═CH— | H | H | |
| Compound 149 | —O— | >P(═O)— | H | H | | | | —CH═CH—CH═CH— | —CH═CH—CH═CH— | H | |
| Compound 150 | —O— | >P(═O)— | H | H | —CH═CH—CH═CH— | | | | —CH═CH—CH═CH— | | H |
| Compound 151 | —O— | >N— | —O— | H | —CH═CH—CH═CH— | H | H | | | | |
| Compound 152 | —O— | >N— | —O— | H | —CH═CH—CH═CH— | —CH═CH—CH═CH— | H | | | | |
| Compound 153 | —O— | >N— | —O— | H | | | | —CH═CH—CH═CH— | H | H | |
| Compound 154 | —O— | >N— | —O— | H | | | | —CH═CH—CH═CH— | —CH═CH—CH═CH— | H | |
| Compound 155 | —O— | >N— | —O— | H | —CH═CH—CH═CH— | | | | —CH═CH—CH═CH— | | H |
| Compound 156 | —O— | >N— | —O— | —O— | —CH═CH—CH═CH— | H | H | | | | |
| Compound 157 | —O— | >N— | —O— | —O— | —CH═CH—CH═CH— | —CH═CH—CH═CH— | H | | | | |
| Compound 158 | —O— | >N— | —O— | —O— | | | | —CH═CH—CH═CH— | H | H | |
| Compound 159 | —O— | >N— | —O— | —O— | | | | —CH═CH—CH═CH— | —CH═CH—CH═CH— | H | |
| Compound 160 | —O— | >N— | —O— | —O— | —CH═CH—CH═CH— | | | | —CH═CH—CH═CH— | | H |

(Note)
In the column of R¹ and R³, —O—means that R¹ and R³ together form —O—.
(Note)
In the column of R² and R⁴, —O—means that R² and R⁴ together form —O—.

TABLE 3

| Compound | General Formula [2-2] | | | | | | | | | | |
|---|---|---|---|---|---|---|---|---|---|---|---|
| No. | X¹, X², X⁴, X⁶ | Y¹, Y² | R¹, R³ | R², R⁴ | R³, R¹³ | R⁴, R¹⁴ | R⁷, R¹⁵ | R¹⁰, R¹⁶ | R¹¹, R¹⁸ | R¹², R²⁰ | L¹¹, L¹², L¹⁵, L¹⁶ |
| Compound 201 | —O— | >N— | H | H | H | H | H | H | H | H | H |
| Compound 202 | —O— | >N— | H | H | H | H | CH₃O | CH₃O | H | H | H |
| Compound 203 | —O— | >N— | H | H | H | H | CH₃O | H | H | H | H |
| Compound 204 | —O— | >N— | H | H | H | H | CH₃ | H | H | H | H |
| Compound 205 | —O— | >N— | H | H | H | H | C₆H₅O | C₆H₅O | H | H | H |
| Compound 206 | —O— | >N— | H | H | H | H | C₆H₅ | C₆H₅ | H | H | H |
| Compound 207 | —O— | >N— | H | H | H | H | CF₃O | CF₃O | H | H | H |
| Compound 208 | —O— | >N— | H | H | H | H | CF₃O | H | H | H | H |
| Compound 209 | —O— | >N— | H | H | H | H | H | H | H | H | H |
| Compound 210 | —O— | >N— | H | H | CH₃O | CH₃O | H | H | H | H | H |
| Compound 211 | —O— | >N— | H | H | CH₃O | H | H | H | H | H | H |
| Compound 212 | —O— | >N— | H | H | CH₃ | H | H | H | H | H | H |
| Compound 213 | —O— | >N— | H | H | C₆H₅O | C₆H₅O | H | H | H | H | H |
| Compound 214 | —O— | >N— | H | H | C₆H₅ | C₆H₅ | H | H | H | H | H |
| Compound 215 | —O— | >N— | H | H | CF₃O | CF₃O | H | H | H | H | H |
| Compound 216 | —O— | >N— | H | H | CF₃O | H | H | H | H | H | H |
| Compound 217 | —O— | >N— | H | H | H | H | H | H | H | H | H |
| Compound 218 | —O— | >N— | H | H | H | H | H | H | CH₃O | CH₃O | H |
| Compound 219 | —O— | >N— | H | H | H | H | H | H | CH₃O | H | H |
| Compound 220 | —O— | >N— | H | H | H | H | H | H | CH₃ | H | H |
| Compound 221 | —O— | >N— | H | H | H | H | H | H | C₆H₅O | C₆H₅O | H |
| Compound 222 | —O— | >N— | H | H | H | H | H | H | C₆H₅ | C₆H₅ | H |
| Compound 223 | —O— | >N— | H | H | H | H | H | H | CF₃O | CF₃O | H |
| Compound 224 | —O— | >N— | H | H | H | H | H | H | CF₃O | H | H |
| Compound 225 | —S— | >N— | H | H | H | H | H | H | H | H | H |
| Compound 226 | —S— | >N— | H | H | H | H | CH₃O | CH₃O | H | H | H |
| Compound 227 | —S— | >N— | H | H | H | H | CH₃O | H | H | H | H |
| Compound 228 | —S— | >N— | H | H | H | H | CF₃O | CF₃O | H | H | H |
| Compound 229 | —S— | >N— | H | H | H | H | CF₃O | H | H | H | H |
| Compound 230 | —SO₂— | >N— | H | H | H | H | H | H | H | H | H |
| Compound 231 | —SO₂— | >N— | H | H | H | H | CH₃O | CH₃O | H | H | H |
| Compound 232 | —SO₂— | >N— | H | H | H | H | CH₃O | H | H | H | H |
| Compound 233 | —SO₂— | >N— | H | H | H | H | CF₃O | CF₃O | H | H | H |
| Compound 234 | —SO₂— | >N— | H | H | H | H | CF₃O | H | H | H | H |
| Compound 235 | —CH₂— | >N— | H | H | H | H | H | H | H | H | H |
| Compound 236 | —CH₂— | >N— | H | H | H | H | CH₃O | CH₃O | H | H | H |
| Compound 237 | —CH₂— | >N— | H | H | H | H | CH₃O | H | H | H | H |
| Compound 238 | —CH₂— | >N— | H | H | H | H | CF₃O | CF₃O | H | H | H |
| Compound 239 | —CH₂— | >N— | H | H | H | H | CF₃O | H | H | H | H |
| Compound 240 | >C═O | >N— | H | H | H | H | H | H | H | H | H |
| Compound 241 | >C═O | >N— | H | H | H | H | CH₃O | CH₃O | H | H | H |
| Compound 242 | >C═O | >N— | H | H | H | H | CH₃O | H | H | H | H |
| Compound 243 | >C═O | >N— | H | H | H | H | CF₃O | CF₃O | H | H | H |
| Compound 244 | >C═O | >N— | H | H | H | H | CF₃O | H | H | H | H |
| Compound 245 | >C═CH₂ | >N— | H | H | H | H | H | H | H | H | H |
| Compound 246 | >C═CH₂ | >N— | H | H | H | H | CH₃O | CH₃O | H | H | H |
| Compound 247 | >C═CH₂ | >N— | H | H | H | H | CH₃O | H | H | H | H |
| Compound 248 | >C═CH₂ | >N— | H | H | H | H | CF₃O | CF₃O | H | H | H |
| Compound 249 | >C═CH₂ | >N— | H | H | H | H | CF₃O | H | H | H | H |
| Compound 250 | >C═NH | >N— | H | H | H | H | H | H | H | H | H |
| Compound 251 | >C═NH | >N— | H | H | H | H | CH₃O | CH₃O | H | H | H |
| Compound 252 | >C═NH | >N— | H | H | H | H | CH₃O | H | H | H | H |
| Compound 253 | >C═NH | >N— | H | H | H | H | CF₃O | CF₃O | H | H | H |
| Compound 254 | >C═NH | >N— | H | H | H | H | CF₃O | H | H | H | H |
| Compound 255 | >NCH₃ | >N— | H | H | H | H | H | H | H | H | H |
| Compound 256 | >NCH₃ | >N— | H | H | H | H | CH₃O | CH₃O | H | H | H |
| Compound 257 | >NCH₃ | >N— | H | H | H | H | CH₃O | H | H | H | H |
| Compound 258 | >NCH₃ | >N— | H | H | H | H | CF₃O | CF₃O | H | H | H |
| Compound 259 | >NCH₃ | >N— | H | H | H | H | CF₃O | H | H | H | H |
| Compound 260 | —O— | >B— | H | H | H | H | H | H | H | H | H |
| Compound 261 | —O— | >B— | H | H | H | H | CH₃O | CH₃O | H | H | H |
| Compound 262 | —O— | >B— | H | H | H | H | CH₃O | H | H | H | H |
| Compound 263 | —O— | >B— | H | H | H | H | CF₃O | CF₃O | H | H | H |
| Compound 264 | —O— | >B— | H | H | H | H | CF₃O | H | H | H | H |
| Compound 265 | —O— | >P(═O)— | H | H | H | H | H | H | H | H | H |
| Compound 266 | —O— | >P(═O)— | H | H | H | H | CH₃O | CH₃O | H | H | H |
| Compound 267 | —O— | >P(═O)— | H | H | H | H | CH₃O | H | H | H | H |
| Compound 268 | —O— | >P(═O)— | H | H | H | H | CF₃O | CF₃O | H | H | H |
| Compound 269 | —O— | >P(═O)— | H | H | H | H | CF₃O | H | H | H | H |
| Compound 270 | —O— | >N— | —O— | H | H | H | H | H | H | H | H |
| Compound 271 | —O— | >N— | —O— | H | H | H | CH₃O | CH₃O | H | H | H |
| Compound 272 | —O— | >N— | —O— | H | H | H | CH₃O | H | H | H | H |
| Compound 273 | —O— | >N— | —O— | H | H | H | CF₃O | CF₃O | H | H | H |
| Compound 274 | —O— | >N— | —O— | H | H | H | CF₃O | H | H | H | H |
| Compound 275 | —O— | >N— | —O— | —O— | H | H | H | H | H | H | H |
| Compound 276 | —O— | >N— | —O— | —O— | H | H | CH₃O | CH₃O | H | H | H |
| Compound 277 | —O— | >N— | —O— | —O— | H | H | CH₃O | H | H | H | H |
| Compound 278 | —O— | >N— | —O— | —O— | H | H | CF₃O | CF₃O | H | H | H |
| Compound 279 | —O— | >N— | —O— | —O— | H | H | CF₃O | H | H | H | H |
| Compound 280 | —O— | >N— | H | H | CF₃ | H | H | H | H | H | H |
| Compound 281 | —O— | >N— | H | H | H | CF₃ | H | H | H | H | H |
| Compound 282 | —O— | >N— | H | H | H | H | CF₃ | H | H | H | H |
| Compound 283 | —O— | >N— | H | H | H | H | H | CF₃ | H | H | H |
| Compound 284 | —O— | >N— | H | H | H | H | H | H | CF₃ | H | H |
| Compound 285 | —O— | >N— | H | H | H | H | H | H | H | CF₃ | H |
| Compound 286 | —O— | >N— | H | H | CF₃ | CF₃ | H | H | H | H | H |
| Compound 287 | —O— | >N— | H | H | CF₃ | H | CF₃ | H | H | H | H |
| Compound 288 | —O— | >N— | H | H | CF₃ | H | H | CF₃ | H | H | H |
| Compound 289 | —O— | >N— | H | H | CF₃ | H | H | H | CF₃ | H | H |
| Compound 290 | —O— | >N— | H | H | CF₃ | H | H | H | H | CF₃ | H |
| Compound 291 | —O— | >N— | H | H | H | CF₃ | CF₃ | H | H | H | H |
| Compound 292 | —O— | >N— | H | H | H | CF₃ | H | CF₃ | H | H | H |
| Compound 293 | —O— | >N— | H | H | H | CF₃ | H | H | CF₃ | H | H |
| Compound 294 | —O— | >N— | H | H | H | CF₃ | H | H | H | CF₃ | H |
| Compound 295 | —O— | >N— | H | H | H | H | CF₃ | CF₃ | H | H | H |
| Compound 296 | —O— | >N— | H | H | H | H | CF₃ | H | CF₃ | H | H |
| Compound 297 | —O— | >N— | H | H | H | H | CF₃ | H | H | CF₃ | H |
| Compound 298 | —O— | >N— | H | H | H | H | H | CF₃ | CF₃ | H | H |
| Compound 299 | —O— | >N— | H | H | H | H | H | CF₃ | H | CF₃ | H |
| Compound 300 | —O— | >N— | H | H | H | H | H | H | CF₃ | CF₃ | H |

(Note)
In the column of R¹ and R³, —O—means that R¹ and R³ together form —O—.
(Note)
In the column of R² and R⁴, —O—means that R² and R⁴ together form —O—.

TABLE 4

| | General Formula (2-2) | | | | | | | | | | |
|---|---|---|---|---|---|---|---|---|---|---|---|
| | | | | | | | L¹¹L¹², | | | L¹¹, L¹², | L¹¹, L¹², |
| | | | | | R⁵, R⁶ | R¹¹, R¹² | R⁷, R¹⁰ | R⁶, R⁷ | R¹⁰, R¹¹ | R⁶, R¹² | R⁷, R¹² |
| Compound | X¹, X³, | | | | and | and | L¹⁵, L¹⁸, | and | and | L¹⁵, L¹⁶, | L¹⁵, L¹⁶, |
| No. | X⁴, X⁶ | Y¹, Y² | R¹, R³ | R², R⁴ | R¹³, R¹⁴ | R¹⁹, R²⁰ | R¹⁵, R¹⁶ | R¹⁴, R¹⁵ | R¹⁸, R¹⁹ | R¹⁸, R²⁰ | R¹⁵, R²⁰ |
| Compound 301 | —O— | >N— | H | H | —CH═CH—CH═CH— | H | H | | | | |
| Compound 302 | —O— | >N— | H | H | —CH═CH—CH═CH— | —CH═CH—CH═CH— | H | | | | |
| Compound 303 | —O— | >N— | H | H | | | | —CH═CH—CH═CH— | H | H | |
| Compound 304 | —O— | >N— | H | H | | | | —CH═CH—CH═CH— | —CH═CH—CH═CH— | H | |
| Compound 305 | —O— | >N— | H | H | —CH═CH—CH═CH— | | | | —CH═CH—CH═CH— | | H |
| Compound 306 | —S— | >N— | H | H | —CH═CH—CH═CH— | H | H | | | | |
| Compound 307 | —S— | >N— | H | H | —CH═CH—CH═CH— | —CH═CH—CH═CH— | H | | | | |
| Compound 308 | —S— | >N— | H | H | | | | —CH═CH—CH═CH— | H | H | |
| Compound 309 | —S— | >N— | H | H | | | | —CH═CH—CH═CH— | —CH═CH—CH═CH— | H | |
| Compound 310 | —S— | >N— | H | H | —CH═CH—CH═CH— | | | | —CH═CH—CH═CH— | | H |
| Compound 311 | —SO₂— | >N— | H | H | —CH═CH—CH═CH— | H | H | | | | |
| Compound 312 | —SO₂— | >N— | H | H | —CH═CH—CH═CH— | —CH═CH—CH═CH— | H | | | | |
| Compound 313 | —SO₂— | >N— | H | H | | | | —CH═CH—CH═CH— | H | H | |
| Compound 314 | —SO₂— | >N— | H | H | | | | —CH═CH—CH═CH— | —CH═CH—CH═CH— | H | |
| Compound 315 | —SO₂— | >N— | H | H | —CH═CH—CH═CH— | | | | —CH═CH—CH═CH— | | H |
| Compound 316 | —CH₂— | >N— | H | H | —CH═CH—CH═CH— | H | H | | | | |
| Compound 317 | —CH₂— | >N— | H | H | —CH═CH—CH═CH— | —CH═CH—CH═CH— | H | | | | |
| Compound 318 | —CH₂— | >N— | H | H | | | | —CH═CH—CH═CH— | H | H | |
| Compound 319 | —CH₂— | >N— | H | H | | | | —CH═CH—CH═CH— | —CH═CH—CH═CH— | H | |
| Compound 320 | —CH₂— | >N— | H | H | —CH═CH—CH═CH— | | | | —CH═CH—CH═CH— | | H |
| Compound 321 | >C═O | >N— | H | H | —CH═CH—CH═CH— | H | H | | | | |
| Compound 322 | >C═O | >N— | H | H | —CH═CH—CH═CH— | —CH═CH—CH═CH— | H | | | | |
| Compound 323 | >C═O | >N— | H | H | | | | —CH═CH—CH═CH— | H | H | |
| Compound 324 | >C═O | >N— | H | H | | | | —CH═CH—CH═CH— | —CH═CH—CH═CH— | H | |
| Compound 325 | >C═O | >N— | H | H | —CH═CH—CH═CH— | | | | —CH═CH—CH═CH— | | H |
| Compound 326 | >C═CH₂ | >N— | H | H | —CH═CH—CH═CH— | H | H | | | | |
| Compound 327 | >C═CH₂ | >N— | H | H | —CH═CH—CH═CH— | —CH═CH—CH═CH— | H | | | | |
| Compound 328 | >C═CH₂ | >N— | H | H | | | | —CH═CH—CH═CH— | H | H | |
| Compound 329 | >C═CH₂ | >N— | H | H | | | | —CH═CH—CH═CH— | —CH═CH—CH═CH— | H | |
| Compound 330 | >C═CH₂ | >N— | H | H | —CH═CH—CH═CH— | | | | —CH═CH—CH═CH— | | H |
| Compound 331 | >C═NH | >N— | H | H | —CH═CH—CH═CH— | H | H | | | | |
| Compound 332 | >C═NH | >N— | H | H | —CH═CH—CH═CH— | —CH═CH—CH═CH— | H | | | | |
| Compound 333 | >C═NH | >N— | H | H | | | | —CH═CH—CH═CH— | H | H | |
| Compound 334 | >C═NH | >N— | H | H | | | | —CH═CH—CH═CH— | —CH═CH—CH═CH— | H | |
| Compound 335 | >C═NH | >N— | H | H | —CH═CH—CH═CH— | | | | —CH═CH—CH═CH— | | H |
| Compound 336 | >NCH₃ | >N— | H | H | —CH═CH—CH═CH— | H | H | | | | |
| Compound 337 | >NCH₃ | >N— | H | H | —CH═CH—CH═CH— | —CH═CH—CH═CH— | H | | | | |
| Compound 338 | >NCH₃ | >N— | H | H | | | | —CH═CH—CH═CH— | H | H | |
| Compound 339 | >NCH₃ | >N— | H | H | | | | —CH═CH—CH═CH— | —CH═CH—CH═CH— | H | |
| Compound 340 | >NCH₃ | >N— | H | H | —CH═CH—CH═CH— | | | | —CH═CH—CH═CH— | | H |
| Compound 341 | —O— | >B— | H | H | —CH═CH—CH═CH— | H | H | | | | |
| Compound 342 | —O— | >B— | H | H | —CH═CH—CH═CH— | —CH═CH—CH═CH— | H | | | | |
| Compound 343 | —O— | >B— | H | H | | | | —CH═CH—CH═CH— | H | H | |
| Compound 344 | —O— | >B— | H | H | | | | —CH═CH—CH═CH— | —CH═CH—CH═CH— | H | |
| Compound 345 | —O— | >B— | H | H | —CH═CH—CH═CH— | | | | —CH═CH—CH═CH— | | H |
| Compound 346 | —O— | >P(═O)— | H | H | —CH═CH—CH═CH— | H | H | | | | |
| Compound 347 | —O— | >P(═O)— | H | H | —CH═CH—CH═CH— | —CH═CH—CH═CH— | H | | | | |
| Compound 348 | —O— | >P(═O)— | H | H | | | | —CH═CH—CH═CH— | H | H | |
| Compound 349 | —O— | >P(═O)— | H | H | | | | —CH═CH—CH═CH— | —CH═CH—CH═CH— | H | |
| Compound 350 | —O— | >P(═O)— | H | H | —CH═CH—CH═CH— | | | | —CH═CH—CH═CH— | | H |
| Compound 351 | —O— | >N— | —O— | H | —CH═CH—CH═CH— | H | H | | | | |
| Compound 352 | —O— | >N— | —O— | H | —CH═CH—CH═CH— | —CH═CH—CH═CH— | H | | | | |
| Compound 353 | —O— | >N— | —O— | H | | | | —CH═CH—CH═CH— | H | H | |
| Compound 354 | —O— | >N— | —O— | H | | | | —CH═CH—CH═CH— | —CH═CH—CH═CH— | H | |
| Compound 355 | —O— | >N— | —O— | H | —CH═CH—CH═CH— | | | | —CH═CH—CH═CH— | | H |
| Compound 356 | —O— | >N— | —O— | —O— | —CH═CH—CH═CH— | H | H | | | | |
| Compound 357 | —O— | >N— | —O— | —O— | —CH═CH—CH═CH— | —CH═CH—CH═CH— | H | | | | |
| Compound 358 | —O— | >N— | —O— | —O— | | | | —CH═CH—CH═CH— | H | H | |
| Compound 359 | —O— | >N— | —O— | —O— | | | | —CH═CH—CH═CH— | —CH═CH—CH═CH— | H | |
| Compound 360 | —O— | >N— | —O— | —O— | —CH═CH—CH═CH— | | | | —CH═CH—CH═CH— | | H |

(Note)
In the column of R¹ and R³, —O—means that R¹ and R³ together form —O—.
(Note)
In the column of R² and R⁴, —O—means that R² and R⁴ together form —O—.

TABLE 5

| | General Formula (5) | | | | | | | | | | | |
|---|---|---|---|---|---|---|---|---|---|---|---|---|
| Compound | | L¹ and L², | L³ and L⁴, | | | | | | | | | |
| No. | X¹, X⁴ | L⁵ and L⁶ | L⁷ and L⁸ | Y¹, Y² | R¹, R⁴ | R², R³ | R⁶, R¹³ | R⁶, R¹⁴ | R⁷, R¹⁵ | R¹⁶, R¹⁸ | R¹¹, R¹⁹ | R¹², R²⁰ |
| Compound 401 | —O— | —O— | H | >N— | H | H | H | H | H | H | H | H |
| Compound 402 | —O— | —O— | H | >N— | H | H | H | H | CH₃O | CH₃O | H | H |
| Compound 403 | —O— | —O— | H | >N— | H | H | H | H | CH₃O | H | H | H |
| Compound 404 | —O— | —O— | H | >N— | H | H | H | H | CH₃ | H | H | H |
| Compound 405 | —O— | —O— | H | >N— | H | H | H | H | C₆H₅O | C₆H₅O | H | H |
| Compound 406 | —O— | —O— | H | >N— | H | H | H | H | C₆H₅ | C₆H₅ | H | H |
| Compound 407 | —O— | —O— | H | >N— | H | H | H | H | CF₃O | CF₃O | H | H |
| Compound 408 | —O— | —O— | H | >N— | H | H | H | H | CF₃O | H | H | H |
| Compound 409 | —O— | —O— | H | >N— | H | H | H | H | H | H | H | H |
| Compound 410 | —O— | —O— | H | >N— | H | H | CH₃O | CH₃O | H | H | H | H |
| Compound 411 | —O— | —O— | H | >N— | H | H | CH₃O | H | H | H | H | H |
| Compound 412 | —O— | —O— | H | >N— | H | H | CH₃ | H | H | H | H | H |
| Compound 413 | —O— | —O— | H | >N— | H | H | C₆H₅O | C₆H₅O | H | H | H | H |
| Compound 414 | —O— | —O— | H | >N— | H | H | C₆H₅ | C₆H₅ | H | H | H | H |
| Compound 415 | —O— | —O— | H | >N— | H | H | CF₃O | CF₃O | H | H | H | H |
| Compound 416 | —O— | —O— | H | >N— | H | H | CF₃O | H | H | H | H | H |
| Compound 417 | —O— | —O— | H | >N— | H | H | H | H | H | H | H | H |
| Compound 418 | —O— | —O— | H | >N— | H | H | H | H | H | H | CH₃O | CH₃O |
| Compound 419 | —O— | —O— | H | >N— | H | H | H | H | H | H | CH₃O | H |
| Compound 420 | —O— | —O— | H | >N— | H | H | H | H | H | H | CH₃ | H |
| Compound 421 | —O— | —O— | H | >N— | H | H | H | H | H | H | C₆H₅O | C₆H₅O |
| Compound 422 | —O— | —O— | H | >N— | H | H | H | H | H | H | C₆H₅ | C₆H₅ |
| Compound 423 | —O— | —O— | H | >N— | H | H | H | H | H | H | CF₃O | CF₃O |
| Compound 424 | —O— | —O— | H | >N— | H | H | H | H | H | H | CF₃O | H |
| Compound 425 | —S— | —S— | H | >N— | H | H | H | H | H | H | H | H |
| Compound 426 | —S— | —S— | H | >N— | H | H | H | H | CH₃O | CH₃O | H | H |
| Compound 427 | —S— | —S— | H | >N— | H | H | H | H | CH₃O | H | H | H |
| Compound 428 | —S— | —S— | H | >N— | H | H | H | H | CF₃O | CF₃O | H | H |
| Compound 429 | —S— | —S— | H | >N— | H | H | H | H | CF₃O | H | H | H |
| Compound 430 | —SO₂— | —SO₂— | H | >N— | H | H | H | H | H | H | H | H |
| Compound 431 | —SO₂— | —SO₂— | H | >N— | H | H | H | H | CH₃O | CH₃O | H | H |
| Compound 432 | —SO₂— | —SO₂— | H | >N— | H | H | H | H | CH₃O | H | H | H |
| Compound 433 | —SO₂— | —SO₂— | H | >N— | H | H | H | H | CF₃O | CF₃O | H | H |
| Compound 434 | —SO₂— | —SO₂— | H | >N— | H | H | H | H | CF₃O | H | H | H |
| Compound 435 | —CH₂— | —CH₂— | H | >N— | H | H | H | H | H | H | H | H |
| Compound 436 | —CH₂— | —CH₂— | H | >N— | H | H | H | H | CH₃O | CH₃O | H | H |
| Compound 437 | —CH₂— | —CH₂— | H | >N— | H | H | H | H | CH₃O | H | H | H |
| Compound 438 | —CH₂— | —CH₂— | H | >N— | H | H | H | H | CF₃O | CF₃O | H | H |
| Compound 439 | —CH₂— | —CH₂— | H | >N— | H | H | H | H | CF₃O | H | H | H |
| Compound 440 | >C═O | >C═O | H | >N— | H | H | H | H | H | H | H | H |
| Compound 441 | >C═O | >C═O | H | >N— | H | H | H | H | CH₃O | CH₃O | H | H |
| Compound 442 | >C═O | >C═O | H | >N— | H | H | H | H | CH₃O | H | H | H |
| Compound 443 | >C═O | >C═O | H | >N— | H | H | H | H | CF₃O | CF₃O | H | H |
| Compound 444 | >C═O | >C═O | H | >N— | H | H | H | H | CF₃O | H | H | H |
| Compound 445 | >C═CH₂ | >C═CH₂ | H | >N— | H | H | H | H | H | H | H | H |
| Compound 446 | >C═CH₂ | >C═CH₂ | H | >N— | H | H | H | H | CH₃O | CH₃O | H | H |
| Compound 447 | >C═CH₂ | >C═CH₂ | H | >N— | H | H | H | H | CH₃O | H | H | H |
| Compound 448 | >C═CH₂ | >C═CH₂ | H | >N— | H | H | H | H | CF₃O | CF₃O | H | H |
| Compound 449 | >C═CH₂ | >C═CH₂ | H | >N— | H | H | H | H | CF₃O | H | H | H |
| Compound 450 | >C═NH | >C═NH | H | >N— | H | H | H | H | H | H | H | H |
| Compound 451 | >C═NH | >C═NH | H | >N— | H | H | H | H | CH₃O | CH₃O | H | H |
| Compound 452 | >C═NH | >C═NH | H | >N— | H | H | H | H | CH₃O | H | H | H |
| Compound 453 | >C═NH | >C═NH | H | >N— | H | H | H | H | CF₃O | CF₃O | H | H |
| Compound 454 | >C═NH | >C═NH | H | >N— | H | H | H | H | CF₃O | H | H | H |
| Compound 455 | >NCH₃ | >NCH₃ | H | >N— | H | H | H | H | H | H | H | H |
| Compound 456 | >NCH₃ | >NCH₃ | H | >N— | H | H | H | H | CH₃O | CH₃O | H | H |
| Compound 457 | >NCH₃ | >NCH₃ | H | >N— | H | H | H | H | CH₃O | H | H | H |
| Compound 458 | >NCH₃ | >NCH₃ | H | >N— | H | H | H | H | CF₃O | CF₃O | H | H |
| Compound 459 | >NCH₃ | >NCH₃ | H | >N— | H | H | H | H | CF₃O | H | H | H |
| Compound 460 | —O— | —O— | H | >B— | H | H | H | H | H | H | H | H |
| Compound 461 | —O— | —O— | H | >B— | H | H | H | H | CH₃O | CH₃O | H | H |
| Compound 462 | —O— | —O— | H | >B— | H | H | H | H | CH₃O | H | H | H |
| Compound 463 | —O— | —O— | H | >B— | H | H | H | H | CF₃O | CF₃O | H | H |
| Compound 464 | —O— | —O— | H | >B— | H | H | H | H | CF₃O | H | H | H |
| Compound 465 | —O— | —O— | H | >P(═O)— | H | H | H | H | H | H | H | H |
| Compound 466 | —O— | —O— | H | >P(═O)— | H | H | H | H | CH₃O | CH₃O | H | H |
| Compound 467 | —O— | —O— | H | >P(═O)— | H | H | H | H | CH₃O | H | H | H |
| Compound 468 | —O— | —O— | H | >P(═O)— | H | H | H | H | CF₃O | CF₃O | H | H |
| Compound 469 | —O— | —O— | H | >P(═O)— | H | H | H | H | CF₃O | H | H | H |
| Compound 470 | —O— | H | —O— | >N— | H | H | H | H | H | H | H | H |
| Compound 471 | —O— | H | —O— | >N— | H | H | H | H | CH₃O | CH₃O | H | H |
| Compound 472 | —O— | H | —O— | >N— | H | H | H | H | CH₃O | H | H | H |
| Compound 473 | —O— | H | —O— | >N— | H | H | H | H | CH₃ | H | H | H |
| Compound 474 | —O— | H | —O— | >N— | H | H | H | H | C₆H₅O | C₆H₅O | H | H |
| Compound 475 | —O— | H | —O— | >N— | H | H | H | H | C₆H₅ | C₆H₅ | H | H |
| Compound 476 | —O— | H | —O— | >N— | H | H | H | H | CF₃O | CF₃O | H | H |
| Compound 477 | —O— | H | —O— | >N— | H | H | H | H | CF₃O | H | H | H |
| Compound 478 | —O— | H | —O— | >N— | H | H | H | H | H | H | H | H |
| Compound 479 | —O— | H | —O— | >N— | H | H | CH₃O | CH₃O | H | H | H | H |
| Compound 480 | —O— | H | —O— | >N— | H | H | CH₃O | H | H | H | H | H |
| Compound 481 | —O— | H | —O— | >N— | H | H | CH₃ | H | H | H | H | H |
| Compound 482 | —O— | H | —O— | >N— | H | H | C₆H₅O | C₆H₅O | H | H | H | H |
| Compound 483 | —O— | H | —O— | >N— | H | H | C₆H₅ | C₆H₅ | H | H | H | H |
| Compound 484 | —O— | H | —O— | >N— | H | H | CF₃O | CF₃O | H | H | H | H |
| Compound 485 | —O— | H | —O— | >N— | H | H | CF₃O | H | H | H | H | H |
| Compound 486 | —O— | H | —O— | >N— | H | H | H | H | H | H | H | H |
| Compound 487 | —O— | H | —O— | >N— | H | H | H | H | H | H | CH₃O | CH₃O |
| Compound 488 | —O— | H | —O— | >N— | H | H | H | H | H | H | CH₃O | H |
| Compound 489 | —O— | H | —O— | >N— | H | H | H | H | H | H | CH₃ | H |
| Compound 490 | —O— | H | —O— | >N— | H | H | H | H | H | H | C₆H₅O | C₆H₅O |
| Compound 491 | —O— | H | —O— | >N— | H | H | H | H | H | H | C₆H₅ | C₆H₅ |
| Compound 492 | —O— | H | —O— | >N— | H | H | H | H | H | H | CF₃O | CF₃O |
| Compound 493 | —O— | H | —O— | >N— | H | H | H | H | H | H | CF₃O | H |
| Compound 494 | —S— | H | —S— | >N— | H | H | H | H | H | H | H | H |
| Compound 495 | —S— | H | —S— | >N— | H | H | H | H | CH₃O | CH₃O | H | H |
| Compound 496 | —S— | H | —S— | >N— | H | H | H | H | CH₃O | H | H | H |
| Compound 497 | —S— | H | —S— | >N— | H | H | H | H | CF₃O | CF₃O | H | H |
| Compound 498 | —S— | H | —S— | >N— | H | H | H | H | CF₃O | H | H | H |
| Compound 499 | —SO₂— | H | —SO₂— | >N— | H | H | H | H | H | H | H | H |
| Compound 500 | —SO₂— | H | —SO₂— | >N— | H | H | H | H | CH₃O | CH₃O | H | H |
| Compound 501 | —SO₂— | H | —SO₂— | >N— | H | H | H | H | CH₃O | H | H | H |
| Compound 502 | —SO₂— | H | —SO₂— | >N— | H | H | H | H | CF₃O | CF₃O | H | H |
| Compound 503 | —SO₂— | H | —SO₂— | >N— | H | H | H | H | CF₃O | H | H | H |
| Compound 504 | —CH₂— | H | —CH₂— | >N— | H | H | H | H | H | H | H | H |
| Compound 505 | —CH₂— | H | —CH₂— | >N— | H | H | H | H | CH₃O | CH₃O | H | H |
| Compound 506 | —CH₂— | H | —CH₂— | >N— | H | H | H | H | CH₃O | H | H | H |
| Compound 507 | —CH₂— | H | —CH₂— | >N— | H | H | H | H | CF₃O | CF₃O | H | H |
| Compound 508 | —CH₂— | H | —CH₂— | >N— | H | H | H | H | CF₃O | H | H | H |
| Compound 509 | >C═O | H | >C═O | >N— | H | H | H | H | H | H | H | H |
| Compound 510 | >C═O | H | >C═O | >N— | H | H | H | H | CH₃O | CH₃O | H | H |
| Compound 511 | >C═O | H | >C═O | >N— | H | H | H | H | CH₃O | H | H | H |
| Compound 512 | >C═O | H | >C═O | >N— | H | H | H | H | CF₃O | CF₃O | H | H |
| Compound 513 | >C═O | H | >C═O | >N— | H | H | H | H | CF₃O | H | H | H |
| Compound 514 | >C═CH₂ | H | >C═CH₂ | >N— | H | H | H | H | H | H | H | H |
| Compound 515 | >C═CH₂ | H | >C═CH₂ | >N— | H | H | H | H | CH₃O | CH₃O | H | H |
| Compound 516 | >C═CH₂ | H | >C═CH₂ | >N— | H | H | H | H | CH₃O | H | H | H |
| Compound 517 | >C═CH₂ | H | >C═CH₂ | >N— | H | H | H | H | CF₃O | CF₃O | H | H |
| Compound 518 | >C═CH₂ | H | >C═CH₂ | >N— | H | H | H | H | CF₃O | H | H | H |
| Compound 519 | >C═NH | H | >C═NH | >N— | H | H | H | H | H | H | H | H |
| Compound 510 | >C═NH | H | >C═NH | >N— | H | H | H | H | CH₃O | CH₃O | H | H |
| Compound 521 | >C═NH | H | >C═NH | >N— | H | H | H | H | CH₃O | H | H | H |
| Compound 522 | >C═NH | H | >C═NH | >N— | H | H | H | H | CF₃O | CF₃O | H | H |
| Compound 523 | >C═NH | H | >C═NH | >N— | H | H | H | H | CF₃O | H | H | H |
| Compound 524 | >NCH₃ | H | >NCH₃ | >N— | H | H | H | H | H | H | H | H |
| Compound 525 | >NCH₃ | H | >NCH₃ | >N— | H | H | H | H | CH₃O | CH₃O | H | H |
| Compound 526 | >NCH₃ | H | >NCH₃ | >N— | H | H | H | H | CH₃O | H | H | H |
| Compound 527 | >NCH₃ | H | >NCH₃ | >N— | H | H | H | H | CF₃O | CF₃O | H | H |
| Compound 528 | >NCH₃ | H | >NCH₃ | >N— | H | H | H | H | CF₃O | H | H | H |
| Compound 529 | —O— | H | —O— | >B— | H | H | H | H | H | H | H | H |
| Compound 530 | —O— | H | —O— | >B— | H | H | H | H | CH₃O | CH₃O | H | H |
| Compound 531 | —O— | H | —O— | >B— | H | H | H | H | CH₃O | H | H | H |
| Compound 532 | —O— | H | —O— | >B— | H | H | H | H | CF₃O | CF₃O | H | H |
| Compound 533 | —O— | H | —O— | >B— | H | H | H | H | CF₃O | H | H | H |
| Compound 534 | —O— | H | —O— | >P(═O)— | H | H | H | H | H | H | H | H |
| Compound 535 | —O— | H | —O— | >P(═O)— | H | H | H | H | CH₃O | CH₃O | H | H |
| Compound 536 | —O— | H | —O— | >P(═O)— | H | H | H | H | CH₃O | H | H | H |
| Compound 537 | —O— | H | —O— | >P(═O)— | H | H | H | H | CF₃O | CF₃O | H | H |
| Compound 538 | —O— | H | —O— | >P(═O)— | H | H | H | H | CF₃O | H | H | H |
| Compound 539 | —O— | —O— | H | >N— | H | H | CF₃ | H | H | H | H | H |
| Compound 540 | —O— | —O— | H | >N— | H | H | H | CF₃ | H | H | H | H |
| Compound 541 | —O— | —O— | H | >N— | H | H | H | H | CF₃ | H | H | H |
| Compound 542 | —O— | —O— | H | >N— | H | H | H | H | H | CF₃ | H | H |
| Compound 543 | —O— | —O— | H | >N— | H | H | H | H | H | H | CF₃ | H |
| Compound 544 | —O— | —O— | H | >N— | H | H | H | H | H | H | H | CF₃ |
| Compound 545 | —O— | —O— | H | >N— | H | H | CF₃ | CF₃ | H | H | H | H |
| Compound 546 | —O— | —O— | H | >N— | H | H | CF₃ | H | CF₃ | H | H | H |
| Compound 547 | —O— | —O— | H | >N— | H | H | CF₃ | H | H | CF₃ | H | H |
| Compound 548 | —O— | —O— | H | >N— | H | H | CF₃ | H | H | H | CF₃ | H |
| Compound 549 | —O— | —O— | H | >N— | H | H | CF₃ | H | H | H | H | CF₃ |
| Compound 550 | —O— | —O— | H | >N— | H | H | H | CF₃ | CF₃ | H | H | H |
| Compound 551 | —O— | —O— | H | >N— | H | H | H | CF₃ | H | CF₃ | H | H |
| Compound 552 | —O— | —O— | H | >N— | H | H | H | CF₃ | H | H | CF₃ | H |
| Compound 553 | —O— | —O— | H | >N— | H | H | H | CF₃ | H | H | H | CF₃ |
| Compound 554 | —O— | —O— | H | >N— | H | H | H | H | CF₃ | CF₃ | H | H |
| Compound 555 | —O— | —O— | H | >N— | H | H | H | H | CF₃ | H | CF₃ | H |
| Compound 556 | —O— | —O— | H | >N— | H | H | H | H | CF₃ | H | H | CF₃ |
| Compound 557 | —O— | —O— | H | >N— | H | H | H | H | H | CF₃ | CF₃ | H |
| Compound 558 | —O— | —O— | H | >N— | H | H | H | H | H | CF₃ | H | CF₃ |
| Compound 559 | —O— | —O— | H | >N— | H | H | H | H | H | H | CF₃ | CF₃ |
| Compound 560 | —O— | H | —O— | >N— | H | H | CF₃ | H | H | H | H | H |
| Compound 561 | —O— | H | —O— | >N— | H | H | H | CF₃ | H | H | H | H |
| Compound 562 | —O— | H | —O— | >N— | H | H | H | H | CF₃ | H | H | H |
| Compound 563 | —O— | H | —O— | >N— | H | H | H | H | H | CF₃ | H | H |
| Compound 564 | —O— | H | —O— | >N— | H | H | H | H | H | H | CF₃ | H |
| Compound 565 | —O— | H | —O— | >N— | H | H | H | H | H | H | H | CF₃ |
| Compound 566 | —O— | H | —O— | >N— | H | H | CF₃ | CF₃ | H | H | H | H |
| Compound 567 | —O— | H | —O— | >N— | H | H | CF₃ | H | CF₃ | H | H | H |
| Compound 568 | —O— | H | —O— | >N— | H | H | CF₃ | H | H | CF₃ | H | H |
| Compound 569 | —O— | H | —O— | >N— | H | H | CF₃ | H | H | H | CF₃ | H |
| Compound 570 | —O— | H | —O— | >N— | H | H | CF₃ | H | H | H | H | CF₃ |
| Compound 571 | —O— | H | —O— | >N— | H | H | H | CF₃ | CF₃ | H | H | H |
| Compound 572 | —O— | H | —O— | >N— | H | H | H | CF₃ | H | CF₃ | H | H |
| Compound 573 | —O— | H | —O— | >N— | H | H | H | CF₃ | H | H | CF₃ | H |
| Compound 574 | —O— | H | —O— | >N— | H | H | H | CF₃ | H | H | H | CF₃ |
| Compound 575 | —O— | H | —O— | >N— | H | H | H | H | CF₃ | CF₃ | H | H |
| Compound 576 | —O— | H | —O— | >N— | H | H | H | H | CF₃ | H | CF₃ | H |
| Compound 577 | —O— | H | —O— | >N— | H | H | H | H | CF₃ | H | H | CF₃ |
| Compound 578 | —O— | H | —O— | >N— | H | H | H | H | H | CF₃ | CF₃ | H |
| Compound 579 | —O— | H | —O— | >N— | H | H | H | H | H | CF₃ | H | CF₃ |
| Compound 580 | —O— | H | —O— | >N— | H | H | H | H | H | H | CF₃ | CF₃ |

TABLE 6

| | General Formula [5] | | | | | | |
|---|---|---|---|---|---|---|---|
| | | | | | | | R³, R⁶ |
| Compound | | L¹ and L², | L³ and L³, | | | | and |
| No. | X¹, X⁴ | L⁵ and L⁶ | L⁷ and L⁹ | Y¹, Y² | R¹, R⁴ | R², R² | R¹³, R¹⁴ |
| Compound 601 | —O— | —O— | H | >N— | H | H | —CH═CH—CH═CH— |
| Compound 602 | —O— | —O— | H | >N— | H | H | —CH═CH—CH═CH— |
| Compound 603 | —O— | —O— | H | >N— | H | H | |
| Compound 604 | —O— | —O— | H | >N— | H | H | |
| Compound 605 | —O— | —O— | H | >N— | H | H | —CH═CH—CH═CH— |
| Compound 606 | —S— | —S— | H | >N— | H | H | —CH═CH—CH═CH— |
| Compound 607 | —S— | —S— | H | >N— | H | H | —CH═CH—CH═CH— |
| Compound 608 | —S— | —S— | H | >N— | H | H | |
| Compound 609 | —S— | —S— | H | >N— | H | H | |
| Compound 610 | —S— | —S— | H | >N— | H | H | —CH═CH—CH═CH— |
| Compound 611 | —SO₂— | —SO₂— | H | >N— | H | H | —CH═CH—CH═CH— |
| Compound 612 | —SO₂— | —SO₂— | H | >N— | H | H | —CH═CH—CH═CH— |
| Compound 613 | —SO₂— | —SO₂— | H | >N— | H | H | |
| Compound 614 | —SO₂— | —SO₂— | H | >N— | H | H | |
| Compound 615 | —SO₂— | —SO₂— | H | >N— | H | H | —CH═CH—CH═CH— |
| Compound 616 | —CH₂— | —CH₂— | H | >N— | H | H | —CH═CH—CH═CH— |
| Compound 617 | —CH₂— | —CH₂— | H | >N— | H | H | —CH═CH—CH═CH— |
| Compound 618 | —CH₂— | —CH₂— | H | >N— | H | H | |
| Compound 619 | —CH₂— | —CH₂— | H | >N— | H | H | |
| Compound 620 | —CH₂— | —CH₂— | H | >N— | H | H | —CH═CH—CH═CH— |
| Compound 621 | >C═O | >C═O | H | >N— | H | H | —CH═CH—CH═CH— |
| Compound 622 | >C═O | >C═O | H | >N— | H | H | —CH═CH—CH═CH— |
| Compound 623 | >C═O | >C═O | H | >N— | H | H | |
| Compound 624 | >C═O | >C═O | H | >N— | H | H | |
| Compound 625 | >C═O | >C═O | H | >N— | H | H | —CH═CH—CH═CH— |
| Compound 626 | >C═CH₂ | >C═CH₂ | H | >N— | H | H | —CH═CH—CH═CH— |
| Compound 627 | >C═CH₂ | >C═CH₂ | H | >N— | H | H | —CH═CH—CH═CH— |
| Compound 628 | >C═CH₂ | >C═CH₂ | H | >N— | H | H | |
| Compound 629 | >C═CH₂ | >C═CH₂ | H | >N— | H | H | |
| Compound 630 | >C═CH₂ | >C═CH₂ | H | >N— | H | H | —CH═CH—CH═CH— |
| Compound 631 | >C═NH | >C═NH | H | >N— | H | H | —CH═CH—CH═CH— |
| Compound 632 | >C═NH | >C═NH | H | >N— | H | H | —CH═CH—CH═CH— |
| Compound 633 | >C═NH | >C═NH | H | >N— | H | H | |
| Compound 634 | >C═NH | >C═NH | H | >N— | H | H | |
| Compound 635 | >C═NH | >C═NH | H | >N— | H | H | —CH═CH—CH═CH— |
| Compound 636 | >NCH₃ | >NCH₃ | H | >N— | H | H | —CH═CH—CH═CH— |
| Compound 637 | >NCH₃ | >NCH₃ | H | >N— | H | H | —CH═CH—CH═CH— |
| Compound 638 | >NCH₃ | >NCH₃ | H | >N— | H | H | |
| Compound 639 | >NCH₃ | >NCH₃ | H | >N— | H | H | |
| Compound 640 | >NCH₃ | >NCH₃ | H | >N— | H | H | —CH═CH—CH═CH— |
| Compound 641 | —O— | —O— | H | >B— | H | H | —CH═CH—CH═CH— |
| Compound 642 | —O— | —O— | H | >B— | H | H | —CH═CH—CH═CH— |
| Compound 643 | —O— | —O— | H | >B— | H | H | |
| Compound 644 | —O— | —O— | H | >B— | H | H | |
| Compound 645 | —O— | —O— | H | >B— | H | H | —CH═CH—CH═CH— |
| Compound 646 | —O— | —O— | H | >P(═O)— | H | H | —CH═CH—CH═CH— |
| Compound 647 | —O— | —O— | H | >P(═O)— | H | H | —CH═CH—CH═CH— |
| Compound 648 | —O— | —O— | H | >P(═O)— | H | H | |
| Compound 649 | —O— | —O— | H | >P(═O)— | H | H | |
| Compound 650 | —O— | —O— | H | >P(═O)— | H | H | —CH═CH—CH═CH— |
| Compound 651 | —O— | H | —O— | >N— | H | H | —CH═CH—CH═CH— |
| Compound 652 | —O— | H | —O— | >N— | H | H | —CH═CH—CH═CH— |
| Compound 653 | —O— | H | —O— | >N— | H | H | |
| Compound 654 | —O— | H | —O— | >N— | H | H | |
| Compound 655 | —O— | H | —O— | >N— | H | H | —CH═CH—CH═CH— |
| Compound 656 | —S— | H | —S— | >N— | H | H | —CH═CH—CH═CH— |
| Compound 657 | —S— | H | —S— | >N— | H | H | —CH═CH—CH═CH— |
| Compound 658 | —S— | H | —S— | >N— | H | H | |
| Compound 659 | —S— | H | —S— | >N— | H | H | |
| Compound 660 | —S— | H | —S— | >N— | H | H | —CH═CH—CH═CH— |
| Compound 661 | —SO₂— | H | —SO₂— | >N— | H | H | —CH═CH—CH═CH— |
| Compound 662 | —SO₂— | H | —SO₂— | >N— | H | H | —CH═CH—CH═CH— |
| Compound 663 | —SO₂— | H | —SO₂— | >N— | H | H | |
| Compound 664 | —SO₂— | H | —SO₂— | >N— | H | H | |
| Compound 665 | —SO₂— | H | —SO₂— | >N— | H | H | —CH═CH—CH═CH— |
| Compound 666 | —CH₂— | H | —CH₂— | >N— | H | H | —CH═CH—CH═CH— |
| Compound 667 | —CH₂— | H | —CH₂— | >N— | H | H | —CH═CH—CH═CH— |
| Compound 668 | —CH₂— | H | —CH₂— | >N— | H | H | |
| Compound 669 | —CH₂— | H | —CH₂— | >N— | H | H | |
| Compound 670 | —CH₂— | H | —CH₂— | >N— | H | H | —CH═CH—CH═CH— |
| Compound 671 | >C═O— | H | >C═O— | >N— | H | H | —CH═CH—CH═CH— |
| Compound 672 | >C═O— | H | >C═O— | >N— | H | H | —CH═CH—CH═CH— |
| Compound 673 | >C═O— | H | >C═O— | >N— | H | H | |
| Compound 674 | >C═O— | H | >C═O— | >N— | H | H | |
| Compound 675 | >C═O— | H | >C═O— | >N— | H | H | —CH═CH—CH═CH— |
| Compound 676 | >C═CH₂ | H | >C═CH₂ | >N— | H | H | —CH═CH—CH═CH— |
| Compound 677 | >C═CH₂ | H | >C═CH₂ | >N— | H | H | —CH═CH—CH═CH— |
| Compound 678 | >C═CH₂ | H | >C═CH₂ | >N— | H | H | |
| Compound 679 | >C═CH₂ | H | >C═CH₂ | >N— | H | H | |
| Compound 680 | >C═CH₂ | H | >C═CH₂ | >N— | H | H | —CH═CH—CH═CH— |
| Compound 681 | >C═NH | H | >C═NH | >N— | H | H | —CH═CH—CH═CH— |
| Compound 682 | >C═NH | H | >C═NH | >N— | H | H | —CH═CH—CH═CH— |
| Compound 683 | >C═NH | H | >C═NH | >N— | H | H | |
| Compound 684 | >C═NH | H | >C═NH | >N— | H | H | |
| Compound 685 | >C═NH | H | >C═NH | >N— | H | H | —CH═CH—CH═CH— |
| Compound 686 | >NCH₃ | H | >NCH₃ | >N— | H | H | —CH═CH—CH═CH— |
| Compound 687 | >NCH₃ | H | >NCH₃ | >N— | H | H | —CH═CH—CH═CH— |
| Compound 688 | >NCH₃ | H | >NCH₃ | >N— | H | H | |
| Compound 689 | >NCH₃ | H | >NCH₃ | >N— | H | H | |
| Compound 690 | >NCH₃ | H | >NCH₃ | >N— | H | H | —CH═CH—CH═CH— |
| Compound 691 | —O— | H | —O— | >B— | H | H | —CH═CH—CH═CH— |
| Compound 692 | —O— | H | —O— | >B— | H | H | —CH═CH—CH═CH— |
| Compound 693 | —O— | H | —O— | >B— | H | H | |
| Compound 694 | —O— | H | —O— | >B— | H | H | |
| Compound 695 | —O— | H | —O— | >B— | H | H | —CH═CH—CH═CH— |
| Compound 696 | —O— | H | —O— | >P(═O)— | H | H | —CH═CH—CH═CH— |
| Compound 697 | —O— | H | —O— | >P(═O)— | H | H | —CH═CH—CH═CH— |
| Compound 698 | —O— | H | —O— | >P(═O)— | H | H | |
| Compound 699 | —O— | H | —O— | >P(═O)— | H | H | |
| Compound 700 | —O— | H | —O— | >P(═O)— | H | H | —CH═CH—CH═CH— |

| | General Formula [5] | | | | | |
|---|---|---|---|---|---|---|
| | R¹¹, R¹² | | R⁶, R⁷ | R¹⁶, R¹⁷ | | |
| Compound | and | R⁷, R¹⁰, | and | and | R⁹, R¹² | R⁷, R¹² |
| No. | R¹⁹, R²⁰ | R¹⁵, R¹⁶ | R¹⁴, R¹⁵ | R¹⁸, R¹⁹ | R¹⁹, R²⁰ | R¹⁹, R²⁰ |
| Compound 601 | H | H | | | | |
| Compound 602 | —CH═CH—CH═CH— | H | | | | |
| Compound 603 | | | —CH═CH—CH═CH— | H | H | |
| Compound 604 | | | —CH═CH—CH═CH— | —CH═CH—CH═CH— | H | |
| Compound 605 | | | | —CH═CH—CH═CH— | | H |
| Compound 606 | H | H | | | | |
| Compound 607 | —CH═CH—CH═CH— | H | | | | |
| Compound 608 | | | —CH═CH—CH═CH— | H | H | |
| Compound 609 | | | —CH═CH—CH═CH— | —CH═CH—CH═CH— | H | |
| Compound 610 | | | | —CH═CH—CH═CH— | | H |
| Compound 611 | H | H | | | | |
| Compound 612 | —CH═CH—CH═CH— | H | | | | |
| Compound 613 | | | —CH═CH—CH═CH— | H | H | |
| Compound 614 | | | —CH═CH—CH═CH— | —CH═CH—CH═CH— | H | |
| Compound 615 | | | | —CH═CH—CH═CH— | | H |
| Compound 616 | H | H | | | | |
| Compound 617 | —CH═CH—CH═CH— | H | | | | |
| Compound 618 | | | —CH═CH—CH═CH— | H | H | |
| Compound 619 | | | —CH═CH—CH═CH— | —CH═CH—CH═CH— | H | |
| Compound 620 | | | | —CH═CH—CH═CH— | | H |
| Compound 621 | H | H | | | | |
| Compound 622 | —CH═CH—CH═CH— | H | | | | |
| Compound 623 | | | —CH═CH—CH═CH— | H | H | |
| Compound 624 | | | —CH═CH—CH═CH— | —CH═CH—CH═CH— | H | |
| Compound 625 | | | | —CH═CH—CH═CH— | | H |
| Compound 626 | H | H | | | | |
| Compound 627 | —CH═CH—CH═CH— | H | | | | |
| Compound 628 | | | —CH═CH—CH═CH— | H | H | |
| Compound 629 | | | —CH═CH—CH═CH— | —CH═CH—CH═CH— | H | |
| Compound 630 | | | | —CH═CH—CH═CH— | | H |
| Compound 631 | H | H | | | | |
| Compound 632 | —CH═CH—CH═CH— | H | | | | |
| Compound 633 | | | —CH═CH—CH═CH— | H | H | |
| Compound 634 | | | —CH═CH—CH═CH— | —CH═CH—CH═CH— | H | |
| Compound 635 | | | | —CH═CH—CH═CH— | | H |
| Compound 636 | H | H | | | | |
| Compound 637 | —CH═CH—CH═CH— | H | | | | |
| Compound 638 | | | —CH═CH—CH═CH— | H | H | |
| Compound 639 | | | —CH═CH—CH═CH— | —CH═CH—CH═CH— | H | |
| Compound 640 | | | | —CH═CH—CH═CH— | | H |
| Compound 641 | H | H | | | | |
| Compound 642 | —CH═CH—CH═CH— | H | | | | |
| Compound 643 | | | —CH═CH—CH═CH— | H | H | |
| Compound 644 | | | —CH═CH—CH═CH— | —CH═CH—CH═CH— | H | |
| Compound 645 | | | | —CH═CH—CH═CH— | | H |
| Compound 646 | H | H | | | | |
| Compound 647 | —CH═CH—CH═CH— | H | | | | |
| Compound 648 | | | —CH═CH—CH═CH— | H | H | |
| Compound 649 | | | —CH═CH—CH═CH— | —CH═CH—CH═CH— | H | |
| Compound 650 | | | | —CH═CH—CH═CH— | | H |
| Compound 651 | H | H | | | | |
| Compound 652 | —CH═CH—CH═CH— | H | | | | |
| Compound 653 | | | —CH═CH—CH═CH— | H | H | |
| Compound 654 | | | —CH═CH—CH═CH— | —CH═CH—CH═CH— | H | |
| Compound 655 | | | | —CH═CH—CH═CH— | | H |
| Compound 656 | H | H | | | | |
| Compound 657 | —CH═CH—CH═CH— | H | | | | |
| Compound 658 | | | —CH═CH—CH═CH— | H | H | |
| Compound 659 | | | —CH═CH—CH═CH— | —CH═CH—CH═CH— | H | |
| Compound 660 | | | | —CH═CH—CH═CH— | | H |
| Compound 661 | H | H | | | | |
| Compound 662 | —CH═CH—CH═CH— | H | | | | |
| Compound 663 | | | —CH═CH—CH═CH— | H | H | |
| Compound 664 | | | —CH═CH—CH═CH— | —CH═CH—CH═CH— | H | |
| Compound 665 | | | | —CH═CH—CH═CH— | | H |
| Compound 666 | H | H | | | | |
| Compound 667 | —CH═CH—CH═CH— | H | | | | |
| Compound 668 | | | —CH═CH—CH═CH— | H | H | |
| Compound 669 | | | —CH═CH—CH═CH— | —CH═CH—CH═CH— | H | |
| Compound 670 | | | | —CH═CH—CH═CH— | | H |
| Compound 671 | H | H | | | | |
| Compound 672 | —CH═CH—CH═CH— | H | | | | |
| Compound 673 | | | —CH═CH—CH═CH— | H | H | |
| Compound 674 | | | —CH═CH—CH═CH— | —CH═CH—CH═CH— | H | |
| Compound 675 | | | | —CH═CH—CH═CH— | | H |
| Compound 676 | H | H | | | | |
| Compound 677 | —CH═CH—CH═CH— | H | | | | |
| Compound 678 | | | —CH═CH—CH═CH— | H | H | |
| Compound 679 | | | —CH═CH—CH═CH— | —CH═CH—CH═CH— | H | |
| Compound 680 | | | | —CH═CH—CH═CH— | | H |
| Compound 681 | H | H | | | | |
| Compound 682 | —CH═CH—CH═CH— | H | | | | |
| Compound 683 | | | —CH═CH—CH═CH— | H | H | |
| Compound 684 | | | —CH═CH—CH═CH— | —CH═CH—CH═CH— | H | |
| Compound 685 | | | | —CH═CH—CH═CH— | | H |
| Compound 686 | H | H | | | | |
| Compound 687 | —CH═CH—CH═CH— | H | | | | |
| Compound 688 | | | —CH═CH—CH═CH— | H | H | |
| Compound 689 | | | —CH═CH—CH═CH— | —CH═CH—CH═CH— | H | |
| Compound 690 | | | | —CH═CH—CH═CH— | | H |
| Compound 691 | H | H | | | | |
| Compound 692 | —CH═CH—CH═CH— | H | | | | |
| Compound 693 | | | —CH═CH—CH═CH— | H | H | |
| Compound 694 | | | —CH═CH—CH═CH— | —CH═CH—CH═CH— | H | |
| Compound 695 | | | | —CH═CH—CH═CH— | | H |
| Compound 696 | H | H | | | | |
| Compound 697 | —CH═CH—CH═CH— | H | | | | |
| Compound 698 | | | —CH═CH—CH═CH— | H | H | |
| Compound 699 | | | —CH═CH—CH═CH— | —CH═CH—CH═CH— | H | |
| Compound 700 | | | | —CH═CH—CH═CH— | | H |

TABLE 7

| | General Formula [6] | | | | | | | | | | | |
|---|---|---|---|---|---|---|---|---|---|---|---|---|
| Compound | | L¹ and L² | L³ and L⁴ | | | | | | | | | |
| No. | X³, X⁴ | L⁵ and L⁶ | L⁷ and L⁸ | Y³, Y² | R², R⁴ | R², R³ | R³, R¹³ | R⁸, R¹⁴ | R⁷, R¹³ | R¹⁰, R¹⁸ | R¹¹, R¹⁵ | R¹², R²⁰ |
| Compound 701 | —O— | —O— | H | >N— | H | H | H | H | H | H | H | H |
| Compound 702 | —O— | —O— | H | >N— | H | H | H | H | CH₃O | CH₃O | H | H |
| Compound 703 | —O— | —O— | H | >N— | H | H | H | H | CH₃O | H | H | H |
| Compound 704 | —O— | —O— | H | >N— | H | H | H | H | CH₃ | H | H | H |
| Compound 705 | —O— | —O— | H | >N— | H | H | H | H | C₆H₅O | C₆H₅O | H | H |
| Compound 706 | —O— | —O— | H | >N— | H | H | H | H | C₆H₅ | C₆H₅ | H | H |
| Compound 707 | —O— | —O— | H | >N— | H | H | H | H | CF₃O | CF₃O | H | H |
| Compound 708 | —O— | —O— | H | >N— | H | H | H | H | CF₃O | H | H | H |
| Compound 709 | —O— | —O— | H | >N— | H | H | H | H | H | H | H | H |
| Compound 710 | —O— | —O— | H | >N— | H | H | CH₃O | CH₃O | H | H | H | H |
| Compound 711 | —O— | —O— | H | >N— | H | H | CH₃O | H | H | H | H | H |
| Compound 712 | —O— | —O— | H | >N— | H | H | CH₃ | H | H | H | H | H |
| Compound 713 | —O— | —O— | H | >N— | H | H | C₆H₅O | C₆H₅O | H | H | H | H |
| Compound 714 | —O— | —O— | H | >N— | H | H | C₆H₅ | C₆H₅ | H | H | H | H |
| Compound 715 | —O— | —O— | H | >N— | H | H | CF₃O | CF₃O | H | H | H | H |
| Compound 716 | —O— | —O— | H | >N— | H | H | CF₃O | H | H | H | H | H |
| Compound 717 | —O— | —O— | H | >N— | H | H | H | H | H | H | H | H |
| Compound 718 | —O— | —O— | H | >N— | H | H | H | H | H | H | CH₃O | CH₃O |
| Compound 719 | —O— | —O— | H | >N— | H | H | H | H | H | H | CH₃O | H |
| Compound 720 | —O— | —O— | H | >N— | H | H | H | H | H | H | CH₃ | H |
| Compound 721 | —O— | —O— | H | >N— | H | H | H | H | H | H | C₆H₅O | C₆H₅O |
| Compound 722 | —O— | —O— | H | >N— | H | H | H | H | H | H | C₆H₅ | C₆H₅ |
| Compound 723 | —O— | —O— | H | >N— | H | H | H | H | H | H | CF₃O | H |
| Compound 724 | —O— | —O— | H | >N— | H | H | H | H | H | H | CF₃O | H |
| Compound 725 | —S— | —S— | H | >N— | H | H | H | H | H | H | H | H |
| Compound 726 | —S— | —S— | H | >N— | H | H | H | H | CH₃O | CH₃O | H | H |
| Compound 727 | —S— | —S— | H | >N— | H | H | H | H | CH₃O | H | H | H |
| Compound 728 | —S— | —S— | H | >N— | H | H | H | H | CF₃O | CF₃O | H | H |
| Compound 729 | —S— | —S— | H | >N— | H | H | H | H | CF₃O | H | H | H |
| Compound 730 | —SO₂— | —SO₂— | H | >N— | H | H | H | H | H | H | H | H |
| Compound 731 | —SO₂— | —SO₂— | H | >N— | H | H | H | H | CH₃O | CH₃O | H | H |
| Compound 732 | —SO₂— | —SO₂— | H | >N— | H | H | H | H | CH₃O | H | H | H |
| Compound 733 | —SO₂— | —SO₂— | H | >N— | H | H | H | H | CF₃O | CF₃O | H | H |
| Compound 734 | —SO₂— | —SO₂— | H | >N— | H | H | H | H | CF₃O | H | H | H |
| Compound 735 | —CH₂— | —CH₂— | H | >N— | H | H | H | H | H | H | H | H |
| Compound 736 | —CH₂— | —CH₂— | H | >N— | H | H | H | H | CH₃O | CH₃O | H | H |
| Compound 737 | —CH₂— | —CH₂— | H | >N— | H | H | H | H | CH₃O | H | H | H |
| Compound 738 | —CH₂— | —CH₂— | H | >N— | H | H | H | H | CF₃O | CF₃O | H | H |
| Compound 739 | —CH₂— | —CH₂— | H | >N— | H | H | H | H | CF₃O | H | H | H |
| Compound 740 | >C═O | >C═O | H | >N— | H | H | H | H | H | H | H | H |
| Compound 741 | >C═O | >C═O | H | >N— | H | H | H | H | CH₃O | CH₃O | H | H |
| Compound 742 | >C═O | >C═O | H | >N— | H | H | H | H | CH₃O | H | H | H |
| Compound 743 | >C═O | >C═O | H | >N— | H | H | H | H | CF₃O | CF₃O | H | H |
| Compound 744 | >C═O | >C═O | H | >N— | H | H | H | H | CF₃O | H | H | H |
| Compound 745 | >C═CH₂ | >C═CH₂ | H | >N— | H | H | H | H | H | H | H | H |
| Compound 746 | >C═CH₂ | >C═CH₂ | H | >N— | H | H | H | H | CH₃O | CH₃O | H | H |
| Compound 747 | >C═CH₂ | >C═CH₂ | H | >N— | H | H | H | H | CH₃O | H | H | H |
| Compound 748 | >C═CH₂ | >C═CH₂ | H | >N— | H | H | H | H | CF₃O | CF₃O | H | H |
| Compound 749 | >C═CH₂ | >C═CH₂ | H | >N— | H | H | H | H | CF₃O | H | H | H |
| Compound 750 | >C═NH | >C═NH | H | >N— | H | H | H | H | H | H | H | H |
| Compound 751 | >C═NH | >C═NH | H | >N— | H | H | H | H | CH₃O | CH₃O | H | H |
| Compound 752 | >C═NH | >C═NH | H | >N— | H | H | H | H | CH₃O | H | H | H |
| Compound 753 | >C═NH | >C═NH | H | >N— | H | H | H | H | CF₃O | CF₃O | H | H |
| Compound 754 | >C═NH | >C═NH | H | >N— | H | H | H | H | CF₃O | H | H | H |
| Compound 755 | >NCH₃ | >NCH₃ | H | >N— | H | H | H | H | H | H | H | H |
| Compound 756 | >NCH₃ | >NCH₃ | H | >N— | H | H | H | H | CH₃O | CH₃O | H | H |
| Compound 757 | >NCH₃ | >NCH₃ | H | >N— | H | H | H | H | CH₃O | H | H | H |
| Compound 758 | >NCH₃ | >NCH₃ | H | >N— | H | H | H | H | CF₃O | CF₃O | H | H |
| Compound 759 | >NCH₃ | >NCH₃ | H | >N— | H | H | H | H | CF₃O | H | H | H |
| Compound 760 | —O— | —O— | H | >B— | H | H | H | H | H | H | H | H |
| Compound 761 | —O— | —O— | H | >B— | H | H | H | H | CH₃O | CH₃O | H | H |
| Compound 762 | —O— | —O— | H | >B— | H | H | H | H | CH₃O | H | H | H |
| Compound 763 | —O— | —O— | H | >B— | H | H | H | H | CF₃O | CF₃O | H | H |
| Compound 764 | —O— | —O— | H | >B— | H | H | H | H | CF₃O | H | H | H |
| Compound 765 | —O— | —O— | H | >P(═O)— | H | H | H | H | H | H | H | H |
| Compound 766 | —O— | —O— | H | >P(═O)— | H | H | H | H | CH₃O | CH₃O | H | H |
| Compound 767 | —O— | —O— | H | >P(═O)— | H | H | H | H | CH₃O | H | H | H |
| Compound 768 | —O— | —O— | H | >P(═O)— | H | H | H | H | CF₃O | CF₃O | H | H |
| Compound 769 | —O— | —O— | H | >P(═O)— | H | H | H | H | CF₃O | H | H | H |
| Compound 770 | —O— | H | —O— | >N— | H | H | H | H | H | H | H | H |
| Compound 771 | —O— | H | —O— | >N— | H | H | H | H | CH₃O | CH₃O | H | H |
| Compound 772 | —O— | H | —O— | >N— | H | H | H | H | CH₃O | H | H | H |
| Compound 773 | —O— | H | —O— | >N— | H | H | H | H | CH₃ | H | H | H |
| Compound 774 | —O— | H | —O— | >N— | H | H | H | H | C₆H₅O | C₆H₅O | H | H |
| Compound 775 | —O— | H | —O— | >N— | H | H | H | H | C₆H₅ | C₆H₅ | H | H |
| Compound 776 | —O— | H | —O— | >N— | H | H | H | H | CF₃O | CF₃O | H | H |
| Compound 777 | —O— | H | —O— | >N— | H | H | H | H | CF₃O | H | H | H |
| Compound 778 | —O— | H | —O— | >N— | H | H | H | H | H | H | H | H |
| Compound 779 | —O— | H | —O— | >N— | H | H | CH₃O | CH₃O | H | H | H | H |
| Compound 780 | —O— | H | —O— | >N— | H | H | CH₃O | H | H | H | H | H |
| Compound 781 | —O— | H | —O— | >N— | H | H | CH₃ | H | H | H | H | H |
| Compound 782 | —O— | H | —O— | >N— | H | H | C₆H₅O | C₆H₅O | H | H | H | H |
| Compound 783 | —O— | H | —O— | >N— | H | H | C₆H₅ | C₆H₅ | H | H | H | H |
| Compound 784 | —O— | H | —O— | >N— | H | H | CF₃O | CF₃O | H | H | H | H |
| Compound 785 | —O— | H | —O— | >N— | H | H | CF₃O | H | H | H | H | H |
| Compound 786 | —O— | H | —O— | >N— | H | H | H | H | H | H | H | H |
| Compound 787 | —O— | H | —O— | >N— | H | H | H | H | H | H | CH₃O | CH₃O |
| Compound 788 | —O— | H | —O— | >N— | H | H | H | H | H | H | CH₃O | H |
| Compound 789 | —O— | H | —O— | >N— | H | H | H | H | H | H | CH₃ | H |
| Compound 790 | —O— | H | —O— | >N— | H | H | H | H | H | H | C₆H₅O | C₆H₅O |
| Compound 791 | —O— | H | —O— | >N— | H | H | H | H | H | H | C₆H₅ | C₆H₅ |
| Compound 792 | —O— | H | —O— | >N— | H | H | H | H | H | H | CF₃O | CF₃O |
| Compound 793 | —O— | H | —O— | >N— | H | H | H | H | H | H | CF₃O | H |
| Compound 794 | —S— | H | —S— | >N— | H | H | H | H | H | H | H | H |
| Compound 795 | —S— | H | —S— | >N— | H | H | H | H | CH₃O | CH₃O | H | H |
| Compound 796 | —S— | H | —S— | >N— | H | H | H | H | CH₃O | H | H | H |
| Compound 797 | —S— | H | —S— | >N— | H | H | H | H | CF₃O | CF₃O | H | H |
| Compound 798 | —S— | H | —S— | >N— | H | H | H | H | CF₃O | H | H | H |
| Compound 799 | —SO₂— | H | —SO₂— | >N— | H | H | H | H | H | H | H | H |
| Compound 800 | —SO₂— | H | —SO₂— | >N— | H | H | H | H | CH₃O | CH₃O | H | H |
| Compound 801 | —SO₂— | H | —SO₂— | >N— | H | H | H | H | CH₃O | H | H | H |
| Compound 802 | —SO₂— | H | —SO₂— | >N— | H | H | H | H | CF₃O | CF₃O | H | H |
| Compound 803 | —SO₂— | H | —SO₂— | >N— | H | H | H | H | CF₃O | H | H | H |
| Compound 804 | —CH₂— | H | —CH₂— | >N— | H | H | H | H | H | H | H | H |
| Compound 805 | —CH₂— | H | —CH₂— | >N— | H | H | H | H | CH₃O | CH₃O | H | H |
| Compound 806 | —CH₂— | H | —CH₂— | >N— | H | H | H | H | CH₃O | H | H | H |
| Compound 807 | —CH₂— | H | —CH₂— | >N— | H | H | H | H | CF₃O | CF₃O | H | H |
| Compound 808 | —CH₂— | H | —CH₂— | >N— | H | H | H | H | CF₃O | H | H | H |
| Compound 809 | >C═O | H | >C═O | >N— | H | H | H | H | H | H | H | H |
| Compound 810 | >C═O | H | >C═O | >N— | H | H | H | H | CH₃O | CH₃O | H | H |
| Compound 811 | >C═O | H | >C═O | >N— | H | H | H | H | CH₃O | H | H | H |
| Compound 812 | >C═O | H | >C═O | >N— | H | H | H | H | CF₃O | CF₃O | H | H |
| Compound 813 | >C═O | H | >C═O | >N— | H | H | H | H | CF₃O | H | H | H |
| Compound 814 | >C═CH₂ | H | >C═CH₂ | >N— | H | H | H | H | H | H | H | H |
| Compound 815 | >C═CH₂ | H | >C═CH₂ | >N— | H | H | H | H | CH₃O | CH₃O | H | H |
| Compound 816 | >C═CH₂ | H | >C═CH₂ | >N— | H | H | H | H | CH₃O | H | H | H |
| Compound 817 | >C═CH₂ | H | >C═CH₂ | >N— | H | H | H | H | CF₃O | CF₃O | H | H |
| Compound 818 | >C═CH₂ | H | >C═CH₂ | >N— | H | H | H | H | CF₃O | H | H | H |
| Compound 819 | >C═NH | H | >C═NH | >N— | H | H | H | H | H | H | H | H |
| Compound 810 | >C═NH | H | >C═NH | >N— | H | H | H | H | CH₃O | CH₃O | H | H |
| Compound 821 | >C═NH | H | >C═NH | >N— | H | H | H | H | CH₃O | H | H | H |
| Compound 822 | >C═NH | H | >C═NH | >N— | H | H | H | H | CF₃O | CF₃O | H | H |
| Compound 823 | >C═NH | H | >C═NH | >N— | H | H | H | H | CF₃O | H | H | H |
| Compound 824 | >NCH₃ | H | >NCH₃ | >N— | H | H | H | H | H | H | H | H |
| Compound 825 | >NCH₃ | H | >NCH₃ | >N— | H | H | H | H | CH₃O | CH₃O | H | H |
| Compound 826 | >NCH₃ | H | >NCH₃ | >N— | H | H | H | H | CH₃O | H | H | H |
| Compound 827 | >NCH₃ | H | >NCH₃ | >N— | H | H | H | H | CF₃O | CF₃O | H | H |
| Compound 828 | >NCH₃ | H | >NCH₃ | >N— | H | H | H | H | CF₃O | H | H | H |
| Compound 829 | —O— | H | —O— | >B— | H | H | H | H | H | H | H | H |
| Compound 830 | —O— | H | —O— | >B— | H | H | H | H | CH₃O | CH₃O | H | H |
| Compound 831 | —O— | H | —O— | >B— | H | H | H | H | CH₃O | H | H | H |
| Compound 832 | —O— | H | —O— | >B— | H | H | H | H | CF₃O | CF₃O | H | H |
| Compound 833 | —O— | H | —O— | >B— | H | H | H | H | CF₃O | H | H | H |
| Compound 834 | —O— | H | —O— | >P(═O)— | H | H | H | H | H | H | H | H |
| Compound 835 | —O— | H | —O— | >P(═O)— | H | H | H | H | CH₃O | CH₃O | H | H |
| Compound 836 | —O— | H | —O— | >P(═O)— | H | H | H | H | CH₃O | H | H | H |
| Compound 837 | —O— | H | —O— | >P(═O)— | H | H | H | H | CF₃O | CF₃O | H | H |
| Compound 838 | —O— | H | —O— | >P(═O)— | H | H | H | H | CF₃O | H | H | H |
| Compound 839 | —O— | —O— | H | >N— | H | H | CF₃ | H | H | H | H | H |
| Compound 840 | —O— | —O— | H | >N— | H | H | H | CF₃ | H | H | H | H |
| Compound 841 | —O— | —O— | H | >N— | H | H | H | H | CF₃ | H | H | H |
| Compound 842 | —O— | —O— | H | >N— | H | H | H | H | H | CF₃ | H | H |
| Compound 843 | —O— | —O— | H | >N— | H | H | H | H | H | H | CF₃ | H |
| Compound 844 | —O— | —O— | H | >N— | H | H | H | H | H | H | H | CF₃ |
| Compound 845 | —O— | —O— | H | >N— | H | H | CF₃ | CF₃ | H | H | H | H |
| Compound 846 | —O— | —O— | H | >N— | H | H | CF₃ | H | CF₃ | H | H | H |
| Compound 847 | —O— | —O— | H | >N— | H | H | CF₃ | H | H | CF₃ | H | H |
| Compound 848 | —O— | —O— | H | >N— | H | H | CF₃ | H | H | H | CF₃ | H |
| Compound 849 | —O— | —O— | H | >N— | H | H | CF₃ | H | H | H | H | CF₃ |
| Compound 850 | —O— | —O— | H | >N— | H | H | H | CF₃ | CF₃ | H | H | H |
| Compound 851 | —O— | —O— | H | >N— | H | H | H | CF₃ | H | CF₃ | H | H |
| Compound 852 | —O— | —O— | H | >N— | H | H | H | CF₃ | H | H | CF₃ | H |
| Compound 853 | —O— | —O— | H | >N— | H | H | H | CF₃ | H | H | H | CF₃ |
| Compound 854 | —O— | —O— | H | >N— | H | H | H | H | CF₃ | CF₃ | H | H |
| Compound 855 | —O— | —O— | H | >N— | H | H | H | H | CF₃ | H | CF₃ | H |
| Compound 856 | —O— | —O— | H | >N— | H | H | H | H | CF₃ | H | H | CF₃ |
| Compound 857 | —O— | —O— | H | >N— | H | H | H | H | H | CF₃ | CF₃ | H |
| Compound 858 | —O— | —O— | H | >N— | H | H | H | H | H | CF₃ | H | CF₃ |
| Compound 859 | —O— | —O— | H | >N— | H | H | H | H | H | H | CF₃ | CF₃ |
| Compound 860 | —O— | H | —O— | >N— | H | H | CF₃ | H | H | H | H | H |
| Compound 861 | —O— | H | —O— | >N— | H | H | H | CF₃ | H | H | H | H |
| Compound 862 | —O— | H | —O— | >N— | H | H | H | H | CF₃ | H | H | H |
| Compound 863 | —O— | H | —O— | >N— | H | H | H | H | H | CF₃ | H | H |
| Compound 864 | —O— | H | —O— | >N— | H | H | H | H | H | H | CF₃ | H |
| Compound 865 | —O— | H | —O— | >N— | H | H | H | H | H | H | H | CF₃ |
| Compound 866 | —O— | H | —O— | >N— | H | H | CF₃ | CF₃ | H | H | H | H |
| Compound 867 | —O— | H | —O— | >N— | H | H | CF₃ | H | CF₃ | H | H | H |
| Compound 868 | —O— | H | —O— | >N— | H | H | CF₃ | H | H | CF₃ | H | H |
| Compound 869 | —O— | H | —O— | >N— | H | H | CF₃ | H | H | H | CF₃ | H |
| Compound 870 | —O— | H | —O— | >N— | H | H | CF₃ | H | H | H | H | CF₃ |
| Compound 871 | —O— | H | —O— | >N— | H | H | H | CF₃ | CF₃ | H | H | H |
| Compound 872 | —O— | H | —O— | >N— | H | H | H | CF₃ | H | CF₃ | H | H |
| Compound 873 | —O— | H | —O— | >N— | H | H | H | CF₃ | H | H | CF₃ | H |
| Compound 874 | —O— | H | —O— | >N— | H | H | H | CF₃ | H | H | H | CF₃ |
| Compound 875 | —O— | H | —O— | >N— | H | H | H | H | CF₃ | CF₃ | H | H |
| Compound 876 | —O— | H | —O— | >N— | H | H | H | H | CF₃ | H | CF₃ | H |
| Compound 877 | —O— | H | —O— | >N— | H | H | H | H | CF₃ | H | H | CF₃ |
| Compound 878 | —O— | H | —O— | >N— | H | H | H | H | H | CF₃ | CF₃ | H |
| Compound 879 | —O— | H | —O— | >N— | H | H | H | H | H | CF₃ | H | CF₃ |
| Compound 880 | —O— | H | —O— | >N— | H | H | H | H | H | H | CF₃ | CF₃ |

TABLE 8

| | General Formula [6] | | | | | | | |
|---|---|---|---|---|---|---|---|---|
| Compound | | L¹ and L², | L³ and L⁴, | | R¹, | R², | R⁸, R⁹ and | R¹¹, R¹² and |
| No. | X³, X⁴ | L⁵ and L⁶ | L⁷ and L⁸ | Y³, Y² | R² | R³ | R¹³, R¹⁴ | R¹³, R³⁰ |
| Compound 901 | —O— | —O— | H | >N— | H | H | —CH═CH—CH═CH— | H |
| Compound 902 | —O— | —O— | H | >N— | H | H | —CH═CH—CH═CH— | —CH═CH—CH═CH— |
| Compound 903 | —O— | —O— | H | >N— | H | H | | |
| Compound 904 | —O— | —O— | H | >N— | H | H | | |
| Compound 905 | —O— | —O— | H | >N— | H | H | —CH═CH—CH═CH— | |
| Compound 906 | —S— | —S— | H | >N— | H | H | —CH═CH—CH═CH— | H |
| Compound 907 | —S— | —S— | H | >N— | H | H | —CH═CH—CH═CH— | —CH═CH—CH═CH— |
| Compound 908 | —S— | —S— | H | >N— | H | H | | |
| Compound 909 | —S— | —S— | H | >N— | H | H | | |
| Compound 910 | —S— | —S— | H | >N— | H | H | —CH═CH—CH═CH— | |
| Compound 911 | —SO₂— | —SO₂— | H | >N— | H | H | —CH═CH—CH═CH— | H |
| Compound 912 | —SO₂— | —SO₂— | H | >N— | H | H | —CH═CH—CH═CH— | —CH═CH—CH═CH— |
| Compound 913 | —SO₂— | —SO₂— | H | >N— | H | H | | |
| Compound 914 | —SO₂— | —SO₂— | H | >N— | H | H | | |
| Compound 915 | —SO₂— | —SO₂— | H | >N— | H | H | —CH═CH—CH═CH— | |
| Compound 916 | —CH₂— | —CH₂— | H | >N— | H | H | —CH═CH—CH═CH— | H |
| Compound 917 | —CH₂— | —CH₂— | H | >N— | H | H | —CH═CH—CH═CH— | —CH═CH—CH═CH— |
| Compound 918 | —CH₂— | —CH₂— | H | >N— | H | H | | |
| Compound 919 | —CH₂— | —CH₂— | H | >N— | H | H | | |
| Compound 920 | —CH₂— | —CH₂— | H | >N— | H | H | —CH═CH—CH═CH— | |
| Compound 921 | >C═O | >C═O | H | >N— | H | H | —CH═CH—CH═CH— | H |
| Compound 922 | >C═O | >C═O | H | >N— | H | H | —CH═CH—CH═CH— | —CH═CH—CH═CH— |
| Compound 923 | >C═O | >C═O | H | >N— | H | H | | |
| Compound 924 | >C═O | >C═O | H | >N— | H | H | | |
| Compound 925 | >C═O | >C═O | H | >N— | H | H | —CH═CH—CH═CH— | |
| Compound 926 | >C═CH₂ | >C═CH₂ | H | >N— | H | H | —CH═CH—CH═CH— | H |
| Compound 927 | >C═CH₂ | >C═CH₂ | H | >N— | H | H | —CH═CH—CH═CH— | —CH═CH—CH═CH— |
| Compound 928 | >C═CH₂ | >C═CH₂ | H | >N— | H | H | | |
| Compound 929 | >C═CH₂ | >C═CH₂ | H | >N— | H | H | | |
| Compound 930 | >C═CH₂ | >C═CH₂ | H | >N— | H | H | —CH═CH—CH═CH— | |
| Compound 931 | >C═NH | >C═NH | H | >N— | H | H | —CH═CH—CH═CH— | H |
| Compound 932 | >C═NH | >C═NH | H | >N— | H | H | —CH═CH—CH═CH— | —CH═CH—CH═CH— |
| Compound 933 | >C═NH | >C═NH | H | >N— | H | H | | |
| Compound 934 | >C═NH | >C═NH | H | >N— | H | H | | |
| Compound 935 | >C═NH | >C═NH | H | >N— | H | H | —CH═CH—CH═CH— | |
| Compound 936 | >NCH₃ | >NCH₃ | H | >N— | H | H | —CH═CH—CH═CH— | H |
| Compound 937 | >NCH₃ | >NCH₃ | H | >N— | H | H | —CH═CH—CH═CH— | —CH═CH—CH═CH— |
| Compound 938 | >NCH₃ | >NCH₃ | H | >N— | H | H | | |
| Compound 939 | >NCH₃ | >NCH₃ | H | >N— | H | H | | |
| Compound 940 | >NCH₃ | >NCH₃ | H | >N— | H | H | —CH═CH—CH═CH— | |
| Compound 941 | —O— | —O— | H | >B— | H | H | —CH═CH—CH═CH— | H |
| Compound 942 | —O— | —O— | H | >B— | H | H | —CH═CH—CH═CH— | —CH═CH—CH═CH— |
| Compound 943 | —O— | —O— | H | >B— | H | H | | |
| Compound 944 | —O— | —O— | H | >B— | H | H | | |
| Compound 945 | —O— | —O— | H | >B— | H | H | —CH═CH—CH═CH— | |
| Compound 946 | —O— | —O— | H | >P(═O)— | H | H | —CH═CH—CH═CH— | H |
| Compound 947 | —O— | —O— | H | >P(═O)— | H | H | —CH═CH—CH═CH— | —CH═CH—CH═CH— |
| Compound 948 | —O— | —O— | H | >P(═O)— | H | H | | |
| Compound 949 | —O— | —O— | H | >P(═O)— | H | H | | |
| Compound 950 | —O— | —O— | H | >P(═O)— | H | H | —CH═CH—CH═CH— | |
| Compound 951 | —O— | H | —O— | >N— | H | H | —CH═CH—CH═CH— | H |
| Compound 952 | —O— | H | —O— | >N— | H | H | —CH═CH—CH═CH— | —CH═CH—CH═CH— |
| Compound 953 | —O— | H | —O— | >N— | H | H | | |
| Compound 954 | —O— | H | —O— | >N— | H | H | | |
| Compound 955 | —O— | H | —O— | >N— | H | H | —CH═CH—CH═CH— | |
| Compound 956 | —S— | H | —S— | >N— | H | H | —CH═CH—CH═CH— | H |
| Compound 957 | —S— | H | —S— | >N— | H | H | —CH═CH—CH═CH— | —CH═CH—CH═CH— |
| Compound 958 | —S— | H | —S— | >N— | H | H | | |
| Compound 959 | —S— | H | —S— | >N— | H | H | | |
| Compound 960 | —S— | H | —S— | >N— | H | H | —CH═CH—CH═CH— | |
| Compound 961 | —SO₂— | H | —SO₂— | >N— | H | H | —CH═CH—CH═CH— | H |
| Compound 962 | —SO₂— | H | —SO₂— | >N— | H | H | —CH═CH—CH═CH— | —CH═CH—CH═CH— |
| Compound 963 | —SO₂— | H | —SO₂— | >N— | H | H | | |
| Compound 964 | —SO₂— | H | —SO₂— | >N— | H | H | | |
| Compound 965 | —SO₂— | H | —SO₂— | >N— | H | H | —CH═CH—CH═CH— | |
| Compound 966 | —CH₂— | H | —CH₂— | >N— | H | H | —CH═CH—CH═CH— | H |
| Compound 967 | —CH₂— | H | —CH₂— | >N— | H | H | —CH═CH—CH═CH— | —CH═CH—CH═CH— |
| Compound 968 | —CH₂— | H | —CH₂— | >N— | H | H | | |
| Compound 969 | —CH₂— | H | —CH₂— | >N— | H | H | | |
| Compound 970 | —CH₂— | H | —CH₂— | >N— | H | H | —CH═CH—CH═CH— | |
| Compound 971 | >C═O | H | >C═O | >N— | H | H | —CH═CH—CH═CH— | H |
| Compound 972 | >C═O | H | >C═O | >N— | H | H | —CH═CH—CH═CH— | —CH═CH—CH═CH— |
| Compound 973 | >C═O | H | >C═O | >N— | H | H | | |
| Compound 974 | >C═O | H | >C═O | >N— | H | H | | |
| Compound 975 | >C═O | H | >C═O | >N— | H | H | —CH═CH—CH═CH— | |
| Compound 976 | >C═CH₂ | H | >C═CH₂ | >N— | H | H | —CH═CH—CH═CH— | H |
| Compound 977 | >C═CH₂ | H | >C═CH₂ | >N— | H | H | —CH═CH—CH═CH— | —CH═CH—CH═CH— |
| Compound 978 | >C═CH₂ | H | >C═CH₂ | >N— | H | H | | |
| Compound 979 | >C═CH₂ | H | >C═CH₂ | >N— | H | H | | |
| Compound 980 | >C═CH₂ | H | >C═CH₂ | >N— | H | H | —CH═CH—CH═CH— | |
| Compound 981 | >C═NH | H | >C═NH | >N— | H | H | —CH═CH—CH═CH— | H |
| Compound 982 | >C═NH | H | >C═NH | >N— | H | H | —CH═CH—CH═CH— | —CH═CH—CH═CH— |
| Compound 983 | >C═NH | H | >C═NH | >N— | H | H | | |
| Compound 984 | >C═NH | H | >C═NH | >N— | H | H | | |
| Compound 985 | >C═NH | H | >C═NH | >N— | H | H | —CH═CH—CH═CH— | |
| Compound 986 | >NCH₃ | H | >NCH₃ | >N— | H | H | —CH═CH—CH═CH— | H |
| Compound 987 | >NCH₃ | H | >NCH₃ | >N— | H | H | —CH═CH—CH═CH— | —CH═CH—CH═CH— |
| Compound 988 | >NCH₃ | H | >NCH₃ | >N— | H | H | | |
| Compound 989 | >NCH₃ | H | >NCH₃ | >N— | H | H | | |
| Compound 990 | >NCH₃ | H | >NCH₃ | >N— | H | H | —CH═CH—CH═CH— | |
| Compound 991 | —O— | H | —O— | >B— | H | H | —CH═CH—CH═CH— | H |
| Compound 992 | —O— | H | —O— | >B— | H | H | —CH═CH—CH═CH— | —CH═CH—CH═CH— |
| Compound 993 | —O— | H | —O— | >B— | H | H | | |
| Compound 994 | —O— | H | —O— | >B— | H | H | | |
| Compound 995 | —O— | H | —O— | >B— | H | H | —CH═CH—CH═CH— | |
| Compound 996 | —O— | H | —O— | >P(═O)— | H | H | —CH═CH—CH═CH— | H |
| Compound 997 | —O— | H | —O— | >P(═O)— | H | H | —CH═CH—CH═CH— | —CH═CH—CH═CH— |
| Compound 998 | —O— | H | —O— | >P(═O)— | H | H | | |
| Compound 999 | —O— | H | —O— | >P(═O)— | H | H | | |
| Compound 1000 | —O— | H | —O— | >P(═O)— | H | H | —CH═CH—CH═CH— | |

| | General Formula [6] | | | | |
|---|---|---|---|---|---|
| Compound | R⁷, R¹⁶, | R⁶, R⁷ and | R¹⁰, R¹³ and | R⁸, R¹², | R⁷, R¹² |
| No. | R²⁵, R¹⁸ | R¹⁴, R¹⁵ | R¹⁶, R¹⁹ | R¹⁸, R²⁰ | R¹⁹, R²⁰ |
| Compound 901 | H | | | | |
| Compound 902 | H | | | | |
| Compound 903 | | —CH═CH—CH═CH— | H | H | |
| Compound 904 | | —CH═CH—CH═CH— | —CH═CH—CH═CH— | H | |
| Compound 905 | | | —CH═CH—CH═CH— | | H |
| Compound 906 | H | | | | |
| Compound 907 | H | | | | |
| Compound 908 | | —CH═CH—CH═CH— | H | H | |
| Compound 909 | | —CH═CH—CH═CH— | —CH═CH—CH═CH— | H | |
| Compound 910 | | | —CH═CH—CH═CH— | | H |
| Compound 911 | H | | | | |
| Compound 912 | H | | | | |
| Compound 913 | | —CH═CH—CH═CH— | H | H | |
| Compound 914 | | —CH═CH—CH═CH— | —CH═CH—CH═CH— | H | |
| Compound 915 | | | —CH═CH—CH═CH— | | H |
| Compound 916 | H | | | | |
| Compound 917 | H | | | | |
| Compound 918 | | —CH═CH—CH═CH— | H | H | |
| Compound 919 | | —CH═CH—CH═CH— | —CH═CH—CH═CH— | H | |
| Compound 920 | | | —CH═CH—CH═CH— | | H |
| Compound 921 | H | | | | |
| Compound 922 | H | | | | |
| Compound 923 | | —CH═CH—CH═CH— | H | H | |
| Compound 924 | | —CH═CH—CH═CH— | —CH═CH—CH═CH— | H | |
| Compound 925 | | | —CH═CH—CH═CH— | | H |
| Compound 926 | H | | | | |
| Compound 927 | H | | | | |
| Compound 928 | | —CH═CH—CH═CH— | H | H | |
| Compound 929 | | —CH═CH—CH═CH— | —CH═CH—CH═CH— | H | |
| Compound 930 | | | —CH═CH—CH═CH— | | H |
| Compound 931 | H | | | | |
| Compound 932 | H | | | | |
| Compound 933 | | —CH═CH—CH═CH— | H | H | |
| Compound 934 | | —CH═CH—CH═CH— | —CH═CH—CH═CH— | H | |
| Compound 935 | | | —CH═CH—CH═CH— | | H |
| Compound 936 | H | | | | |
| Compound 937 | H | | | | |
| Compound 938 | | —CH═CH—CH═CH— | H | H | |
| Compound 939 | | —CH═CH—CH═CH— | —CH═CH—CH═CH— | H | |
| Compound 940 | | | —CH═CH—CH═CH— | | H |
| Compound 941 | H | | | | |
| Compound 942 | H | | | | |
| Compound 943 | | —CH═CH—CH═CH— | H | H | |
| Compound 944 | | —CH═CH—CH═CH— | —CH═CH—CH═CH— | H | |
| Compound 945 | | | —CH═CH—CH═CH— | | H |
| Compound 946 | H | | | | |
| Compound 947 | H | | | | |
| Compound 948 | | —CH═CH—CH═CH— | H | H | |
| Compound 949 | | —CH═CH—CH═CH— | —CH═CH—CH═CH— | H | |
| Compound 950 | | | —CH═CH—CH═CH— | | H |
| Compound 951 | H | | | | |
| Compound 952 | H | | | | |
| Compound 953 | | —CH═CH—CH═CH— | H | H | |
| Compound 954 | | —CH═CH—CH═CH— | —CH═CH—CH═CH— | H | |
| Compound 955 | | | —CH═CH—CH═CH— | | H |
| Compound 956 | H | | | | |
| Compound 957 | H | | | | |
| Compound 958 | | —CH═CH—CH═CH— | H | H | |
| Compound 959 | | —CH═CH—CH═CH— | —CH═CH—CH═CH— | H | |
| Compound 960 | | | —CH═CH—CH═CH— | | H |
| Compound 961 | H | | | | |
| Compound 962 | H | | | | |
| Compound 963 | | —CH═CH—CH═CH— | H | H | |
| Compound 964 | | —CH═CH—CH═CH— | —CH═CH—CH═CH— | H | |
| Compound 965 | | | —CH═CH—CH═CH— | | H |
| Compound 966 | H | | | | |
| Compound 967 | H | | | | |
| Compound 968 | | —CH═CH—CH═CH— | H | H | |
| Compound 969 | | —CH═CH—CH═CH— | —CH═CH—CH═CH— | H | |
| Compound 970 | | | —CH═CH—CH═CH— | | H |
| Compound 971 | H | | | | |
| Compound 972 | H | | | | |
| Compound 973 | | —CH═CH—CH═CH— | H | H | |
| Compound 974 | | —CH═CH—CH═CH— | —CH═CH—CH═CH— | H | |
| Compound 975 | | | —CH═CH—CH═CH— | | H |
| Compound 976 | H | | | | |
| Compound 977 | H | | | | |
| Compound 978 | | —CH═CH—CH═CH— | H | H | |
| Compound 979 | | —CH═CH—CH═CH— | —CH═CH—CH═CH— | H | |
| Compound 980 | | | —CH═CH—CH═CH— | | H |
| Compound 981 | H | | | | |
| Compound 982 | H | | | | |
| Compound 983 | | —CH═CH—CH═CH— | H | H | |
| Compound 984 | | —CH═CH—CH═CH— | —CH═CH—CH═CH— | H | |
| Compound 985 | | | —CH═CH—CH═CH— | | H |
| Compound 986 | H | | | | |
| Compound 987 | H | | | | |
| Compound 988 | | —CH═CH—CH═CH— | H | H | |
| Compound 989 | | —CH═CH—CH═CH— | —CH═CH—CH═CH— | H | |
| Compound 990 | | | —CH═CH—CH═CH— | | H |
| Compound 991 | H | | | | |
| Compound 992 | H | | | | |
| Compound 993 | | —CH═CH—CH═CH— | H | H | |
| Compound 994 | | —CH═CH—CH═CH— | —CH═CH—CH═CH— | H | |
| Compound 995 | | | —CH═CH—CH═CH— | | H |
| Compound 996 | H | | | | |
| Compound 997 | H | | | | |
| Compound 998 | | —CH═CH—CH═CH— | H | H | |
| Compound 999 | | —CH═CH—CH═CH— | —CH═CH—CH═CH— | H | |
| Compound 1000 | | | —CH═CH—CH═CH— | | H |

TABLE 9

| | General Formula [7] | | | | | | | | | | | |
|---|---|---|---|---|---|---|---|---|---|---|---|---|
| | | L¹ and L², | L² and L³, | | | | | | | R¹⁰, | R¹¹, | R¹⁷, |
| Compound | | L⁵ and L⁸, | L⁷ and L⁸, | Y¹, Y² | R¹, R⁴ | R², R³ | R⁵, R¹² | R⁵, R¹⁴ | R⁷, R¹⁵ | R¹⁸ | R¹⁹ | R²⁰ |
| No. | X¹, X⁴, X⁷ | L⁹ and L¹⁰ | L¹¹ and L¹² | Y³ | R⁴⁰ | R⁴¹ | R⁴³ | R⁴⁴ | R⁴⁵ | R⁴⁶ | R⁴⁷ | R⁴⁶ |
| Compound 1001 | —O— | —O— | H | >N— | H | H | H | H | H | H | H | H |
| Compound 1002 | —O— | —O— | H | >N— | H | H | H | H | CH₃O | CH₃O | H | H |
| Compound 1003 | —O— | —O— | H | >N— | H | H | H | H | CH₃O | H | H | H |
| Compound 1004 | —O— | —O— | H | >N— | H | H | H | H | CH₃ | H | H | H |
| Compound 1005 | —O— | —O— | H | >N— | H | H | H | H | C₆H₅O | C₆H₅O | H | H |
| Compound 1006 | —O— | —O— | H | >N— | H | H | H | H | C₆H₅ | C₆H₅ | H | H |
| Compound 1007 | —O— | —O— | H | >N— | H | H | H | H | CF₃O | CF₃O | H | H |
| Compound 1008 | —O— | —O— | H | >N— | H | H | H | H | CF₃O | H | H | H |
| Compound 1009 | —O— | —O— | H | >N— | H | H | H | H | H | H | H | H |
| Compound 1010 | —O— | —O— | H | >N— | H | H | CH₃O | CH₃O | H | H | H | H |
| Compound 1011 | —O— | —O— | H | >N— | H | H | CH₃O | H | H | H | H | H |
| Compound 1012 | —O— | —O— | H | >N— | H | H | CH₃ | H | H | H | H | H |
| Compound 1013 | —O— | —O— | H | >N— | H | H | C₆H₅O | C₆H₅O | H | H | H | H |
| Compound 1014 | —O— | —O— | H | >N— | H | H | C₆H₅ | C₆H₅ | H | H | H | H |
| Compound 1015 | —O— | —O— | H | >N— | H | H | CF₃O | CF₃O | H | H | H | H |
| Compound 1016 | —O— | —O— | H | >N— | H | H | CF₃O | H | H | H | H | H |
| Compound 1017 | —O— | —O— | H | >N— | H | H | H | H | H | H | H | H |
| Compound 1018 | —O— | —O— | H | >N— | H | H | H | H | H | H | CH₃O | CH₃O |
| Compound 1019 | —O— | —O— | H | >N— | H | H | H | H | H | H | CH₃O | H |
| Compound 1020 | —O— | —O— | H | >N— | H | H | H | H | H | H | CH₃ | H |
| Compound 1021 | —O— | —O— | H | >N— | H | H | H | H | H | H | C₆H₅O | C₆H₅O |
| Compound 1022 | —O— | —O— | H | >N— | H | H | H | H | H | H | C₆H₅ | C₆H₅ |
| Compound 1023 | —O— | —O— | H | >N— | H | H | H | H | H | H | CF₃O | CF₃O |
| Compound 1024 | —O— | —O— | H | >N— | H | H | H | H | H | H | CF₃O | H |
| Compound 1025 | —S— | —S— | H | >N— | H | H | H | H | H | H | H | H |
| Compound 1026 | —S— | —S— | H | >N— | H | H | H | H | CH₃O | CH₃O | H | H |
| Compound 1027 | —S— | —S— | H | >N— | H | H | H | H | CH₃O | H | H | H |
| Compound 1028 | —S— | —S— | H | >N— | H | H | H | H | CF₃O | CF₃O | H | H |
| Compound 1029 | —S— | —S— | H | >N— | H | H | H | H | CF₃O | H | H | H |
| Compound 1030 | —SO₂— | —SO₂— | H | >N— | H | H | H | H | H | H | H | H |
| Compound 1031 | —SO₂— | —SO₂— | H | >N— | H | H | H | H | CH₃O | CH₃O | H | H |
| Compound 1032 | —SO₂— | —SO₂— | H | >N— | H | H | H | H | CH₃O | H | H | H |
| Compound 1033 | —SO₂— | —SO₂— | H | >N— | H | H | H | H | CF₃O | CF₃O | H | H |
| Compound 1034 | —SO₂— | —SO₂— | H | >N— | H | H | H | H | CF₃O | H | H | H |
| Compound 1035 | —CH₂— | —CH₂— | H | >N— | H | H | H | H | H | H | H | H |
| Compound 1036 | —CH₂— | —CH₂— | H | >N— | H | H | H | H | CH₃O | CH₃O | H | H |
| Compound 1037 | —CH₂— | —CH₂— | H | >N— | H | H | H | H | CH₃O | H | H | H |
| Compound 1038 | —CH₂— | —CH₂— | H | >N— | H | H | H | H | CF₃O | CF₃O | H | H |
| Compound 1039 | —CH₂— | —CH₂— | H | >N— | H | H | H | H | CF₃O | H | H | H |
| Compound 1040 | >C═O | >C═O | H | >N— | H | H | H | H | H | H | H | H |
| Compound 1041 | >C═O | >C═O | H | >N— | H | H | H | H | CH₃O | CH₃O | H | H |
| Compound 1042 | >C═O | >C═O | H | >N— | H | H | H | H | CH₃O | H | H | H |
| Compound 1043 | >C═O | >C═O | H | >N— | H | H | H | H | CF₃O | CF₃O | H | H |
| Compound 1044 | >C═O | >C═O | H | >N— | H | H | H | H | CF₃O | H | H | H |
| Compound 1045 | >C═CH₂ | >C═CH₂ | H | >N— | H | H | H | H | H | H | H | H |
| Compound 1046 | >C═CH₂ | >C═CH₂ | H | >N— | H | H | H | H | CH₃O | CH₃O | H | H |
| Compound 1047 | >C═CH₂ | >C═CH₂ | H | >N— | H | H | H | H | CH₃O | H | H | H |
| Compound 1048 | >C═CH₂ | >C═CH₂ | H | >N— | H | H | H | H | CF₃O | CF₃O | H | H |
| Compound 1049 | >C═CH₂ | >C═CH₂ | H | >N— | H | H | H | H | CF₃O | H | H | H |
| Compound 1050 | >C═NH | >C═NH | H | >N— | H | H | H | H | H | H | H | H |
| Compound 1051 | >C═NH | >C═NH | H | >N— | H | H | H | H | CH₃O | CH₃O | H | H |
| Compound 1052 | >C═NH | >C═NH | H | >N— | H | H | H | H | CH₃O | H | H | H |
| Compound 1053 | >C═NH | >C═NH | H | >N— | H | H | H | H | CF₃O | CF₃O | H | H |
| Compound 1054 | >C═NH | >C═NH | H | >N— | H | H | H | H | CF₃O | H | H | H |
| Compound 1055 | >NCH₃ | >NCH₃ | H | >N— | H | H | H | H | H | H | H | H |
| Compound 1056 | >NCH₃ | >NCH₃ | H | >N— | H | H | H | H | CH₃O | CH₃O | H | H |
| Compound 1057 | >NCH₃ | >NCH₃ | H | >N— | H | H | H | H | CH₃O | H | H | H |
| Compound 1058 | >NCH₃ | >NCH₃ | H | >N— | H | H | H | H | CF₃O | CF₃O | H | H |
| Compound 1059 | >NCH₃ | >NCH₃ | H | >N— | H | H | H | H | CF₃O | H | H | H |
| Compound 1060 | —O— | —O— | H | >B— | H | H | H | H | H | H | H | H |
| Compound 1061 | —O— | —O— | H | >B— | H | H | H | H | CH₃O | CH₃O | H | H |
| Compound 1062 | —O— | —O— | H | >B— | H | H | H | H | CH₃O | H | H | H |
| Compound 1063 | —O— | —O— | H | >B— | H | H | H | H | CF₃O | CF₃O | H | H |
| Compound 1064 | —O— | —O— | H | >B— | H | H | H | H | CF₃O | H | H | H |
| Compound 1065 | —O— | —O— | H | >P(═O)— | H | H | H | H | H | H | H | H |
| Compound 1066 | —O— | —O— | H | >P(═O)— | H | H | H | H | CH₃O | CH₃O | H | H |
| Compound 1067 | —O— | —O— | H | >P(═O)— | H | H | H | H | CH₃O | H | H | H |
| Compound 1068 | —O— | —O— | H | >P(═O)— | H | H | H | H | CF₃O | CF₃O | H | H |
| Compound 1069 | —O— | —O— | H | >P(═O)— | H | H | H | H | CF₃O | H | H | H |
| Compound 1070 | —O— | H | —O— | >N— | H | H | H | H | H | H | H | H |
| Compound 1071 | —O— | H | —O— | >N— | H | H | H | H | CH₃O | CH₃O | H | H |
| Compound 1072 | —O— | H | —O— | >N— | H | H | H | H | CH₃O | H | H | H |
| Compound 1073 | —O— | H | —O— | >N— | H | H | H | H | CH₃ | H | H | H |
| Compound 1074 | —O— | H | —O— | >N— | H | H | H | H | C₆H₅O | C₆H₅O | H | H |
| Compound 1075 | —O— | H | —O— | >N— | H | H | H | H | C₆H₅ | C₆H₅ | H | H |
| Compound 1076 | —O— | H | —O— | >N— | H | H | H | H | CF₃O | CF₃O | H | H |
| Compound 1077 | —O— | H | —O— | >N— | H | H | H | H | CF₃O | H | H | H |
| Compound 1078 | —O— | H | —O— | >N— | H | H | H | H | H | H | H | H |
| Compound 1079 | —O— | H | —O— | >N— | H | H | CH₃O | CH₃O | H | H | H | H |
| Compound 1080 | —O— | H | —O— | >N— | H | H | CH₃O | H | H | H | H | H |
| Compound 1081 | —O— | H | —O— | >N— | H | H | CH₃ | H | H | H | H | H |
| Compound 1082 | —O— | H | —O— | >N— | H | H | C₆H₅O | C₆H₅O | H | H | H | H |
| Compound 1083 | —O— | H | —O— | >N— | H | H | C₆H₅ | C₆H₅ | H | H | H | H |
| Compound 1084 | —O— | H | —O— | >N— | H | H | CF₃O | CF₃O | H | H | H | H |
| Compound 1085 | —O— | H | —O— | >N— | H | H | CF₃O | H | H | H | H | H |
| Compound 1086 | —O— | H | —O— | >N— | H | H | H | H | H | H | H | H |
| Compound 1087 | —O— | H | —O— | >N— | H | H | H | H | H | H | CH₃O | CH₃O |
| Compound 1088 | —O— | H | —O— | >N— | H | H | H | H | H | H | CH₃O | H |
| Compound 1089 | —O— | H | —O— | >N— | H | H | H | H | H | H | CH₂ | H |
| Compound 1090 | —O— | H | —O— | >N— | H | H | H | H | H | H | C₆H₅O | C₆H₅O |
| Compound 1091 | —O— | H | —O— | >N— | H | H | H | H | H | H | C₆H₅ | C₆H₅ |
| Compound 1092 | —O— | H | —O— | >N— | H | H | H | H | H | H | CF₃O | CF₃O |
| Compound 1093 | —O— | H | —O— | >N— | H | H | H | H | H | H | CF₃O | H |
| Compound 1094 | —S— | H | —S— | >N— | H | H | H | H | H | H | H | H |
| Compound 1095 | —S— | H | —S— | >N— | H | H | H | H | CH₃O | CH₃O | H | H |
| Compound 1096 | —S— | H | —S— | >N— | H | H | H | H | CH₃O | H | H | H |
| Compound 1097 | —S— | H | —S— | >N— | H | H | H | H | CF₃O | CF₃O | H | H |
| Compound 1098 | —S— | H | —S— | >N— | H | H | H | H | CF₃O | H | H | H |
| Compound 1099 | —SO₂— | H | —SO₂— | >N— | H | H | H | H | H | H | H | H |
| Compound 1100 | —SO₂— | H | —SO₂— | >N— | H | H | H | H | CH₃O | CH₃O | H | H |
| Compound 1101 | —SO₂— | H | —SO₂— | >N— | H | H | H | H | CH₃O | H | H | H |
| Compound 1102 | —SO₂— | H | —SO₂— | >N— | H | H | H | H | CF₃O | CF₃O | H | H |
| Compound 1103 | —SO₂— | H | —SO₂— | >N— | H | H | H | H | CF₃O | H | H | H |
| Compound 1104 | —CH₂— | H | —CH₂— | >N— | H | H | H | H | H | H | H | H |
| Compound 1105 | —CH₂— | H | —CH₂— | >N— | H | H | H | H | CH₃O | CH₃O | H | H |
| Compound 1106 | —CH₂— | H | —CH₂— | >N— | H | H | H | H | CH₃O | H | H | H |
| Compound 1107 | —CH₂— | H | —CH₂— | >N— | H | H | H | H | CF₃O | CF₃O | H | H |
| Compound 1108 | —CH₂— | H | —CH₂— | >N— | H | H | H | H | CF₃O | H | H | H |
| Compound 1109 | >C═O | H | >C═O | >N— | H | H | H | H | H | H | H | H |
| Compound 1110 | >C═O | H | >C═O | >N— | H | H | H | H | CH₃O | CH₃O | H | H |
| Compound 1111 | >C═O | H | >C═O | >N— | H | H | H | H | CH₃O | H | H | H |
| Compound 1112 | >C═O | H | >C═O | >N— | H | H | H | H | CF₃O | CF₃O | H | H |
| Compound 1113 | >C═O | H | >C═O | >N— | H | H | H | H | CF₃O | H | H | H |
| Compound 1114 | >C═CH₂ | H | >C═CH₂ | >N— | H | H | H | H | H | H | H | H |
| Compound 1115 | >C═CH₂ | H | >C═CH₂ | >N— | H | H | H | H | CH₃O | CH₃O | H | H |
| Compound 1116 | >C═CH₂ | H | >C═CH₂ | >N— | H | H | H | H | CH₃O | H | H | H |
| Compound 1117 | >C═CH₂ | H | >C═CH₂ | >N— | H | H | H | H | CF₃O | CF₃O | H | H |
| Compound 1118 | >C═CH₂ | H | >C═CH₂ | >N— | H | H | H | H | CF₃O | H | H | H |
| Compound 1119 | >C═NH | H | >C═NH | >N— | H | H | H | H | H | H | H | H |
| Compound 1120 | >C═NH | H | >C═NH | >N— | H | H | H | H | CH₃O | CH₃O | H | H |
| Compound 1121 | >C═NH | H | >C═NH | >N— | H | H | H | H | CH₃O | H | H | H |
| Compound 1122 | >C═NH | H | >C═NH | >N— | H | H | H | H | CF₃O | CF₃O | H | H |
| Compound 1123 | >C═NH | H | >C═NH | >N— | H | H | H | H | CF₃O | H | H | H |
| Compound 1124 | >NCH₃ | H | >NCH₃ | >N— | H | H | H | H | H | H | H | H |
| Compound 1125 | >NCH₃ | H | >NCH₃ | >N— | H | H | H | H | CH₃O | CH₃O | H | H |
| Compound 1126 | >NCH₃ | H | >NCH₃ | >N— | H | H | H | H | CH₃O | H | H | H |
| Compound 1127 | >NCH₃ | H | >NCH₃ | >N— | H | H | H | H | CF₃O | CF₃O | H | H |
| Compound 1128 | >NCH₃ | H | >NCH₃ | >N— | H | H | H | H | CF₃O | H | H | H |
| Compound 1129 | —O— | H | —O— | >B— | H | H | H | H | H | H | H | H |
| Compound 1130 | —O— | H | —O— | >B— | H | H | H | H | CH₃O | CH₃O | H | H |
| Compound 1131 | —O— | H | —O— | >B— | H | H | H | H | CH₃O | H | H | H |
| Compound 1132 | —O— | H | —O— | >B— | H | H | H | H | CF₃O | CF₃O | H | H |
| Compound 1133 | —O— | H | —O— | >B— | H | H | H | H | CF₃O | H | H | H |
| Compound 1134 | —O— | H | —O— | >P(═O)— | H | H | H | H | H | H | H | H |
| Compound 1135 | —O— | H | —O— | >P(═O)— | H | H | H | H | CH₃O | CH₃O | H | H |
| Compound 1136 | —O— | H | —O— | >P(═O)— | H | H | H | H | CH₃O | H | H | H |
| Compound 1137 | —O— | H | —O— | >P(═O)— | H | H | H | H | CF₃O | CF₃O | H | H |
| Compound 1138 | —O— | H | —O— | >P(═O)— | H | H | H | H | CF₃O | H | H | H |
| Compound 1139 | —O— | —O— | H | >N— | H | H | CF₃ | H | H | H | H | H |
| Compound 1140 | —O— | —O— | H | >N— | H | H | H | CF₃ | H | H | H | H |
| Compound 1141 | —O— | —O— | H | >N— | H | H | H | H | CF₃ | H | H | H |
| Compound 1142 | —O— | —O— | H | >N— | H | H | H | H | H | CF₃ | H | H |
| Compound 1143 | —O— | —O— | H | >N— | H | H | H | H | H | H | CF₃ | H |
| Compound 1144 | —O— | —O— | H | >N— | H | H | H | H | H | H | H | CF₃ |
| Compound 1145 | —O— | —O— | H | >N— | H | H | CF₃ | CF₃ | H | H | H | H |
| Compound 1146 | —O— | —O— | H | >N— | H | H | CF₃ | H | CF₃ | H | H | H |
| Compound 1147 | —O— | —O— | H | >N— | H | H | CF₃ | H | H | CF₃ | H | H |
| Compound 1148 | —O— | —O— | H | >N— | H | H | CF₃ | H | H | H | CF₃ | H |
| Compound 1149 | —O— | —O— | H | >N— | H | H | CF₃ | H | H | H | H | CF₃ |
| Compound 1150 | —O— | —O— | H | >N— | H | H | H | CF₃ | CF₃ | H | H | H |
| Compound 1151 | —O— | —O— | H | >N— | H | H | H | CF₃ | H | CF₃ | H | H |
| Compound 1152 | —O— | —O— | H | >N— | H | H | H | CF₃ | H | H | CF₃ | H |
| Compound 1153 | —O— | —O— | H | >N— | H | H | H | CF₃ | H | H | H | CF₃ |
| Compound 1154 | —O— | —O— | H | >N— | H | H | H | H | CF₃ | CF₃ | H | H |
| Compound 1155 | —O— | —O— | H | >N— | H | H | H | H | CF₃ | H | CF₃ | H |
| Compound 1156 | —O— | —O— | H | >N— | H | H | H | H | CF₃ | H | H | CF₃ |
| Compound 1157 | —O— | —O— | H | >N— | H | H | H | H | H | CF₃ | CF₃ | H |
| Compound 1158 | —O— | —O— | H | >N— | H | H | H | H | H | CF₃ | H | CF₃ |
| Compound 1159 | —O— | —O— | H | >N— | H | H | H | H | H | H | CF₃ | CF₃ |
| Compound 1160 | —O— | H | —O— | >N— | H | H | CF₃ | H | H | H | H | H |
| Compound 1160 | —O— | H | —O— | >N— | H | H | H | CF₃ | H | H | H | H |
| Compound 1162 | —O— | H | —O— | >N— | H | H | H | H | CF₃ | H | H | H |
| Compound 1163 | —O— | H | —O— | >N— | H | H | H | H | H | CF₃ | H | H |
| Compound 1164 | —O— | H | —O— | >N— | H | H | H | H | H | H | CF₃ | H |
| Compound 1165 | —O— | H | —O— | >N— | H | H | H | H | H | H | H | CF₃ |
| Compound 1166 | —O— | H | —O— | >N— | H | H | CF₃ | CF₃ | H | H | H | H |
| Compound 1167 | —O— | H | —O— | >N— | H | H | CF₃ | H | CF₃ | H | H | H |
| Compound 1168 | —O— | H | —O— | >N— | H | H | CF₃ | H | H | CF₃ | H | H |
| Compound 1169 | —O— | H | —O— | >N— | H | H | CF₃ | H | H | H | CF₃ | H |
| Compound 1170 | —O— | H | —O— | >N— | H | H | CF₃ | H | H | H | H | CF₃ |
| Compound 1171 | —O— | H | —O— | >N— | H | H | H | CF₃ | CF₃ | H | H | H |
| Compound 1172 | —O— | H | —O— | >N— | H | H | H | CF₃ | H | CF₃ | H | H |
| Compound 1173 | —O— | H | —O— | >N— | H | H | H | CF₃ | H | H | CF₃ | H |
| Compound 1174 | —O— | H | —O— | >N— | H | H | H | CF₃ | H | H | H | CF₃ |
| Compound 1175 | —O— | H | —O— | >N— | H | H | H | H | CF₃ | CF₃ | H | H |
| Compound 1176 | —O— | H | —O— | >N— | H | H | H | H | CF₃ | H | CF₃ | H |
| Compound 1177 | —O— | H | —O— | >N— | H | H | H | H | CF₃ | H | H | CF₃ |
| Compound 1178 | —O— | H | —O— | >N— | H | H | H | H | H | CF₃ | CF₃ | H |
| Compound 1179 | —O— | H | —O— | >N— | H | H | H | H | H | CF₃ | H | CF₃ |
| Compound 1180 | —O— | H | —O— | >N— | H | H | H | H | H | H | CF₃ | CF₃ |

TABLE 10

| | General Formula [7] | | | | | | | |
|---|---|---|---|---|---|---|---|---|
| | | L¹ and L², | L³ and L⁴, | | | | R⁵, R⁶ and | R¹¹, R¹² and |
| Compound | | L³ and L⁴, | L⁷ and L⁹, | Y¹, Y² | R¹, R⁴ | R², R³ | R¹⁹, R²⁰ and | R¹³, R¹⁴ and |
| No. | X¹, X⁴, X⁷ | L⁹ and L¹⁹ | L²³ and L¹³ | Y³ | R⁴² | R⁴¹ | R⁴⁹, R⁵⁰ | R⁴³, R⁴⁴ |
| Compound 1201 | —O— | —O— | H | >N— | H | H | —CH═CH—CH═CH— | H |
| Compound 1202 | —O— | —O— | H | >N— | H | H | —CH═CH—CH═CH— | —CH═CH—CH═CH— |
| Compound 1203 | —O— | —O— | H | >N— | H | H | | |
| Compound 1204 | —O— | —O— | H | >N— | H | H | | |
| Compound 1205 | —O— | —O— | H | >N— | H | H | —CH═CH—CH═CH— | |
| Compound 1206 | —S— | —S— | H | >N— | H | H | —CH═CH—CH═CH— | H |
| Compound 1207 | —S— | —S— | H | >N— | H | H | —CH═CH—CH═CH— | —CH═CH—CH═CH— |
| Compound 1208 | —S— | —S— | H | >N— | H | H | | |
| Compound 1209 | —S— | —S— | H | >N— | H | H | | |
| Compound 1210 | —S— | —S— | H | >N— | H | H | —CH═CH—CH═CH— | |
| Compound 1211 | —SO₂— | —SO₂— | H | >N— | H | H | —CH═CH—CH═CH— | H |
| Compound 1212 | —SO₂— | —SO₂— | H | >N— | H | H | —CH═CH—CH═CH— | —CH═CH—CH═CH— |
| Compound 1213 | —SO₂— | —SO₂— | H | >N— | H | H | | |
| Compound 1214 | —SO₂— | —SO₂— | H | >N— | H | H | | |
| Compound 1215 | —SO₂— | —SO₂— | H | >N— | H | H | —CH═CH—CH═CH— | |
| Compound 1216 | —CH₂— | —CH₂— | H | >N— | H | H | —CH═CH—CH═CH— | H |
| Compound 1217 | —CH₂— | —CH₂— | H | >N— | H | H | —CH═CH—CH═CH— | —CH═CH—CH═CH— |
| Compound 1218 | —CH₂— | —CH₂— | H | >N— | H | H | | |
| Compound 1219 | —CH₂— | —CH₂— | H | >N— | H | H | | |
| Compound 1220 | —CH₂— | —CH₂— | H | >N— | H | H | —CH═CH—CH═CH— | |
| Compound 1221 | >C═O | >C═O | H | >N— | H | H | —CH═CH—CH═CH— | H |
| Compound 1222 | >C═O | >C═O | H | >N— | H | H | —CH═CH—CH═CH— | —CH═CH—CH═CH— |
| Compound 1223 | >C═O | >C═O | H | >N— | H | H | | |
| Compound 1224 | >C═O | >C═O | H | >N— | H | H | | |
| Compound 1225 | >C═O | >C═O | H | >N— | H | H | —CH═CH—CH═CH— | |
| Compound 1226 | >C═CH₂ | >C═CH₂ | H | >N— | H | H | —CH═CH—CH═CH— | H |
| Compound 1227 | >C═CH₂ | >C═CH₂ | H | >N— | H | H | —CH═CH—CH═CH— | —CH═CH—CH═CH— |
| Compound 1228 | >C═CH₂ | >C═CH₂ | H | >N— | H | H | | |
| Compound 1229 | >C═CH₂ | >C═CH₂ | H | >N— | H | H | | |
| Compound 1230 | >C═CH₂ | >C═CH₂ | H | >N— | H | H | —CH═CH—CH═CH— | |
| Compound 1231 | >C═NH | >C═NH | H | >N— | H | H | —CH═CH—CH═CH— | H |
| Compound 1232 | >C═NH | >C═NH | H | >N— | H | H | —CH═CH—CH═CH— | —CH═CH—CH═CH— |
| Compound 1233 | >C═NH | >C═NH | H | >N— | H | H | | |
| Compound 1234 | >C═NH | >C═NH | H | >N— | H | H | | |
| Compound 1235 | >C═NH | >C═NH | H | >N— | H | H | —CH═CH—CH═CH— | |
| Compound 1236 | >NCH₃ | >NCH₃ | H | >N— | H | H | —CH═CH—CH═CH— | H |
| Compound 1237 | >NCH₃ | >NCH₃ | H | >N— | H | H | —CH═CH—CH═CH— | —CH═CH—CH═CH— |
| Compound 1238 | >NCH₃ | >NCH₃ | H | >N— | H | H | | |
| Compound 1239 | >NCH₃ | >NCH₃ | H | >N— | H | H | | |
| Compound 1240 | >NCH₃ | >NCH₃ | H | >N— | H | H | —CH═CH—CH═CH— | |
| Compound 1241 | —O— | —O— | H | >B— | H | H | —CH═CH—CH═CH— | H |
| Compound 1242 | —O— | —O— | H | >B— | H | H | —CH═CH—CH═CH— | —CH═CH—CH═CH— |
| Compound 1243 | —O— | —O— | H | >B— | H | H | | |
| Compound 1244 | —O— | —O— | H | >B— | H | H | | |
| Compound 1245 | —O— | —O— | H | >B— | H | H | —CH═CH—CH═CH— | |
| Compound 1246 | —O— | —O— | H | >P(50 O)— | H | H | —CH═CH—CH═CH— | H |
| Compound 1247 | —O— | —O— | H | >P(50 O)— | H | H | —CH═CH—CH═CH— | —CH═CH—CH═CH— |
| Compound 1248 | —O— | —O— | H | >P(50 O)— | H | H | | |
| Compound 1249 | —O— | —O— | H | >P(50 O)— | H | H | | |
| Compound 1250 | —O— | —O— | H | >P(50 O)— | H | H | —CH═CH—CH═CH— | |
| Compound 1251 | —O— | H | —O— | >N— | H | H | —CH═CH—CH═CH— | H |
| Compound 1252 | —O— | H | —O— | >N— | H | H | —CH═CH—CH═CH— | —CH═CH—CH═CH— |
| Compound 1253 | —O— | H | —O— | >N— | H | H | | |
| Compound 1254 | —O— | H | —O— | >N— | H | H | | |
| Compound 1255 | —O— | H | —O— | >N— | H | H | —CH═CH—CH═CH— | |
| Compound 1256 | —S— | H | —S— | >N— | H | H | —CH═CH—CH═CH— | H |
| Compound 1257 | —S— | H | —S— | >N— | H | H | —CH═CH—CH═CH— | —CH═CH—CH═CH— |
| Compound 1258 | —S— | H | —S— | >N— | H | H | | |
| Compound 1259 | —S— | H | —S— | >N— | H | H | | |
| Compound 1260 | —S— | H | —S— | >N— | H | H | —CH═CH—CH═CH— | |
| Compound 1261 | —SO₂— | H | —SO₂— | >N— | H | H | —CH═CH—CH═CH— | H |
| Compound 1262 | —SO₂— | H | —SO₂— | >N— | H | H | —CH═CH—CH═CH— | —CH═CH—CH═CH— |
| Compound 1263 | —SO₂— | H | —SO₂— | >N— | H | H | | |
| Compound 1264 | —SO₂— | H | —SO₂— | >N— | H | H | | |
| Compound 1265 | —SO₂— | H | —SO₂— | >N— | H | H | —CH═CH—CH═CH— | |
| Compound 1266 | —CH₂— | H | —CH₂— | >N— | H | H | —CH═CH—CH═CH— | H |
| Compound 1267 | —CH₂— | H | —CH₂— | >N— | H | H | —CH═CH—CH═CH— | —CH═CH—CH═CH— |
| Compound 1268 | —CH₂— | H | —CH₂— | >N— | H | H | | |
| Compound 1269 | —CH₂— | H | —CH₂— | >N— | H | H | | |
| Compound 1270 | —CH₂— | H | —CH₂— | >N— | H | H | —CH═CH—CH═CH— | |
| Compound 1271 | >C═O | H | >C═O | >N— | H | H | —CH═CH—CH═CH— | H |
| Compound 1272 | >C═O | H | >C═O | >N— | H | H | —CH═CH—CH═CH— | —CH═CH—CH═CH— |
| Compound 1273 | >C═O | H | >C═O | >N— | H | H | | |
| Compound 1274 | >C═O | H | >C═O | >N— | H | H | | |
| Compound 1275 | >C═O | H | >C═O | >N— | H | H | —CH═CH—CH═CH— | |
| Compound 1276 | >C═CH₂ | H | >C═CH₂ | >N— | H | H | —CH═CH—CH═CH— | H |
| Compound 1277 | >C═CH₂ | H | >C═CH₂ | >N— | H | H | —CH═CH—CH═CH— | —CH═CH—CH═CH— |
| Compound 1278 | >C═CH₂ | H | >C═CH₂ | >N— | H | H | | |
| Compound 1279 | >C═CH₂ | H | >C═CH₂ | >N— | H | H | | |
| Compound 1280 | >C═CH₂ | H | >C═CH₂ | >N— | H | H | —CH═CH—CH═CH— | |
| Compound 1281 | >C═NH | H | >C═NH | >N— | H | H | —CH═CH—CH═CH— | H |
| Compound 1282 | >C═NH | H | >C═NH | >N— | H | H | —CH═CH—CH═CH— | —CH═CH—CH═CH— |
| Compound 1283 | >C═NH | H | >C═NH | >N— | H | H | | |
| Compound 1284 | >C═NH | H | >C═NH | >N— | H | H | | |
| Compound 1285 | >C═NH | H | >C═NH | >N— | H | H | —CH═CH—CH═CH— | |
| Compound 1286 | >NCH₃ | H | >NCH₃ | >N— | H | H | —CH═CH—CH═CH— | H |
| Compound 1287 | >NCH₃ | H | >NCH₃ | >N— | H | H | —CH═CH—CH═CH— | —CH═CH—CH═CH— |
| Compound 1288 | >NCH₃ | H | >NCH₃ | >N— | H | H | | |
| Compound 1289 | >NCH₃ | H | >NCH₃ | >N— | H | H | | |
| Compound 1290 | >NCH₃ | H | >NCH₃ | >N— | H | H | —CH═CH—CH═CH— | |
| Compound 1291 | —O— | H | —O— | >B— | H | H | —CH═CH—CH═CH— | H |
| Compound 1292 | —O— | H | —O— | >B— | H | H | —CH═CH—CH═CH— | —CH═CH—CH═CH— |
| Compound 1293 | —O— | H | —O— | >B— | H | H | | |
| Compound 1294 | —O— | H | —O— | >B— | H | H | | |
| Compound 1295 | —O— | H | —O— | >B— | H | H | —CH═CH—CH═CH— | |
| Compound 1296 | —O— | H | —O— | >P(═O)— | H | H | —CH═CH—CH═CH— | H |
| Compound 1297 | —O— | H | —O— | >P(═O)— | H | H | —CH═CH—CH═CH— | —CH═CH—CH═CH— |
| Compound 1298 | —O— | H | —O— | >P(═O)— | H | H | | |
| Compound 1299 | —O— | H | —O— | >P(═O)— | H | H | | |
| Compound 1300 | —O— | H | —O— | >P(═O)— | H | H | —CH═CH—CH═CH— | |

| | General Formula [7] | | | | |
|---|---|---|---|---|---|
| | R⁷, R¹⁰ | R⁶, R⁷ and | R¹⁰, R¹³ and | R⁹, R¹² | R⁷, R¹² |
| Compound | R¹³, R¹⁴ | R¹⁸, R¹⁹ and | R¹⁴, R¹⁵ and | R¹², R²³ | R¹², R¹³ |
| No. | R⁴⁸, R⁴⁹ | R⁴³, R⁴⁵ | R⁴⁴, R⁴⁵ | R⁴³, R³⁶ | R⁴³, R⁴⁹ |
| Compound 1201 | H | | | | |
| Compound 1202 | H | | | | |
| Compound 1203 | | —CH═CH—CH═CH— | H | H | |
| Compound 1204 | | —CH═CH—CH═CH— | —CH═CH—CH═CH— | H | |
| Compound 1205 | | | —CH═CH—CH═CH— | | H |
| Compound 1206 | H | | | | |
| Compound 1207 | H | | | | |
| Compound 1208 | | —CH═CH—CH═CH— | H | H | |
| Compound 1209 | | —CH═CH—CH═CH— | —CH═CH—CH═CH— | H | |
| Compound 1210 | | | —CH═CH—CH═CH— | | H |
| Compound 1211 | H | | | | |
| Compound 1212 | H | | | | |
| Compound 1213 | | —CH═CH—CH═CH— | H | H | |
| Compound 1214 | | —CH═CH—CH═CH— | —CH═CH—CH═CH— | H | |
| Compound 1215 | | | —CH═CH—CH═CH— | | H |
| Compound 1216 | H | | | | |
| Compound 1217 | H | | | | |
| Compound 1218 | | —CH═CH—CH═CH— | H | H | |
| Compound 1219 | | —CH═CH—CH═CH— | —CH═CH—CH═CH— | H | |
| Compound 1220 | | | —CH═CH—CH═CH— | | H |
| Compound 1221 | H | | | | |
| Compound 1222 | H | | | | |
| Compound 1223 | | —CH═CH—CH═CH— | H | H | |
| Compound 1224 | | —CH═CH—CH═CH— | —CH═CH—CH═CH— | H | |
| Compound 1225 | | | —CH═CH—CH═CH— | | H |
| Compound 1226 | H | | | | |
| Compound 1227 | H | | | | |
| Compound 1228 | | —CH═CH—CH═CH— | H | H | |
| Compound 1229 | | —CH═CH—CH═CH— | —CH═CH—CH═CH— | H | |
| Compound 1230 | | | —CH═CH—CH═CH— | | H |
| Compound 1231 | H | | | | |
| Compound 1232 | H | | | | |
| Compound 1233 | | —CH═CH—CH═CH— | H | H | |
| Compound 1234 | | —CH═CH—CH═CH— | —CH═CH—CH═CH— | H | |
| Compound 1235 | | | —CH═CH—CH═CH— | | H |
| Compound 1236 | H | | | | |
| Compound 1237 | H | | | | |
| Compound 1238 | | —CH═CH—CH═CH— | H | H | |
| Compound 1239 | | —CH═CH—CH═CH— | —CH═CH—CH═CH— | H | |
| Compound 1240 | | | —CH═CH—CH═CH— | | H |
| Compound 1241 | H | | | | |
| Compound 1242 | H | | | | |
| Compound 1243 | | —CH═CH—CH═CH— | H | H | |
| Compound 1244 | | —CH═CH—CH═CH— | —CH═CH—CH═CH— | H | |
| Compound 1245 | | | —CH═CH—CH═CH— | | H |
| Compound 1246 | H | | | | |
| Compound 1247 | H | | | | |
| Compound 1248 | | —CH═CH—CH═CH— | H | H | |
| Compound 1249 | | —CH═CH—CH═CH— | —CH═CH—CH═CH— | H | |
| Compound 1250 | | | —CH═CH—CH═CH— | | H |
| Compound 1251 | H | | | | |
| Compound 1252 | H | | | | |
| Compound 1253 | | —CH═CH—CH═CH— | H | H | |
| Compound 1254 | | —CH═CH—CH═CH— | —CH═CH—CH═CH— | H | |
| Compound 1255 | | | —CH═CH—CH═CH— | | H |
| Compound 1256 | H | | | | |
| Compound 1257 | H | | | | |
| Compound 1258 | | —CH═CH—CH═CH— | H | H | |
| Compound 1259 | | —CH═CH—CH═CH— | —CH═CH—CH═CH— | H | |
| Compound 1260 | | | —CH═CH—CH═CH— | | H |
| Compound 1261 | H | | | | |
| Compound 1262 | H | | | | |
| Compound 1263 | | —CH═CH—CH═CH— | H | H | |
| Compound 1264 | | —CH═CH—CH═CH— | —CH═CH—CH═CH— | H | |
| Compound 1265 | | | —CH═CH—CH═CH— | | H |
| Compound 1266 | H | | | | |
| Compound 1267 | H | | | | |
| Compound 1268 | | —CH═CH—CH═CH— | H | H | |
| Compound 1269 | | —CH═CH—CH═CH— | —CH═CH—CH═CH— | H | |
| Compound 1270 | | | —CH═CH—CH═CH— | | H |
| Compound 1271 | H | | | | |
| Compound 1272 | H | | | | |
| Compound 1273 | | —CH═CH—CH═CH— | H | H | |
| Compound 1274 | | —CH═CH—CH═CH— | —CH═CH—CH═CH— | H | |
| Compound 1275 | | | —CH═CH—CH═CH— | | H |
| Compound 1276 | H | | | | |
| Compound 1277 | H | | | | |
| Compound 1278 | | —CH═CH—CH═CH— | H | H | |
| Compound 1279 | | —CH═CH—CH═CH— | —CH═CH—CH═CH— | H | |
| Compound 1280 | | | —CH═CH—CH═CH— | | H |
| Compound 1281 | H | | | | |
| Compound 1282 | H | | | | |
| Compound 1283 | | —CH═CH—CH═CH— | H | H | |
| Compound 1284 | | —CH═CH—CH═CH— | —CH═CH—CH═CH— | H | |
| Compound 1285 | | | —CH═CH—CH═CH— | | H |
| Compound 1286 | H | | | | |
| Compound 1287 | H | | | | |
| Compound 1288 | | —CH═CH—CH═CH— | H | H | |
| Compound 1289 | | —CH═CH—CH═CH— | —CH═CH—CH═CH— | H | |
| Compound 1290 | | | —CH═CH—CH═CH— | | H |
| Compound 1291 | H | | | | |
| Compound 1292 | H | | | | |
| Compound 1293 | | —CH═CH—CH═CH— | H | H | |
| Compound 1294 | | —CH═CH—CH═CH— | —CH═CH—CH═CH— | H | |
| Compound 1295 | | | —CH═CH—CH═CH— | | H |
| Compound 1296 | H | | | | |
| Compound 1297 | H | | | | |
| Compound 1298 | | —CH═CH—CH═CH— | H | H | |
| Compound 1299 | | —CH═CH—CH═CH— | —CH═CH—CH═CH— | H | |
| Compound 1300 | | | —CH═CH—CH═CH— | | H |

### [Synthesis of Compounds Represented by General Formula [1]]

The production method for the compound represented by the general formula [1] is not specifically defined. The compound represented by the general formula [1] may be produced by suitably combining some known production methods and conditions.

For example, as one preferred production method, there may be mentioned, a production method represented by the following scheme 1. Here the method is described as a production scheme for the compound represented by the general formula [2].

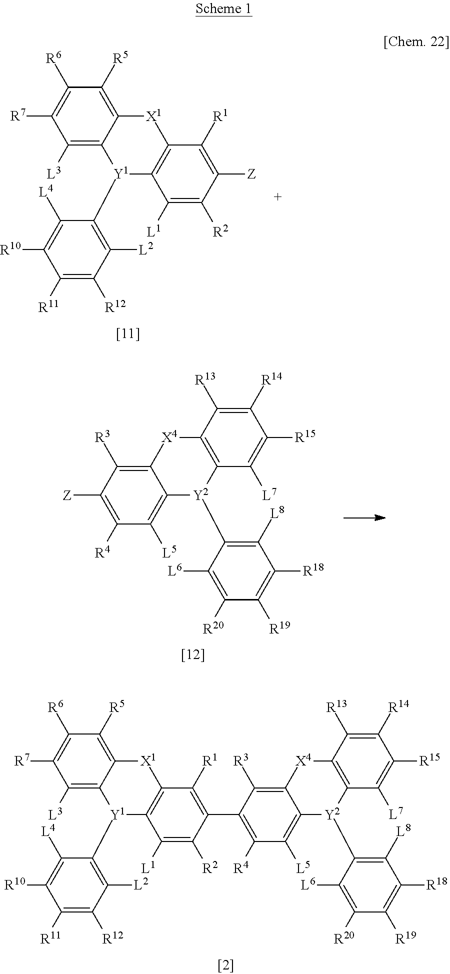

The definitions of X¹, X⁴, Y¹, Y², L¹ to L⁸, R⁵ to R⁷, R¹⁰ to R¹², R¹³ to R¹⁵ and R¹⁸ to R²⁰ in the general formula [11] and the general formula [12] are the same as in the general formulae [1] and [2]. R¹ to R⁴ in the general formula [11] and the general formula [12] each represent a hydrogen atom or a substituent, and the descriptions and the preferred ranges of the substituent are the same as the descriptions and the preferred, ranges of the substituent for R¹ and R² in the general formula [1]. Z in the general formula [11] and the general formula [12] represents a halogen atom, and is preferably a fluorine atom, a chlorine atom, a bromine atom or an iodine atom, more preferably a chlorine atom, a bromine atom or an iodine atom, even more preferably a bromine atom.

The reaction of the scheme 1 is a coupling reaction, for which, in general, a coupling agent is used. Specifically, Z in the general formula [12] is metallized and the resulting compound is reacted in a mode of known cross coupling reaction using palladium(0) or nickel(0) to give the compound represented by the general, formula [1]. The reaction condition can be optimized with reference to known conditions.

In case where a compound having a bilaterally-symmetric molecular structure is produced as the compound of the general formula [1], the compound of the general formula [1] may be produced according to the following scheme 2. According to the scheme 2, there can be produced a compound of the general formula [1] in which X¹, Y¹, L¹ to L⁴, R¹, R², R⁵ to R⁷, and R¹⁰ to R¹² are the same as X⁴, Y², L⁵ to L⁸, R⁴, R¹³ to R¹⁵, and R¹⁸ to R²⁰, respectively.

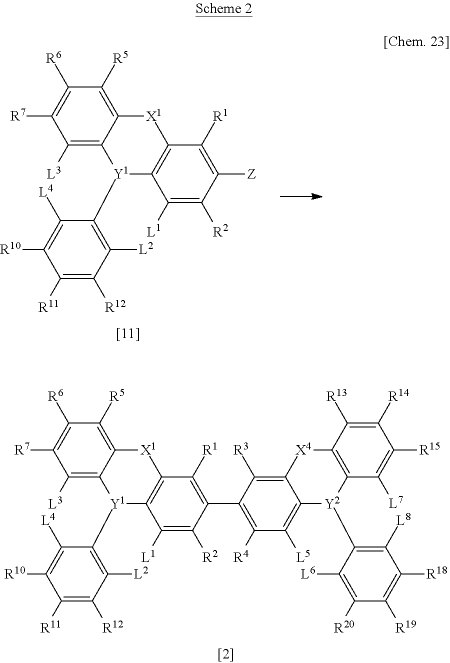

The reaction of the scheme 2 is a coupling reaction, for which, in general, a coupling agent is used. For example, the reaction can be attained in the presence of bis(1,5-cyclooctadiene)nickel [Ni(COD)₂], 2,2′-bipyridyl [bpy], or 1,5-cyclooctadiene [COD]. The coupling reaction itself using the reagent of the type has already been known, and the reaction condition of the scheme 2 can be optimized based on the known reaction conditions.

The reaction of the scheme 1 and the scheme 2 can be attained in a solvent that solves the compound of the general formula [11] and the compound of the general formula [12], and for example, the reaction may be carried out in tetrahydrofuran [THF]. The reaction temperature is not specifically defined, but preferably the reaction is carried out with heating at a temperature not higher than the boiling point of the solvent used. For example, when THF is used as the solvent, the reaction is carried out preferably at 40 to 66° C., more preferably at 55 to 66° C.

The production method of the scheme 1 is applicable also to production of a compound of the general formula [1] in which Ar¹ is not a single bond. For example, for producing a compound of the general formula [6] in which Ar¹ in the general formula [1] is a 1,3-phenylene group, a compound represented by the following general formula [13] may be used in place of the compound represented by the general formula [11] in the scheme 1. Other compounds of the general formula [1] may also be produced in the same manner.

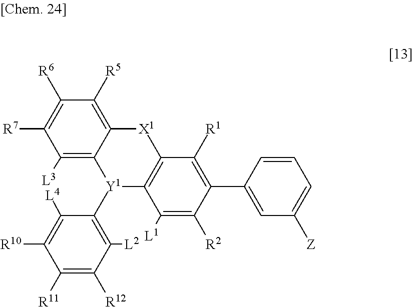

The compound of the general formulae [5] to [7] may also be produced by converting the compound of the above-mentioned general formula [11] into a dioxaborane form represented by the following general formula [14] followed by reacting it with 1,4-dibromobenzene, 1,3-dibromobenzene or 1,3,5-tribromobenzene. The dioxaborane form of the general formula [14] can be produced by reacting the compound of the general formula [11] with, for example, n-butyllithium followed by reacting it with 2-isopropoxy-4,4,5,5-tetramethyl-1,3,2-dioxaborolane. In converting the dioxaborane form into the compound of the general formulae [5] to [7], for example, it is desirable that the reaction is promoted with using tris(dibenzylideneacetone)palladium/chloroform abduct [Pd₂(dba)₃.CHCl₃] or 2-dicyclohexylphosphino-2′,6′-dimethoxybiphenyl [SPhos]. For the details of the reaction, Synthesis Examples to be given hereinunder are referred to.

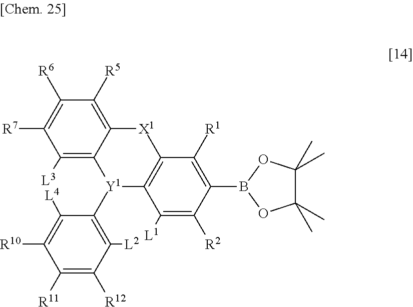

The compounds represented by the general formulae [11] and [12], which are the starting compounds in the scheme 1 and the scheme 2, and the compound represented by the above-mentioned general formula [13] can be produced, for example, according to the following scheme 3. In the scheme 3, described is a case of the production method for a compound of the general formula [11] where X¹ is —O—, L¹ and L² bond to each other to form —O— and Y¹ is >N—. Preferably, Z is a bromine atom.

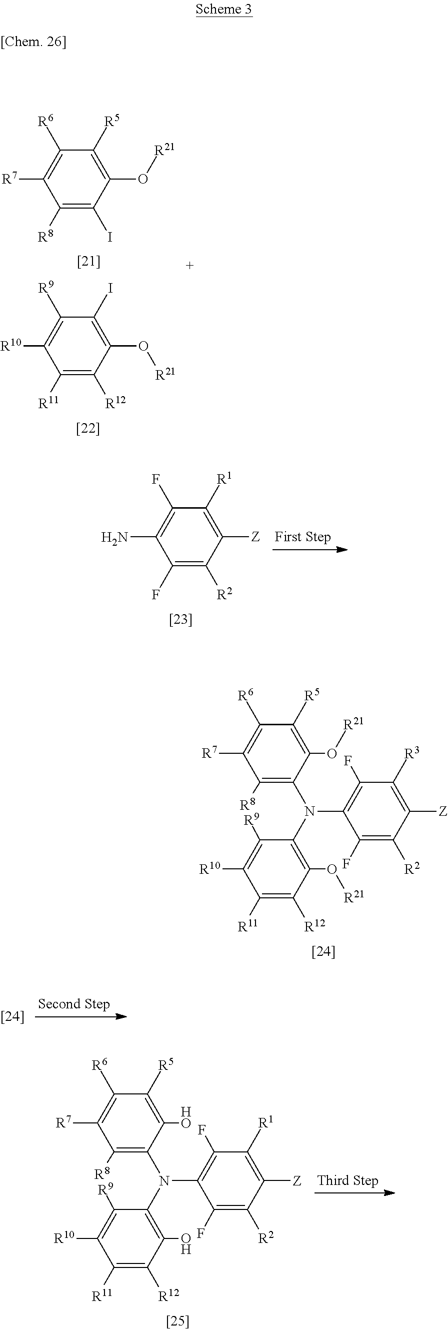

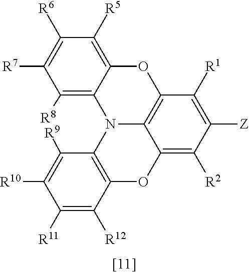

The definitions of R¹, R², R⁵ to R⁷ and R¹⁰ to R¹² in the general formulas [21] to [25] are the same as in the general formula [11]. R⁸ and R⁹ each independently represent a hydrogen atom or a substituent. In the general formulae [21], [22] and [24], R²¹ represents an alkyl group and is preferably an alkyl group having from 1 to 3 carbon atoms, more preferably a methyl group.

In the first step of the scheme 3, first, the compounds of the general formulae [21] and [22] that are o-alkoxyiodobenzenes are reacted with the compound of the general formula [23] that is a 2,6-difluoroaniline. In case where R⁵, R⁶, R⁷ and R⁸ in the general formula [24] which is to be produced in the first step are the same as R¹², R¹¹, R¹⁰ and R⁹, respectively, an o-alkoxyiodobenzene of the same type may be reacted with the compound of the general formula [23]. Preferably, the reaction is carried out in the environment in which the coupling reaction of the compounds of the general formulae [21] and [22] and the compound of the general formula [23] can be promoted. For example, Cu is preferably used in the presence of potassium carbonate or the like. The reaction condition using these reagents can be optimized with reference to similar coupling reaction conditions. The reaction of the first step may also be carried out in two stages as follows: First, one molecule of the compound of the general formula [21] is reacted by coupling with one molecule of the compound of the general formula [23], and then further reacted by coupling with one molecule of the compound of the general formula [22]. Selecting the catalyst to be used in the first coupling reaction makes it possible to prevent two molecules of the compound of the general formula [21] from being coupled with one molecule of the compound of the general formula [23]. As the catalyst, for example, CuI is usable.

The reaction of the first step may be carried out in a solvent that dissolves the compounds of the general formulae [21] to [23], and for example, the cation may be carried out in o-dichlorobenzene [ODCB]. The reaction temperature is not specifically defined, but preferably the reaction is carried out with heating at a temperature not lower than the boiling point of the solvent used. For example, when ODCB is used as the solvent, preferably, the reaction may be carried out at 150 to 180° C., more preferably under reflux at the boiling point of the solvent.

In the second step of the scheme 3, the alkoxy group of the compound represented by the general formula [24], as obtained in the first step, is converted into a hydroxyl group, thereby providing the compound represented by the general formula [25]. In the second step, known conditions of conversion reaction from alkoxy group to hydroxyl group may be combined suitably. For example, the compound is first reacted with boron tribromide in a methylene chloride solvent, and then reacted with hydrochloric acid. The product obtained in the second step may be used in the next third step, without being purified or isolated.

In the third step of the scheme 3, the hydroxyl group and the fluorine atom of the compound represented by the general formula [25], as obtained in the second step, are reacted in a mode of intramolecular cyclization to give the compound represented by the general formula [11]. The reaction may be promoted, for example, by heating in the presence of an alkali such as potassium carbonate or the like. Preferably, the heating temperature is from 70 to 130° C. or so. As the solvent, preferably used is dimethylformamide [DMF] or the like.

The compounds represented by the general formula [12] and the general formula [13] that are the starting compounds in the scheme 1 may also be produced according to the scheme 3. In addition, other similar compounds may also be produced in the same manner.

The production route of the scheme 3 is a novel production route and is advantageous in that, as compared with a heretofore-known production method for an oxygen-crosslinked triarylamine or a sulfur-crosslinked triarylamine (M. Kuratsu et. al., Chem. Lett., Vol. 33, No. 9 (2004)), the yield in the route is good and route facilitates mass-production. In addition, since both the compound of the general formula [21] and the compound of the general formula [22], each having a different structure, can be reacted by coupling with the compound of the general formula [23], the route has another advantage in that the compound in which the aryl groups to be crosslinked are asymmetric can be readily produced. Further, still another advantage of the route is that, when a bromide compound (for example, a compound where Z is a bromine atom) is used as the compound of the general formula [23], then a crosslinked triarylamine bromide can be produced, and it is easy to produce a compound having multiple main skeletons.

The production route of the scheme 3 may be generalized, for example, as the following production method.

(Production Method for 2,2′:6,2″-dioxatriphenylamine Compound)

A production method for a 2,2′:6,2″-dioxatriphenylamine compound, which comprises coupling one molecule of a 2,6-difluoroaniline compound and two molecules of a 2-alkoxyiodobenzene compound to prepare an N,N-bis(2-alkoxyphenyl)-2,6-difluoroaniline,

then converting the alkoxy group in the resulting N,N-bis(2-alkoxyphenyl)-2,6-difluoroaniline compound into a hydroxyl group to give an N,N-bis(2-alkoxyphenyl)-2,6-difluoroaniline compound, and further reacting the compound for intramolecular cyclization to give a 2,2′:6,2″-dioxatriphenylamine compound.

Previously introducing a substituent corresponding to Z into the benzene ring of the starting compound, 2,6-difluoroaniline compound or 2-alkoxyiodobenzene compound makes it possible to introduce Z into the corresponding benzene ring of the 2,2′:6,2″--dioxatriphenylamine compound to be obtained finally. Two molecules of the 2-alkoxyiodobenzene compound to be coupled may be the same or different two molecules. In case where different two molecules are used, preferably, the two molecules to be used differ in point of the substituent therein. In such a case, employable is successive coupling reaction of stepwise coupling the two molecules one by one. When Pd is used, one molecule alone can be coupled efficiently.

(Production Method for 2,2′:6,2″-dithiatriphenylamine Compound)

A production method for a 2,2′:6,2″-dithiatriphenylamine compound, which comprises coupling one molecule of a 2,6-difluoroaniline compound and two molecules of a 2-alkylthioiodobenzene compound to prepare an N,N-bis(2-alkylthiophenyl)-2,6-difluoroaniline,

then converting the alkylthio group in the resulting N,N-bis(2-alkylthiophenyl)-2,6-difluoroaniline compound into a thiol group to give an N,N-bis(2-mercaptophenyl)-2,6-difluoroaniline compound, and further reacting the compound for intramolecular cyclization to give a 2,2′:6,2″-dithiatriphenylamine compound.

Previously introducing a substituent corresponding to Z into the benzene ring of the starting compound, 2,6-difluoroaniline compound or 2-alkylthioiodobenzene compound makes it possible to introduce Z into the corresponding benzene ring of the 2,2′:6,2″-dioxatriphenylamine compound to be obtained finally. Two molecules of the 2-alkylthioiodobenzene compound to be coupled may be the same or may differ in point of the substituent therein. Regarding the successive coupling reaction in coupling the different two molecules, referred to is the description given above.

The compound represented by the general formula [1] and produced according to any of the schemes 1 to 3 or the like may be applied to specific use after purified and isolated, but in some use cases, the compound maybe used without being isolated. The invention also encompasses a composition containing both the compound represented by the general formula [1] and a compound not represented by the general formula [1]. In addition, the invention further encompasses a composition containing different types of the compound represented by the general formula [1]. The synthesized compound of the general formula [1] may be purified by suitably selecting known purification methods of column chromatography, etc.

[Physical Properties of Compound Represented by general Formula [1]]

The compound represented by the general formula [1] has a semi-planar structure and therefore multiple molecules thereof can be densely packed with preventing crystallization. Through computational chemistry, the present inventors have confirmed that the compound represented by the general formula [1] is a material having a small rearrangement energy and having a large intermolecular transfer integral. In addition, the compound represented by the general formula [1] has a sufficient molecular site. Having the above-mentioned characteristics, the compound represented by the general formula [1] have a high glass transition temperature and secures an amorphous state stably existing therein. Further, the orbital level of HOMO (highest occupied molecular orbital) and LUMO (lowest unoccupied molecular orbital) of the compound represented by the general formula [1] is on a level suitable as that for a charge transport material. In particular, the compound represented by the general formula [1] where Y¹ and Y² each are >N— or >P— exhibits properties useful as a hole transport material. The compound represented by the general formula [1] where Y¹ and Y² each are >B— or >P(═O)— exhibits properties useful as an electron transport material. The term, charge transport material as referred to in the invention has a concept that includes such a hole transport material and an electron transport material.

The compound represented by the general formula [1] is excellent as a charge transport material, and is therefore effectively used in various organic devices, especially in organic electronic devices. For example, the compound can be effectively used in organic electroluminescence elements and electrophotographic photoreceptors. In addition, since the compound can be effectively used in photoelectric conversion elements, the compound can also be effectively used in organic thin-film solar cells. Further, the compound can be effectively used in organic transistors. As typical organic devices, an organic electroluminescence element and an organic thin-film solar cell are described hereinunder.

### [Organic Electroluminescence Element]

A typical organic electroluminescence element is so configured that an anode of ITO or the like, a hole injection layer, a hole transport layer, a light-emitting layer, an electron transport layer, an electron injection layer and a cathode are laminated on a transparent substrate of glass or the like. The compound represented by the general formula [1] of the invention can be used as a material of those hole injection layer, hole transport layer, light-emitting layer, electron transport layer and electron injection layer, depending on the physical properties thereof. For example, when the compound of the general formula [1] (in particular, the compound where Y¹ and Y² each are >B— or >P(═O)˜) useful as an electron transport material is used in the electron transport layer, the electron that is injected from the cathode into the electron transport layer via the electron injection layer can be efficiently transported to the light-emitting layer. Accordingly, the efficiency in recombination of electron and hole in the light-emitting layer can be increased, and a high luminance efficiency can be realized with suppressing the consumption power and the heat generation amount. As a result, prolongation of the life of the organic electroluminescence element can be thereby realized. In another case, when the compound represented by the general formula [1] (in particular, the compound where Y¹ and Y² each are >N— or >P—) useful as a hole transport material is used: in the hole transport layer, the hole that is injected from the anode into the hole transport layer via the hole injection layer can be efficiently transported to the light-emitting layer. Accordingly, the efficiency in recombination of electron and hole in the light-emitting layer can be increased and a high luminance efficiency can be realized with suppressing the consumption power and the heat generation amount. As a result, prolongation of the life of the organic electroluminescence element can be thereby realized.

In the organic electroluminescence element using the compound of the invention, known materials used in organic electroluminescence elements can be suitably selected and combined. If desired, known techniques as well as various modifications that may be readily derived from known techniques may be given to the organic electroluminescence element using the compound of the invention.

### [Organic Thin-Film Solar Cell]

A typical organic thin-film solar cell is so configured that an anode of ITO or the like, a hole transport layer, a photoelectric conversion layer, an electron transport layer and a cathode are laminated on a transparent substrate of glass or the like. The photoelectric conversion layer has a p-type semiconductor layer on the anode side and has an n-type semiconductor layer on the cathode side. The compound represented by the general formula [1] of the invention can be used as a material of those hole transport layer, p-type semiconductor layer, n-type semiconductor layer and electron transport layer, depending on the physical properties thereof. The compound represented by the general, formula [1] of the invention can function as a hole transport material or an electron transport material in the organic thin-film solar cell. It is also possible to use the compound represented by the general formula [1] of the invention to produce a polymer containing the skeleton represented by the general formula [1] as the recurring unit therein, thereby using the polymer in the organic thin-film solar cell.

The organic thin-film solar cell using the compound of the invention may be optionally provided with a hole block layer, an electron block layer, an electron injection layer, a hole injection layer, a planarization layer and the like, in addition to the above. In the organic thin-film, solar cell using the compound of the invention, known materials used in organic thin-film solar cells can be suitably selected and combined. If desired, known techniques as well as various modifications that may be readily derived from known techniques may be given to the organic thin-film solar cell using the compound of the invention.

### EXAMPLES

The characteristics of the invention are described more concretely with reference to the following Examples. In the following Examples, the materials used, the details of the treatment and the treatment process may be suitably modified or changed not overstepping the spirit and the scope of the invention. Accordingly, the invention should not be limitatively interpreted by the Examples mentioned below.

### Example 1

Compound 1 was produced according to the following scheme.

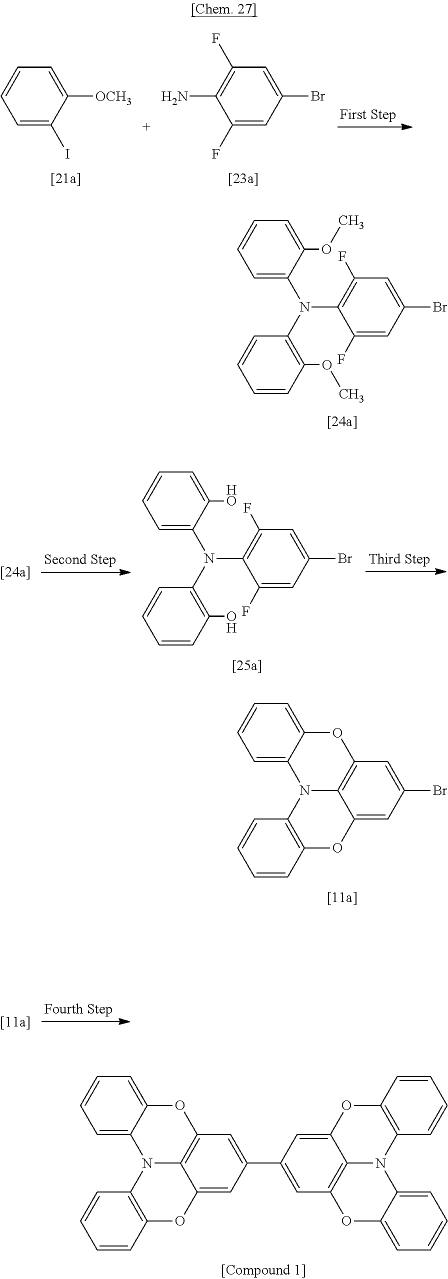 [SMILES: COc1ccccc1I ; Nc1c(F)cc(Br)cc1F ; COc1ccccc1N(c1ccccc1OC)c1c(F)cc(Br)cc1F ; Oc1ccccc1N(c1ccccc1O)c1c(F)cc(Br)cc1F ; Brc1cc2c3c(c1)Oc1ccccc1N3c1ccccc1O2 ; c1ccc2c(c1)Oc1cc(-c3cc4c5c(c3)Oc3ccccc3N5c3ccccc3O4)cc3c1N2c1ccccc1O3]

Compound 21a (19.8 g, 84.6 mmol), compound 23a (7.74 g, 37.2 mmol), K₂CO₃ (21.5 g, 156 mmol), and Cu (7.82 g, 123 mmol) were dissolved in o-dichlorobenzene [ODCB] (100 ml) and heated at 180° C. for 110 hours. The reaction mixture was filtered, and the insoluble matter was washed three times with chloroform (100 ml). The filtrate was washed with water, then dried with MgSO₄, filtered and concentrated under reduced pressure. Further, the obtained black solid was washed with hexane to give a white powder, compound 24a (10.6 g, 20.4 mmol) at a yield of 68%. Mp: 157.5-153.5° C.

¹H NMR (300 MHz, CDCl₃, ppm) δ7.10-6.34 (m, 4H), 6.94-6.81 (m, 6H), 3.60 (s, 6H).
¹³C NMR (75 MHz, CDCl₃, ppm): δ158.24 (dd, ¹J (C, F)=252.4, ³J (C, F)=6.9 Hz), 153.25, 136.12, 124.61, 121.10, 115.29 (dd, ²J (C, F)=17.8, ⁴J (C, F)=9.2 Hz), 114.70 (t, ³J (C, F)=12.0 Hz), 113.00, 56.03.
HRMS (FAB): m/z 419.0325 (M⁺); calcd for C₂₀H₁₆BrF₂NO: 419.0332.
Anal. calcd (%) for C₂₀H₁₆BrF₂NO₂: C57.16, H3.84, N3.33, found: C57.15, H3.90, N3.40.

A dewatered CH₂Cl₂ (450 ml; solution of the compound 24a (9.75 g, 23.3 mmol) was cooled to −78° C., and BBR₃ (4.50 ml, 47.5 mmol) was added thereto and thereafter gradually heated up to room temperature and then stirred for 3 hours. Subsequently, the solution was added to water (100 ml), and extracted three times with CH₂Cl₂ (100 ml). The obtained organic layer was dried with Na₂SO₄, filtered and concentrated under reduced pressure to give a white powder, compound 25a (8.38 g, 21.4 mmol) at a yield of 92%.

### ¹H NMR (300 MHz, CDCl₃) δ7.12-7.03 (m, 6H), 6.87 (td, ³J (H, H)=7.8 Hz, ⁴J (H, H)=1.5 Hz, 4H).

The compound 25a (6.68 g, 17.1 mmol) and anhydrous K₂CO₃ (7.15 g, 51.8 mmol) were put into dimethylformamide [DMF] (150 ml), heated up to 100° C. and kept heated for 22 hours. Restoring to room temperature gave a white precipitate. The precipitated solid was taken out through filtration, washed with water and then dried under reduced pressure to give a white crystal, compound 11a (4.73 g, 13.5 mmol) at a yield of 79% . The filtrate was concentrated under reduced pressure and purified through silica gel column chromatography (Rf=0.78) using CH₂Cl₂ as a developing solvent, thereby giving a white crystal, compound 11a (0.931 g, 2.65 mmol). The two were combined to be the compound 11a (5.66 g, 16.1 mmol) at a yield of 94%.

### Mp: 215.5-216.3° C.

¹H NMR (300 MHz, CDCl₃, ppm) ε7.31 (dd, ³J (H, H)=7.5, ⁴J (H, H)=1.8 Hz, 2H), 6.99-6.85 (m, 6H), 6.66 (s, 2H).
¹³C NMR (75 MHz, CDCl₃, ppm) δ146.67, 145.72, 128.70, 123.91, 123.63, 120.39, 117.53, 115.21, 114.68, 114.48.
HRMS (FAB): m/z 350.9895 (M⁺), calcd for C₁₈H₁₀BrNO₂: 350.9908.
Anal. calcd (%): calcd for C₁₈H₁₀BrNO₂: C61.39, H2.86, N3.98; found: C61.01, H3.00, N4.02.

The compound 11a (4.20 g, 12.0 mmol), bis(1,5-cyclooctadiene)nickel [Ni(cod)₂] (3.96 g, 14.4 mmol), 1,5-cyclooctadiene [COD] (1.77 g, 16.4 mmol), and 2,2′-bipyridyl [bpy] (2.25 g, 14.4 mmol) were dissolved in tetrahydrofuran [THF] (360 ml) and heated at 60° C. for 24 hours. The mixture was dissolved in carbon disulfide and adsorbed by silica gel, and extracted with carbon disulfide (1000 ml) serving as a developing solvent. The solvent, was evaporated away from the obtained solution under reduced pressure to give a yellow solid, compound 1 (1.60 g, 2.94 mmol) at a yield of 49%. The silica gel that had been processed for extraction with carbon disulfide was further extracted using a Soxhlet extractor to give a yellow solid, compound 1 (0.510 g, 0.940 mmol). The two were combined to be the compound 1 (2.11 g, 3.88 mmol) at a yield of 65%. Further, the obtained compound 1 was purified through sublimation (320° C. to 350°0 C., 1 mmHg) to give a yellow crystal for use for various measurements,

### Mp: 375.2-376.1° C.

¹H NMR (300 MHz, CD₂Cl₂, ppm) δ7.36 (d, ³J (H, H)=6.9 Hz, 4H), 7.05-6.90 (m, 12H), 6.69 (s, 4H).
¹³C NMR (150 MHz, CD₂Cl₂/CS₂, ppm): δ147.15, 145.76, 136.04, 129.12, 124.16, 124.03, 120.50, 117.93, 115.00, 109.47.
HRMS (FAB): m/z 544.1429 (M⁺); calcd for C₃₆H₂₀N₂O₄: 544.1423.
Anal. calcd (%): calcd for C₃₆H₂₀N₂O₄: C79.40, H3.70, N5.14; found: C79.57, H3.88, N5.13.

### Example 2

Compound 2 was produced according to the following scheme.

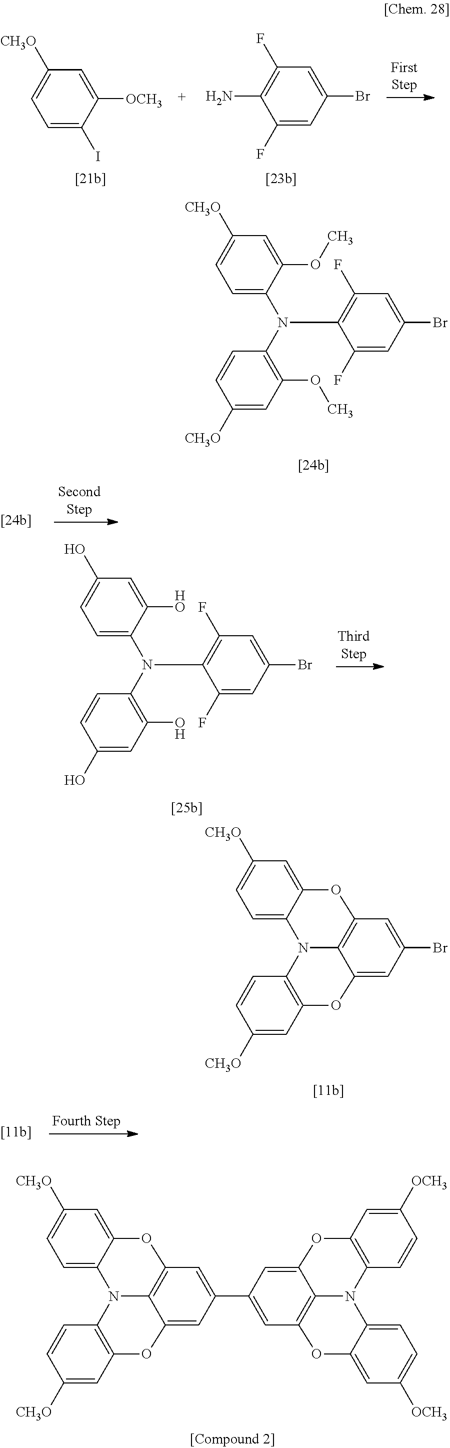

Compound 21b (20.4 g, 77.2 compound 23b (6.86 g, 33.0 mmol ), K₂CO₃ (18.2 g, 1.32 mmol), and Cu (6.80 g, 107 mmol) were dissolved in o-dichlorobenzene [ODCB] (90 ml) and heated at 180° C. for 150 hours. The insoluble matter was removed through filtration, washed three tunes with CH₂Cl₂ (100 ml), and the filtrate was wasted with water. The obtained organic phase was dried with MgSO₄, filtered, and then concentrated under reduced pressure. Further, this was purified through silica gel column chromatography (developing solvent:hexane/CH₂Cl₂ (⅓), Rf=0.56) to give a white solid, compound 24b (9.63 g, 20.1 mmol) at a yield of 61%.

### Mp: 96.4-97.3° C.

¹H NMR (300 MHz, CDCl₃, ppm) δ7.05-6.90 (m, 2H), 6.83 (d, ³J (H, H)=8.4 Hz, 2H), 6.46 (d, ⁴J (H, H)=2.7 Hz, 2H), 6.38 (dd, ³J (H, H)=8.7, ⁴J (H, H)=2.7 Hz, 2H), 3.78 (s, 6H), 3.60 (s, 6H).
¹³C NMR (75 MHz, CDCl₃, ppm) δ158.16 (dd, ¹J (C, F)=251.9, ³J (C, F)=7.4 Hz), 156.98, 154.34, 130.03, 125.30, 115.21 (dd, ²J (C, F)=17.8, ⁴J (C, F)=9.2 Hz), 113.51 (t, ³J (C, F)=12.1 Hz), 104.49, 100.30, 56.00, 55.34.
HEMS (FAB): m/z 479.0544 (M⁺); calcd for C₂₂H₂₀F₂NO₂: 479.0544.
Anal. calcd (%): calcd for C₂₂H₂₀BrF₂NO₂: C55.01, H4.20, N2.92; found; C54.99, H4.18, N2.99.

A CH₂Cl₂ (190 ml) solution of the compound 24b (4.68 g, 9.77 mmol) was cooled to −78° C., BBr₃ (10.0 g, 40.1 mmol) was added thereto, and then gradually heated up to room temperature and stirred overnight. Subsequently, 1.0M hydrochloric acid (100 ml) was added thereto, and extracted three times with ethyl acetate (50 ml). The obtained organic layer was dried with Na₂SO₄, filtered, and concentrated under reduced pressure to give a bluish black solid, compound 25b (4.08 g, 9.61 mmol) at a yield of 98%.

¹H NMR (300 MHz, CDCl₃, ppm) δ7.05-7.00 (m, 2H), 6.98 (d, ³J (H, H)=9.0 Hz, 2H), 6.39 (d, ⁴J (H, H)=2.7 Hz, 2H), 6.33 (dd, ³J (H, H)=9.0 Hz, ⁴J (H, H)=3.0 Hz, 2H), 5.57 (br, 2H), 4.70 (br, 2H).

The compound 25b (3.50 g, 8.26 mmol) and anhydrous K₂CO₃ (6.85 g, 49.6 mmol) were dissolved in dimethylformamide [DMF] (200 ml), and heated up to 100° C. and stirred for 14 hours. Subsequently, CH₃I (2.00 ml, 32.1 mmol) was added to the mixture and heated at 60° C. for 3 hours. The mixture was put into 1 M hydrochloric acid (200 ml), and then the aqueous layer was extracted three times with ethyl acetate (100 ml), dried with Na₂SO₄ and concentrated under reduced pressure. Further, this was purified through silica gel column chromatography (RF=0.85) using CH₂Cl₂ as the developing solvent to give a white solid, compound 11b (2.29 g, 5.56 mmol) at a yield of 67%.

### Mp: 175.5-177.4° C.

¹H NMR (300 MHz, C₆D₆, ppm) δ6.89 (d, ³J (H, H)=9.7 Hz, 2H), 6.56 (s, 2H), 6.46 (d, ⁴H (H, H)=2.7 Hz, 2H), 6.28 (dd, ³J (H, H)=9.7 Hz, ⁴J (H, H)=2.7 Hz, 2H), 3.21 ppm (2, 6H; OMe).
¹³C NMR (75 MHz, CDCl₃, ppm) δ156.73, 148.10, 146.03, 122.90, 121.63, 115.38, 115.13, 109.27, 104.61, 55.34 ppm.
HRMS (FAB): m/z 411.0087 (M⁺); calcd for C₂₀H₁₄BrNO₄: 411.0106.
Anal. calcd (%): calcd for C₂₀H₁₄BrNO₄: C58.27, H3.42, N3.40; found; C58.35, H3.44, H3.39.

The compound 11b (1.85 g, 4.50 mmol), bis(1,5-cyclooctadiene)nickel [Ni(cod)₂] (1.49 g, 5.41 mmol), 1,5-cyclooctadiene [COD] (0.586 g, 5.42 mmol), and 2,2′-bipyridyl [bpy] (0.843 g, 5.40 mmol) were dissolved in tetrahydrofuran [THF] (130 ml) and heated at 60° C. for 12 hours. The mixture solution was concentrated under reduced pressure, then toluene (100 ml) was added thereto, the resulting mixture was adsorbed by silica gel, extracted with toluene using a Soxhlet extractor, concentrated under reduced pressure and filtered with hexane to give a yellow solid, compound 2 (0.810 g, 1.22 mmol) at a yield of 54%. Further, this was purified through sublimation (285 to 310° C., 0.06 to 0.08 mmHg) and used for various measurements.

Mp (decomposition temperature): 351.8-353.8° C.
¹H NMR (300 MHz, CD₂Cl₂, ppm) δ6.99 (d, ³J (H, H)=8.7 Hz, 4H), 6.44 (s, 4H), 6.34 (d, ⁴J (H, H)=2.7 Hz, 4H0, 6.27 (dd, ³J (H, H)=8.7, ⁴J (H, H)=2.7 Hz, 4H), 3.52 ppm (s, 12H).
HRMS (FAB): m/z 664.1818 (M⁺); calcd for C₄₀H₂₈N₂O₈: 664.1846.
Anal. calcd (%): calcd for C₄₀H₂₈N₂O₈: C72.28, H4.25, N4.21; found: C72.33, H4.28, N4.25.

### Example 3

Compound 24 was produced according to the following scheme.

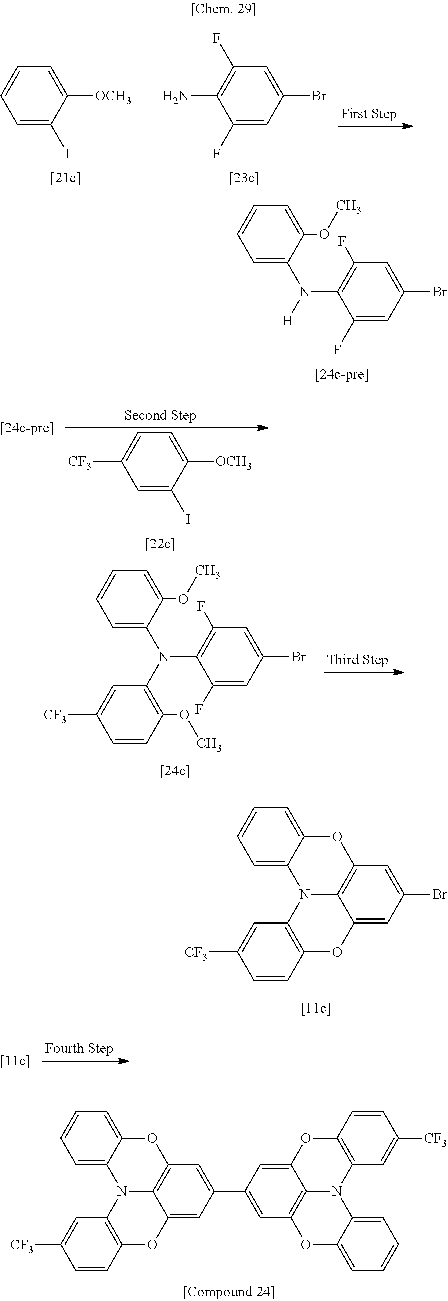

Compound 21c (11.7 g, 49.9 mmol), compound 23c (9.19 g, 44.2 mmol), Pd₂(dba)₃.CHCl₃ (0.799 g, 0.765 mmol), sodium tert-butoxide (4.38 g, 45.6 mmol), and tri-tert-butylphosphine (0.920 g, 4.55 mmol) were dissolved in dry toluene 9100 ml), and stirred at 100° C. for 26 hours. The insoluble matter was filtered, and washed with toluene (60 ml). Subsequently, water was added to the filtrate, and extracted with toluene (50 ml×3). The organic layer was dried with Na₂SO₄, filtered, and the filtrate was concentrated under reduced pressure. The obtained solid was purified through silica gel column chromatography (CH₂Cl₂/hexane=1/5, R_{f}=0.30) to give a white solid, compound 24c-pre (7.73 g, 24.6 mmol) at a yield of 50%.

### Mp: 71.2-72.2° C.

¹H NMR (300 MHz, CDCl₃, ppm) δ7.20-7.11 (m, 2H), 6.91-6.80 ppm (m, 3H), 6.57 (td, ³J (H, H)=8.7 Hz, ⁴J (H, H)=2.7 Hz, 1H), 5.83 (s, 1H), 3.93 (s, 3H).
¹³C NMR (75 MHz, CDCl₃, ppm) δ156.67 (dd, ¹J (C, F)=250.1 Hz, ³J (C, F)=6.3 Hz), 147.74, 132.54, 120.70, 120.24, 118.67 (t, ³J (C, F)=14.9 Hz), 115.79 (dd, ²J (C, F)=18.3 Hz, ⁴J (C, F)=8.6 Hz), 114.62 (t, ³J (C, F)=11.7 Hz), 113.17 (t, ⁴J (C, F)=2.9 Hz), 110.11, 55.58 ppm.
HRMS (FAB): m/z 312.9923 (M⁺), calcd for C₁₃H₁₀BrF₂NO: 312.9914.
Anal. calcd (%): calcd for C₁₃H₁₀BrF₂NO: C49.71, H3.21, N4.46; found: C49.79, H3.17, N4.52.

2-Methoxy-5-trifluoromethylaniline (10.1 g, 53.1 mmol) was dissolved in acetonitrile (160 ml), and an aqueous solution of 12 M HCl (11.0 ml) was added thereto and cooled to 0° C. Sodium nitrile (4.76 g, 71.0 mmol) dissolved in 30 ml of water was dropwise added to the solution, taking 10 minutes, and there stirred for 20 minutes. Further, potassium iodide (26.6 g, 160 mmol) dissolved in 60 ml of water was dropwise added thereto, taking 15 minutes, then stirred for 2 hours, restored to room temperature, and further stirred for 20 hours. An aqueous Na₂SO₃ solution (50 ml) was added thereto, and extracted with ether (60 ml×3). The organic layer was dried with Na₂SO₄, filtered, and the filtrate was concentrated under reduced pressure. the obtained oil was purified through silica gel short column chromatography (hexane, R_{f}=0.40) to give a colorless oil, compound 22c (14.8 g, 49.0 mmol) at a yield of 92%.

¹H NMR (300 MHz, CDCl₃, ppm) δ8.01 (d, ⁴J (H, H)=2.1 Hz, 1H), 7.59 (dd, ³J (H, H)=9.0 Hz, ⁴J (H, H)=2.1 Hz, 1H), 6.51 (d, ³J (H, H)=9.0 Hz, 1H), 3.94 (s, 3H).

The compound 24c-pre (0.862 g, 2.75 mmol), the compound 22c (0.990 g, 3.28 mmol), K₂CO₃ (0.857 g, 6.20 mmol), and copper powder (0.317 g, 4.99 mmol) were added to dry ODCB (20 ml) heated up to 180° C., and stirred for 65 hours. The insoluble matter was filtered and washed with dry CH₂Cl₂ (50 ml). Subsequently, water (20 ml) was added to the filtrate, and extracted with CH₂Cl₂ (10 ml×3). The organic layer was dried with Na₂SO₄, filtered, and the filtrate was concentrated under reduced pressure. The obtained solid was purified through silica gel column chromatography (CH₂Cl₂/hexane=1/3, R_{f}=0.31) to give a white solid, compound 24c (1.00 g, 2.05 mmol) at a yield of 75%.

### mp: 98.4-99.4° C.

¹H NMR (300 MHz, CDCl₃, ppm) δ7.29 (d, ³J (H, H)=9.0 Hz, 1H), 7.12 (ddd, ³J (H, H)=7.2 Hz, ³J (H, H)=6.9 Hz, ⁴J (H, H)=2.1 Hz, 1H), 7.07 (d, ⁴J (H, H)=1.8 Hz, 1H), 7.02 (d, ³J (H, H)=8.1 Hz, 2H), 6.95-6.88 (m, 4H), 3.64 (s, 3H), 3.59 (s, 3H).
¹³C NMR (75 MHz, CDCl₃, ppm) δ158.49 (dd, ¹J (C, f)=2.5 Hz, ³J (C, F)=6.9 Hz), 155.04, 153.44, 136.35, 135.07, 125.60, 125.11, 124.18 (q, ¹J (C, F)=270 Hz), 124.16, 123.99, 123.17 (q, ²J (C, F)=32.6 Hz), 121.25, 120.98, 120.54, 120.49, 115.79, 115.477, 115.474 (dd, ²J (C, F)=18.1, ⁴J (C, F)=8.9 Hz), 113.00, 112.05, 56.03, 55.89 ppm.
HRMS (FAB): m/z 487.0206 (M⁺), calcd for C₂₁H₁₅BrF₅NO₂: 487.0206.
Anal. calcd (%): calcd for C₂₁H₁₅BrF₅NO₂: C51.66, H3.10, N2.87; found: C51.89, H3.09, N2.92.

The compound 24c (3.113 g, 6.38 mmol) was dissolved in dry CH₂Cl₂ (200 mL) and cooled to −78° C. BBr₃ (1.25 ml, 13.20 mmol) was added thereto, then gradually heated up to room temperature, and stirred for 3 hours. The solution was put in water (100 ml), and extracted with CH₂Cl₂ (50 ml×3). This was dried with Na₂SO₄, filtered, and then the filtrate was concentrated under reduced pressure to give 3.063 g of a solid (compound 25c) containing CH₂Cl₂. The obtained solid was dissolved in DMF (130 ml), then K₂CO₃ (2.642 g, 19.1 mmol) was added thereto, and stirred at 100° C. for 12 hours. An aqueous 1 M NH₄Cl solution (100 ml) was added to the reaction mixture, and the aqueous layer was extracted, with CH₂Cl₂ (80 ml×3). The organic layer was dried with Na₂SO₄, filtered and the filtrate was concentrated under reduced pressure. The obtained solid was processed for placing point removal in silica gel chromatography (CH₂Cl₂) and then purified through silica gel chromatography (CH₂Cl₂/hexane=1/5, R_{f}=0.66) to give a white solid, compound 11c (1.741 g, 4.14 mmol) at a yield of 65%.

### Mp: 146.1-147.0° C.

¹H NMR (300 MHz, CDCl₃, ppm) δ7.53 (d, 1H), 7.28 (dd, ⁴J (H, H)=2.1 Hz, ³J (H, H)=6.6 Hz, ⁴J (H, H)=1.2 Hz, 1H), 7.16 (d, ³J (H, H)=8.4 Hz, 1H), 7.04-6.88 (m, 4H), 6.74 (d, ³J (H, H)=8.4 Hz, 1H), 6.69 ppm (dd, ³J (H, H)=7.8 Hz, ⁴J (H, H)=2.1 Hz, 2H).
¹³C NMR (75 MHz, CDCl₃, ppm) δ149.22, 146.64, 145.63, 145.19, 129.52, 127.83, 126.50 (q, ²J (C, F)=33.2 Hz), 124.48, 123.67 (q, ¹J (C, F)=270 Hz), 120.67 (q, ³J (C, F)=4.0 Hz), 119.71, 117.92, 117.72, 115.89, 115.18, 114.62, 114.42, 111.59 ppm (q, ³J (C, F)=4.1 Hz).
HRMS (FAB): m/z 418.9783 (M⁺); calcd for C₁₉H₉BrF₃NO₂: 418.9769.
Anal. calcd (%): calcd for C₁₉H₉BrF₃NO₂: C54.31, H2.16, N3.33; found: C54.43, H2.42, N3.53.

The compound 11c (0.964 g, 2.29 mmol), Ni(cod)₂ (0.379 g, 1.38 mmol), 1,5-cyclooctadiene (0.35 ml, 2.85 mmol) and 2,2′-bipyridyl (0.432 g, 2.77 mmol) were dissolved in dry THF (60 ml), and heated at 60° C. for 14.5 hours. the solution was concentrated under reduced pressure, adsorbed by silica gel using toluene, and extracted with toluene using a Soxhlet extractor (R_{f}=0.96), and then concentrated under reduced pressure. The solid was washed with hexane to give a yellow solid, compound 24 (553.3 mg, 0.813 mmol) at a yield of 71%.

### Mp: 363.2-364.2° C.

¹H NMR (300 MHz, CD₂Cl₂, ppm) δ7.60 (⁴J (H, H)=1.2 Hz, 2H), 7.34 (dd, ³J (H, H)=7.8 Hz, ⁴J (H, H)=2.1 Hz, 2H), 7.20 (d, ³J (H, H)=9.0 Hz, 2H), 7.07-6.95 (m, 8H), 6.73 ppm (dd, ³J (H, H)=6.3 Hz, ⁴J (H, H)=1.8 Hz, 2H).
HRMS (FAB): m/z 680.1169 (M⁺), calcd for C₃₈H₁₈F₆N₂O₄: 680.1171.
Anal. calcd (%): calcd for C₃₈H₁₈F₆N₂O₄: C67.06, H2.67, N4.12; found: C67.20, H2.61, N4.25.

### Example 4

Compound 201 was produced according to the following scheme.

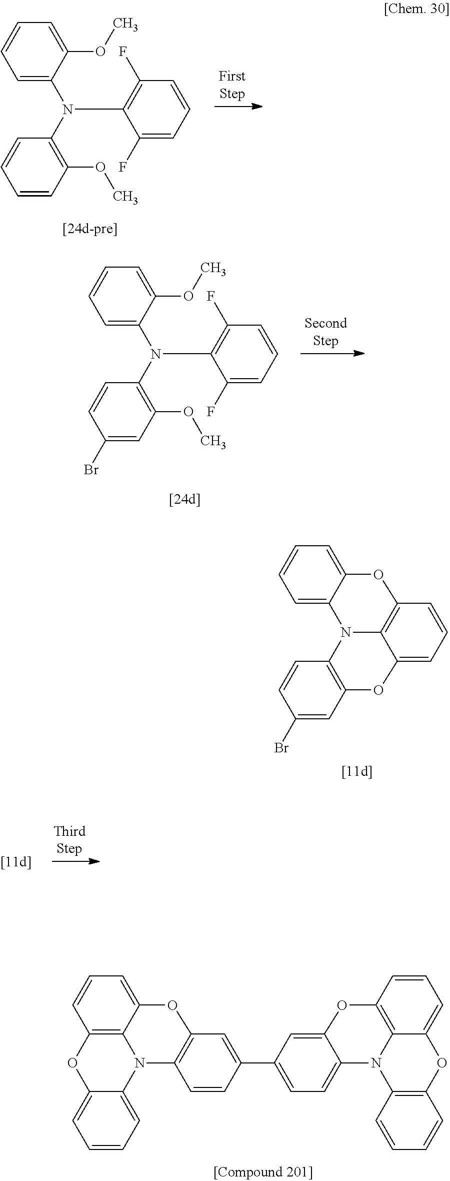 [SMILES: COc1ccccc1N(c1ccccc1OC)c1c(F)cccc1F ; COc1ccccc1N(c1ccc(Br)cc1OC)c1c(F)cccc1F ; Brc1ccc2c(c1)Oc1cccc3c1N2c1ccccc1O3 ; c1ccc2c(c1)Oc1cccc3c1N2c1ccc(-c2ccc4c(c2)Oc2cccc5c2N4c2ccccc2O5)cc1O3]

Compound 24d-pre (8.34 g, 24.5 mmol) and N-bromosuccinimide (4.35 g, 24.4 mmol) were dissolved in CHCl₃ (200 ml) and acetic acid (200 ml), and stirred at room temperature for 18 hours. This was neutralized with an aqueous saturated solution of NaHCO₃, and extracted with CHCl₃ (100 ml×3). The organic layer was dried with Na₂SO₄, filtered, and the filtrate was concentrated under reduced pressure. The obtained solid was purified through silica gel column chromatography (CH₂Cl₂/hexane=1/2, R_{f}0.45) to give a white solid, compound 24d (8.11 q, 19.3 mmol) at a yield of 79%.

### Mp: 119.1-120.1° C.

¹H NMR (300 MHz, CDCl₃, ppm) δ7.11-6.97 (m, 3H), 6.95 (dd, ³J (H, H)=8.4 Hz, ⁴J (H, H)=2.1 Hz, 1H), 6.93-6.76 (m, 5H), 6.74 (d, ³J (H, H)=8.4 Hz, 1H), 3.59 (s, 3H), 3.56 ppm (s, 3H).
¹³C NMR (75 MHz, CDCl₃ppm) δ158.96 (dd, ¹J (C, F)=249.5 Hz, ³J (C, F)=5.7 Hz), 153.46, 153.28, 136.06, 124.96, 124.77, 124.67, 124.40 (t, ³J (C, F)=6.9 Hz), 124.26, 123.13, 123.92, 121.13, 116.28, 116.05, 113.15, 111.54 (dd, ²J (C, F)=16.0 Hz, ⁴J (C, F)=6.8 Hz), 56.24, 56.05 ppm.
HRMS (FAB): m/z 419.0332 (M⁺), calcd for C₂₀H₁₆BrF₂NO₂: 419.0332.
Anal. calcd (%): calcd for C₂₀H₁₆BrF₂NO₂: C56.17, H3.84, N3.33; found: C56.26, H3.88, N3.38.

The compound 24d (1.82 g, 4.33 mmol) was dissolved in dry CH₂Cl₂ (90 ml). The solution was cooled to ˜78° C., then DBBr₃ (1.00 ml, 10.6 mmol) was added thereto and gradually heated up to room temperature, and stirred for 4 hours. The solution was put in water, and the aqueous layer was extracted with CH₂Cl₂ (50 ml×3). This was dried with Na₂SO₄, filtered, and the filtrate was concentrated under reduced pressure to give 1.74 g a white solid (compound 25d) containing CH₂Cl₂. The obtained solid was dissolved in DMF (60 ml), then K₂CO₃ (1.84 g, 13.3 mmol) was added thereto, and stirred at 100° C. for 15.5 hours. The solution was concentrated under reduced pressure, then water was added thereto, and extracted with CH₂Cl₂ (50 ml×3). The organic layer was dried with Na₂SO₄, filtered, and the filtrate was concentrated under reduced pressure. The obtained solid was purified through silica gel column chromatography (CH₂Cl₂, R_{f}=0.95) to give a white solid, compound 11d (1.51 g, 4.28 mmol) at a yield of 99%.

### Mp: 145.3-146.3° C.

¹H NMR (300 MHz, CDCl₃, ppm) δ7.25 (d, ³J (H, H)=6.9 Hz, 1H), 7.19 (dd, ³J (H, H)=6.9 Hz, ⁴J (H, H)=2.4 Hz, 1H), 7.07-7.02 (m, 2H), 6.98-6.8 (m, 3H), 6.6 (t, ³J (H, H)=8.4 Hz, 1H), 6.51 (dd, ³J (H, H)=8.4 Hz, ⁴J (H, H)=1.2 Hz, 1H), 6.49 ppm (dd, ³J (H, H)=7.5 Hz, ⁴J (H, H)=1.2 Hz, 1H).
¹³C NMR (75 MHz, CDCl₃, ppm) δ147.63, 146.88, 145.25, 144.93, 128.57, 128.44, 126.34, 123.88, 123.73, 123.65, 120.58, 117.58, 115.47, 114.47, 111.46, 111.13 ppm.
HRMS (FAB): m/z 350.9897 (M⁺), calcd for C₁₈H₁₀BrNO₂: 350.9895.
Anal. calcd (%): calcd for C₁₈H₁₀BrNO₂: C61.39, H2.86, N3.98; found: C61.53, H2.79, N4.00.

The compound 11d (0.351 g, 1.00 mmol), Ni(cod)₂ (0.329 g, 1.20 mmol), 1,5-cyclooctadiene (0.14 ml, 1.14 mmol) and 2,2′-bipyridyl (0.189 g, 1.21 mmol) were dissolved in dry THF (30 ml), and heated at 60° C. for 18 hours. The solution was concentrated under reduced pressure, adsorbed by silica gel using toluene, and extracted with toluene using a Soxhlet extractor (R_{f}=0.95), and then concentrated under reduced pressure. The solid was washed with hexane to give a yellow solid, compound 201 (0.268 g, 0.491 mmol) at a yield of 98%.

### Mp: 337.6-338.6° C.

¹H NMR (300 MHz, 1/1CD₂Cl₂/CS₂, ppm) δ7.38 (d, ³J (H, H)=8.4 Hz, 2H), 7.36 (dd, ³J (H, H)=8.1 Hz, ⁴J (H, H)=1.5 Hz, 2H), 7.16 (dd, ³J (H, H)=8.4 Hz, ⁴J (H, H)=2.1 Hz, 2H), 7.10 (d ⁴J (H, H)=2.1 Hz, 2H), 7.02-6.88 (m, 6H), 6.79 (t, ³J (H, H)=8.1 Hz, 2H), 6.53 (dd, ³J (H, H)=8.4 Hz, ⁴J (H, H)=1.2 Hz, 2H), 6.51 ppm (dd, ³J (H, H)=8.4 Hz, ⁴J (H, H)=1.2 Hz, 2H).
HRMS (FAB): m/z 544.1426 (M⁺), calcd. for C₃₆H₂₀N₂O₄:544.1423.
HRMS (FAB): m/z 544.1426 (M⁺), calcd for C₃₆H₂₀₂O₄: 544.1423.
Anal. calcd (%): calcd for C₃₆H₂₀N₂O₄: C79.40, H3.70, N5.14; found: C79.22, H3.59, N5.17.

### Example 5

Compound 285 was produced according to the following scheme.

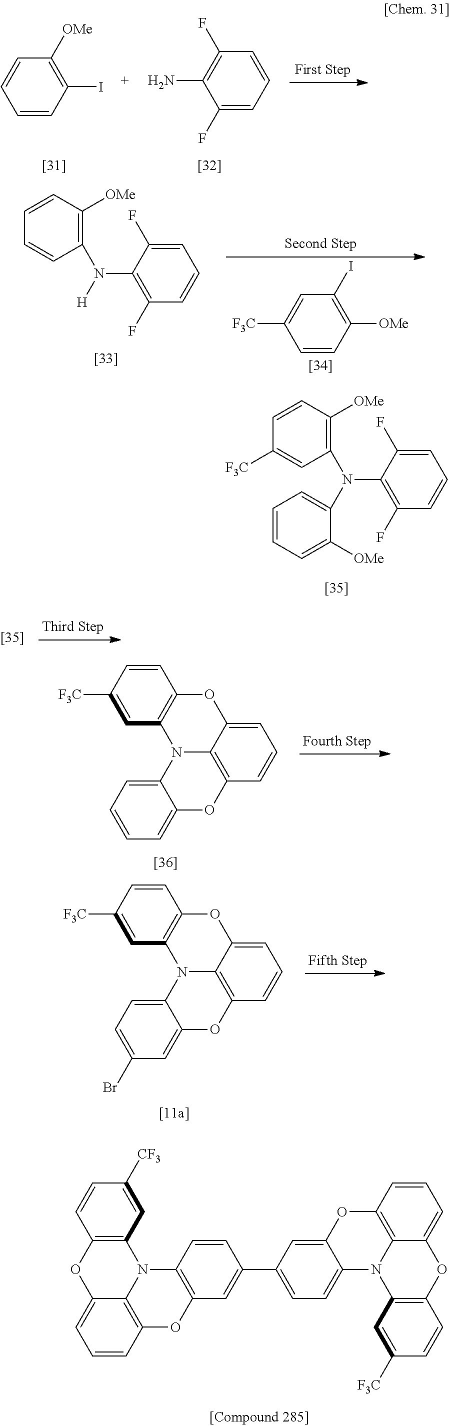

Compound 31 (21.7 g, 92.5 mmol), compound 32 (10.7 g, 82.7 mmol), Pd₂(dba)₃.CHCl₃ (1.60 g, 1.59 mmol), sodium tert-butoxide (9.22 g, 95.9 mmol), and tri-tert-butyl phosphine (2.58 g, 12.7 mmol) were dissolved in dry toluene (200 ml), and stirred at 100° C. for 16 hours. The insoluble matter was filtered, and washed with toluene (150 ml). Subsequently, water (50 ml) was added to the filtrate, and extracted with toluene (50 ml×3). The organic layer was dried with Na₂SO₄, filtered, and the filtrate was concentrated under reduced pressure. The obtained solid was processed through silica gel short-column chromatography (CH₂Cl₂/hexane=1/2, Rf=0.45), and then purified through silica gel short-column chromatography (CH₂Cl₂/hexane=1/4, Rf=0.25) to give an orange liquid, compound 33 (18.7 g, 79.4 mmol) at a yield of 96%.

¹H NMR (300 MHz, CDCl₃, ppm) δ7.10-6.93 (m, 3H), 6.92-6.80 (m, 3H), 6.60 (m, 1H), 5.88 (s, 1H), 3.93 (s, 3H).
¹³C NMR (75 MHz, CDCl₃, ppm) δ157.05 (dd, ¹J (C, F)=246.1 Hz, ³J (C, F)=5.7 Hz), 147.59, 133.18, 123.56 (t, ³J (C, F)=9.8 Hz), 120.65, 119.72, 118.95 (t, ²J (C, F)=15.5 Hz), 112.91 (t, ⁴J (C, F)=2.3 Hz), 111.77 (dd, ²J (C, F)=16.6 Hz, ⁴J (C, F)=6.8 Hz), 109.96, 55.47.
HRMS (FAB): m/z 235.0811 (M⁺); calcd for C₁₃H₁₁F₂NO: 235.0809.
Anal. calcd (%): calcd for C₁₃H₁₁F₂NO: C66.38, H4.71, N5.95; found: C66.27, H4.53, N6.06.

The compound 33 (3.60 g, 15.3 mmol), compound 34 (5.18 g, 17.2 mmol), K₂CO₃ (4.18 g, 30.2 mmol), and copper powder (1.53 g, 24.1 mmol) were added to dry o-dichlorobenzene [ODCB] (45 ml), heated at 180° C. and stirred for 50 hours. The insoluble matter was filtered, and washed with CH₂Cl₂ (50 ml). Subsequently, water was added to the filtrate, and extracted with CH₂Cl₂ (35 ml×3). The organic layer was dried with Na₂SO₄, filtered, and the filtrate was concentrated under reduced pressure. The obtained solid was purified through silica gel column chromatography (CH₂Cl₂/hexane=1/3, Rf=0.22) to give a white solid, compound 35 (5.43 g, 13.3 mmol) at a yield of 87%.

### Mp: 57.1-58.1° C.

¹H NMR (300 MHz, CDCl₃, ppm) δ7.28 (d, ³J (H, H)=9.0 Hz, 1H), 7.14-6.99 (m, 3H), 6.95-6.80 (m, 6H), 3.61 (s, 3H), 3.58 (s, 3H).
¹³ C NMR (75 MHz, CDCl₃, ppm) δ159.00 (dd, ¹J (C, F)=249 Hz, ³J (C, F)=5.7 Hz), 154.95, 153.28, 136.88, 135.61, 125.15, 124.82, 124.62 (t, ⁴J (C, F)=9.8 Hz), 124.28 (t, ²J (C, F)=20.6 Hz), 124.27 (q, ¹J (C, F)=270 Hz), 123.1 (q, ²J (C, F)=32.1 Hz), 121.19, 120.84 (q, ³J (C, F)=4.0 Hz), 120.24 (q, ³J (C, F)=3.5 Hz), 113.09, 112.11, 111.60 (dd, ²J (C, F)=16.6 Hz, ⁴J (C, F)=6.8 Hz), 56.01, 55.91.
HRMS (FAB): m/z 409.1097 (M⁺); calcd for C₂₁ H₁₆F₅NO₂: 409.1101.
Anal. calcd (%): calcd for C₂₁H₁₆F₅NO₂: C61.62, H3.94, N3.42; found C61.76, H3.91, N3.4.

The compound 35 (4.09 g, 10.0 mmol) was dissolved in dry CH₂Cl₂ (300 ml) and cooled −78° C. BBr₃ (2.00 ml, 21.1 mmol) was added thereto and gradually heated up to room temperature, and stirred for 3 hours. The solution was put in water (100 ml), and extracted with CH₂Cl₂ (50 ml×3). This was dried with Na₂SO₄, filtered, and the filtrate was concentrated under reduced pressure to give 3.75 g a solid containing CH₂Cl₂. The obtained solid was dissolved in DMF (200 ml), then K₂CO₃ (4.15 g, 30.0 mmol) was added thereto, and stirred at 100° C. for 20 hours. The insoluble matter was filtered, and the solution was concentrated under reduced pressure. The solid was dissolved in CH₂Cl₂ (100 ml), then an aqueous 1 M NH₄Cl solution (100 ml) was added thereto and extracted with CH₂Cl₂ (70 ml×3). The organic layer was dried with Na₂SO₄, filtered, and the filtrate was concentrated under reduced pressure. The obtained solid was processed for placing point removal in silica gel chromatography (CH₂Cl₂) and then purified through silica gel chromatography (CH₂Cl₂/hexane=1/4, R_{f}=0.58) to give a white solid, compound 36 (2.20 g, 6.44 mmol) at a yield of 64%.

### Mp: 129.3-130.2° C.

¹H NMR (300 MHz, CD₂Cl₂, ppm) δ7.56 (d, ⁴J (H, H)=1.8 Hz, 1H), 7.31 (dd, ³J (H, H)=7.5 Hz, ⁴J (H, H)=2.1 Hz, 1H), 7.16 (dq, ³J (H, H)=8.4 Hz, ⁴J (H, F)=0.9 Hz, 1H), 7.04-6.90 (m, 4H), 6.80 (t, ³J (H, H)=8.1 Hz, ¹H), 6.69 (dd, ³J (H, H)=8.4 Hz, ⁴J(H, H)=0.9 Hz, 1H), 6.53 (dd, ³J(H, H)=8.1 Hz, ⁴J (H, H)=1.2 Hz, 1H).
Anal. calcd (%): calcd for C₁₉H₁₀F₃NO₂: C66.87, H2.95, N4.10; found: C66.72, H2.80, N4.07.

The compound 36 (1.72 g, 5.03 mmol) and N-bromosuccinimide (0.993 g, 5.58 mmol) were dissolved in CHCl₃ (45 ml) and acetic acid (45 ml), and stirred at room temperature for 18.5 hours. This was neutralized with an aqueous saturated solution of NaHCO₃, and extracted with CHCl₃ (50 ml×3). The organic layer was dried with Na₂SO₄, filtered, and the filtrate was concentrated under reduced pressure. The obtained solid was processed for placing point removal in silica gel chromatography (CH₂Cl₂) and then purified through silica gel chromatography (CH₂Cl₂/hexane=1/2, R_{f}=0.78) to give a white solid, compound 11e (1.90 g, 4.51 mmol) at a yield of 90%.

### Mp: 128.6-130.3° C.

¹H NMR (300 MHz, CDCl₃, ppm) δ7.48 (s, 1H), 7.1907.06 (m, 4H), 6.96 (d, ³J (H, H)=8.4 Hz, 1H), 6.80 (t, ³J (H, H)=8.1 Hz, 1H), 6.53 (d, ³J (H, H)=8.4 Hz, 2H).
HRMS (FAB): m/z 418.9770 (M⁺); calcd for C₁₉H₉BrF₃NO₂: 418.9769.
Anal. calcd (%): calcd for C₁₉H₉BrF₃NO₂: C54.31, H2.16, N3.33; found: C54.41, H2.05, N3.43.

The compound 11e (603 mg, 1.44 mmol), Ni(cod)₂ (236 mg, 0.858 mmol), 1,5-cyclooctadiene (0.23 ml, 1.87 mmol) and 2,2′-bipyridyl (270 mg, 1.73 mmol) were dissolved in dry THF (30 ml), and heated at 60° C. for 25 hours. The solution was concentrated under reduced pressure, adsorbed by silica gel using o-dichlorobenzene, and extracted with hot o-dichlorobenzene, and then concentrated under reduced pressure. The solid was washed with hexane to give a yellow solid, compound 285 (449 mg, 0.660 mmol) at a yield of 92%.

### Mp: 287.5-289.2° C.

¹H NMR (600 MHz, CD₂Cl₂, ppm) δ7.60 (d, ⁴J (H, H)=1.2 Hz, 2H), 7.37 (d, ³J (H, H)=8.4Hz, 2H), 7.23 (dd, ³J (H, H)=8.4 Hz, ⁴J (H, H)=1.8 Hz, 2H), 7.19 (dq, ³J (H, H)=8.4 Hz, ⁴J (H, F)=0.6 Hz, 2H), 7.17 (d, ⁴J (H, H)=2.4 Hz, 2H), 7.00 (dd, ³J (H, H)=8.4 Hz, ⁴J (H, H)=1.8 Hz, 2H), 6.84 (t, ³J (H, H)=7.8 Hz, 2H), 6.59 (dd, ³J (H, H)=8.4 Hz, ⁴J (H, H)=1.2 Hz, 2H), 6.56 (dd, ³J (H, H)=8.4 Hz, ⁴J (H, H)=1.2 Hz, 2H).
¹³C NMR (150 MHz, CD₂Cl₂, ppm) δ149.96, 147.56, 145.36, 145.21, 135.80, 130.14, 127.71, 126.24 (q, ²J (C, F)=33.0 Hz), 124.76, 124.27 (q, ¹J (C, F)=270 Hz), 122.02, 120.90 (q, ³J (C, F)=4.5 Hz), 120.36, 117.98, 115.62, 115.10, 112.09, 111.84 (q, ³J (C, F)=3.0 Hz), 111.60.
HRMS (FAB): m/z 680.1164 (M⁺); calcd for C₃₈H₁₈F₆N₂O₄: 680.1171.
Anal. calcd (%): calcd for C₃₈H₁₈F₆N₂O₄: C67.06, H2.67, N4.12; found: C67.30, H2.59, N4.19.

### Example 6

Compound 294 was produced according to the following scheme.

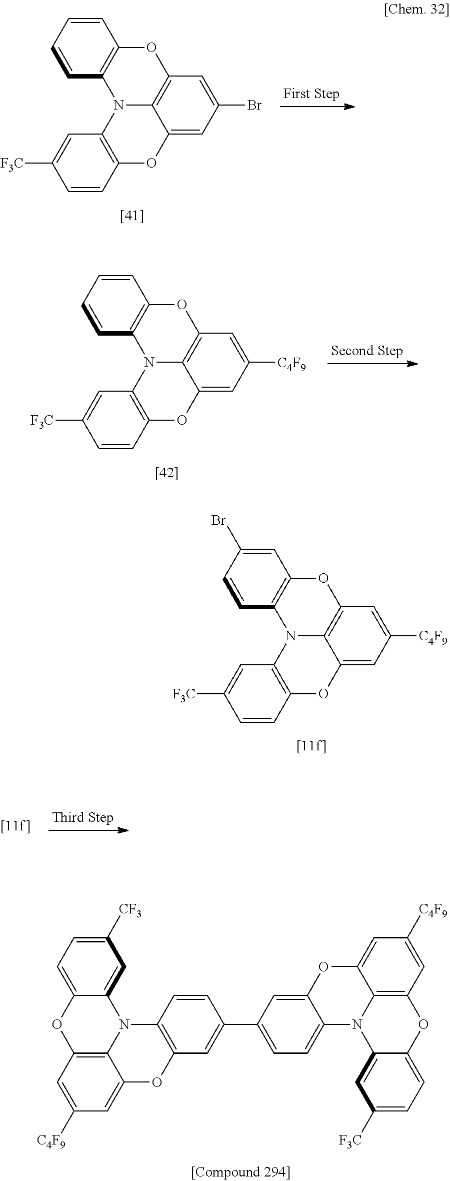

Compound 41 (121 mg, 0.288 mmol) and copper powder (56.3 mg, 0.886 mmol) were dissolved in dry dimethylsulfoxide [DMSO] (5 ml) that had been previously degassed by argon babbling (2 hours), then perfluorobutyl iodide (114 mg, 0.329 mmol) was added thereto, and heated with stirring at 110° C. for 49 hours. The insoluble matter was filtered, water (5 ml) was added, extracted with CH₂Cl₂ (20 ml×3), and washed with water. The organic layer was dried with Na₂SO₄, filtered, and the filtrate was concentrated under reduced pressure. The obtained solid was purified through silica gel column chromatography (CH₂Cl₂/hexane=1/5, Rf=0.80) to give a yellow liquid, compound 42 (112 mg, 0.200 mmol) at a yield of 69%.

¹H NMR (300 MHz, CDCl₃, ppm) δ7.55 (s, 1H), 7.29 (d, ³J (H, H)=8.1 Hz, 1H), 7.20 (d, ³J (H, H)=8.4 Hz, 1H), 7.08-6.90 (m, 4H), 6.75 ppm (d, ³J (H, H)=6.9 Hz, 2H).
HRMS (FAB): m/z 559.0449 (M⁺); calcd for C₂₃H₉F₁₂NO₂: 559.0442.

The compound 42 (106 mg, 0.190 mmol) and N-bromosuccinimide (36.4 mg, 0.204 mmol) were dissolved in CHCl₃ (5 ml) and acetic acid (5 ml), and stirred at room temperature for 14 hours. Subsequently, this was heated up to 60° C. and stirred for 6.5 hours. The reaction solution was neutralized with an aqueous saturated solution of NaHCO₃, and the aqueous layer was extracted with CHCl₃ (15 ml×3). The organic layer was dried with Na₂SO₄, filtered, and the filtrate was concentrated under reduced pressure. The obtained solid was purified through silica gel chromatography (hexane, R_{f}=0.40) to give a white solid, compound 11f (117.3 mg, 0.184 mmol) at a yield of 97%.

### Mp: 121.6-122.8° C.

¹H NMR (300 MHz, CDCl₃, ppm) δ7.48 (s, 1H), 7.23 (d, ³J (H, H)=8.7 Hz, 1H), 7.16 (s, 2H), 7.10 (s, 1H), 6.99 (d, ³J (H, H)=8.4 Hz, 1H), 6.76 ppm (s, 2H).
HRMS (FAB): m/z 638.9537 (M⁺); calcd for C₂₃H₈BrF₁₂NO₂: 638.9529.

The compound 11f (64.9 mg, 0.102 mmol), Ni(cod)₂ (17.4 mg, 0.0633 mmol), 1,5-cyclooctadiene (15 ml, 0.122 mmol) and 2,2′-bipyridyl (19.0 mg, 0.123 mmol) were dissolved in dry tetrahydrofuran [THF] (2.5 ml), and heated at 60° C. for 72 hours. The solution was concentrated under reduced pressure, adsorbed by silica gel using o-dichlorobenzene, and extracted with hot o-dichlorobenzene, and then concentrated under reduced pressure. The solid was washed with hexane to give a yellow solid, compound 294 (34.8 mg, 0.0312 mmol) at a yield of 61%.

### Mp: 246.5-248.3° C.

¹H NMR (300 MHz, CD₂Cl₂, ppm) δ7.61 (d, ⁴J (H, H)=1.5 Hz, 2H), 7.39 (d, ³J (H, H)=8.4 Hz, 2H), 7.27 (dd, ³J (H, H)=8.4 Hz, ⁴J (H, H)=2.1 Hz, 2H), 7.25 (dq, ³J (H, H)=8.1 Hz, ⁴J (H, F)=2.1 Hz, 2H), 7.19 (d, ⁴J (H, H)=2.4 Hz, 2H), 7.03 (dd, ³J (H, H)=8.4 Hz, ⁴J (H, H)=1.8 Hz, 2H), 6.80 ppm (dd, ³J (H, H)=10.2 Hz, ⁴J (H, H)=1.8 Hz, 4H).
HRMS (FAB): m/z 1116.0756 (M⁺); calcd for C₄₆H₁₆F₂₄N₂O₄: 1116.0727.
Anal. calcd (%): calcd for C₄₆H₁₆F₂₄ N₂O₄: C49.48, H1.44, N2.51; found: C49.58, H1.45, N2.74.

### Example 7

Compound 401 was produced according to the following scheme.

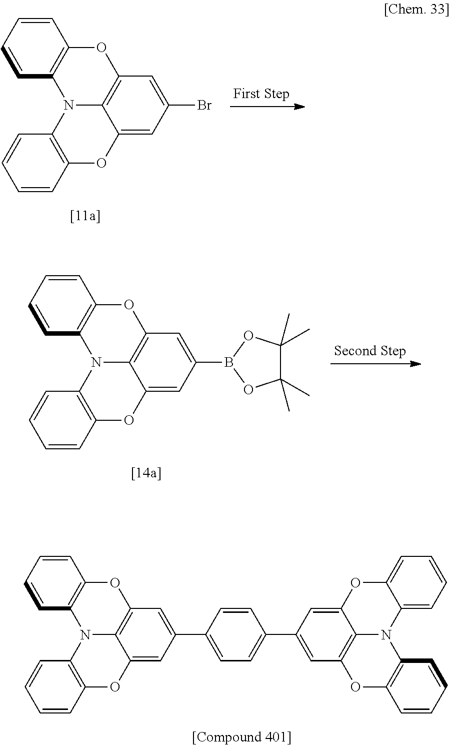

Compound 11a (1.06 g, 3.01 mmol) was dissolved in dry tetrahydrofuran [THF] (100 ml), and cooled to −78° C. N-butyl lithium (in hexane, 1.58 M, 2.0 ml, 3.16 mmol) was dropwise added thereto and stirred for 1 hour. Subsequently, 2-isopropoxy-4,4,5,5-tetramethyl-1,3,2-dioxaborolane (0.65 ml, 3.19 mmol) was added thereto, and stirred at room temperature for 5 hours. the solution was concentrated under reduced pressure, and dissolved in CH₂Cl₂ (50 ml). Water was added thereto, and the aqueous layer was extracted with CH₂Cl₂ (25 ml×3). The organic layer was dried with Na₂SO₄, filtered, and the filtrate was concentrated under reduced pressure. The obtained solid was purified through gel exclusion chromatography (toluene) to give a white solid, compound 14a (1.04 g, 2.61 mmol) at a yield of 87%.

¹H NMR (300 MHz, CDCl₃, ppm) δ7.32 (dd, ³J (H, H)=6.6 Hz, ⁴J (H, H)=2.4 Hz, 2H), 6.96-6.85 ppm (m, 8H).

Toluene and distilled water were separately degassed by argon bubbling for 4 hours. 1,4-Dibromobenzene (34.5 mg, 0.146 mmol), the compound 14a (126 mg, 0.315 mmol), Pd₂(dba)₃.CHCl₃ (4.92 mg, 0.00475 mmol), 2-dicyclohexylphosphine-2′,6′-dimethoxybiphenyl [SPhos] (7.76 mg, 0.0189 mmol) and K₃PO₄ (92.6 mg, 0.436 mmol) were put into a Schlenk flask, and purged with argon. Toluene (5 ml) and distilled water (0.5 ml) each degassed by argon bubbling (4 hours) were added thereto, and stirred at 110° C. for 39 hours. The solution was concentrated under reduced pressure, adsorbed by silica gel using o-dichlorobenzene, and extracted with hot o-dichlorobenzene, and then concentrated under reduced pressure. The solid was washed with hexane to give a yellow solid, compound 401 (38.4 mg, 0.0619 mmol) at a yield of 42%.

¹H NMR (300 MHz, 300 MHz, 1/1CD₂Cl₂/CS₂, ppm) δ7.55 (s, 4H), 7.37 (dd, ³J (H, H)=7.5 Hz, ⁴J (H, H)=1.8 Hz, 4H), 7.02-6.90 (m, 12H), 6.79 ppm (s, 4H).

### Example 8

Compound 701 was produced according to the following scheme.

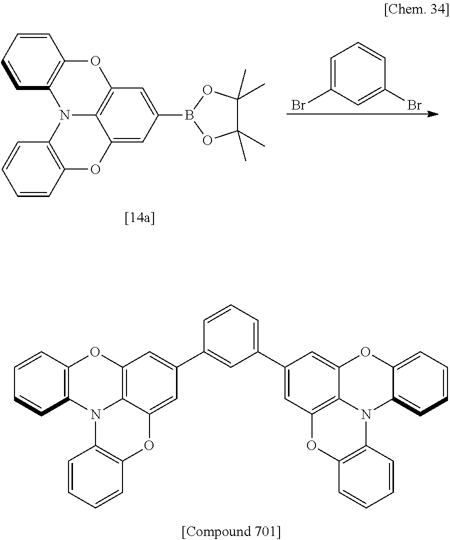

1,3-Dibromobenzene (18 μl, 0.150 mmol), the compound 14a (125 mg, 0.312 mmol), Pd₂(dba)₃.CHCl₃ (4.90 mg, 0.00473 mmol), 2-dicyclohexylphosphine-2′,6′-dimethoxybiphenyl [SPhos] (7.53 mg, 0.0183 mmol) and K₃PO₄ (96.0 mg, 0.452 mmol) were put into a Schlenk flask, and purged with argon. Toluene (6 ml) and distilled water (0.6 ml) each degasse4d by argon bubbling (2.5 hours) were added thereto, and stirred at 110° C. for 42 hours. The solution was concentrated under reduced pressure, adsorbed by silica gel using o-dichlorobenzene, and extracted with hot o-dichlorobenzene, and then concentrated under reduced pressure. The obtained solid was washed with hexane to give a pale yellow solid, compound 701 (83.9 mg, 0.135 mmol) at a yield of 90%.

¹H NMR (300 MHz, 1/1CD₂Cl₂/CS₂, ppm) δ7.62 (s, 1H), 7.46 (d, ³J (H, H)=1.2 Hz, 2H), 7.37 (dd, ³J (H, H)=6.6 Hz, ⁴J (H, H)=1.2 Hz, 4H), 7.35 (t, ³J (H, H)=1.2 Hz, 1H), 7.02-6.91 (m, 12H).

### Test Example 1

The results of cyclic voltammetry of the compound 1, the compound 2, the compound 24 and the compound 201 (dimer) obtained in Examples 1 to 4, and comparative compounds A to C (monomers) are shown in FIG. 1 and FIG. 2. The cyclic voltammetry was carried out in a CH₂Cl₂ solution, using n-Bu₄N⁺PF⁶⁻ (0.1 mol/l) as the supporting electrolyte, Ag/Ag⁺ as the reference electrode, glassy carbon as the work electrode, and Pt as the counter electrode. The results of cyclic voltammetry confirm that the compound 1, the compound 24 and the compound 201 each exhibit a two-stage reversible oxidation wave and, under the measurement condition, one corresponding radical cation and dication form stably, therefore suggesting that these compounds show excellent characteristics as a hole transport material. The compound 2 gave a reversibly-detected oxidation wave corresponding to the third and fourth stage dielectronic oxidation, in addition to the two-stage reversible oxidation wave, and it is confirmed that, under the measurement condition, the compound stably forms even the corresponding tetracation species, from which, therefore, it is suggested that the compound is an excellent hole transport material. The compound 1, the compound 2, the compound 24 and the compound 201 all have a high level of HOMO as estimated from the results of cyclic voltammetry and photoabsorption spectra, and it is confirmed that these compounds are all excellent in hole injection capability (see FIG. 3). In FIG. 3, the data of α-NPD and TPD are based on Appl. Phys. Lett., 2007, 90, 183503.

### Example 9

A thin film was formed of the compound 1 and the hole mobility thereof was measured according to the SCLC method (Appl. Phys. Lett. 2007, 90, 203512), and was from 1.2 to 2.0×10⁻⁴ cm²/Vs. FIG. 4 shows the results of the hole mobility of the compound 1, the compound 24, the compound 201 and α-NPD, as measured according to the TOF method (time-of-flight method). These results indicate that the compounds of the invention have the same level of hole mobility as that of α-NPD which is a typical hole transport material in organic electroluminescence elements. The results of the electron mobility, as measured according to the TOF method, of the compound 201 are shown in FIG. 5 along with the measurement results of the hole mobility thereof. The results show that the electron mobility of the compound 201 is further higher than the hole mobility thereof, indicating that some compounds of the invention are excellent as a bipolar material.

### Example 10

In this Example, an organic electroluminescence element (a) of the invention and a comparative organic electroluminescence element (b) were produced, as shown in FIG. 6.

The organic electroluminescence element (a) was produced by vapor-depositing the compound 1 in a thickness of 10 nm, α-NPD in a thickness of 50 nm, Alq₃ having the following structure in a thickness of 50 nm, LiF and Al in that order, on the ITO electrode of a glass substrate with an ITO electrode attached thereto (see FIG. 6(a)).

The organic electroluminescence element (b) was produced according to the same process as that for the above-mentioned organic electroluminescence element (a) except that the hole injection layer containing the compound 1 was not formed therein (see FIG. 6(b)).

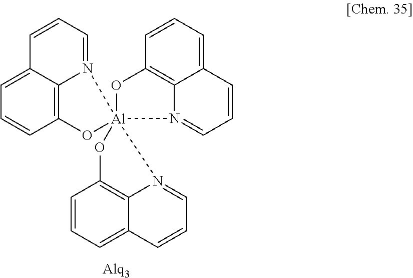

The structures of the produced organic electroluminescence element (a) and organic electroluminescence element (b) are as follows:

ITO/compound 1 (10 nm)/α-NPD (50 nm)/Alq₃ (50 nm)/LiF/Al Element (a):

ITO/α-NPD (50 nm)/Alq₃ (50 nm)/LiF/Al Element (b):

The produced organic electroluminescence element (a) and organic electroluminescence element (b) were analyzed in point of the relationship between the current density and the current efficiency thereof, and the results shown in FIG. 7 were obtained. The results confirm that using the compound 1 represented by the general formula [1] of the invention improves the current efficiency.

### Example 11

In the same manner as in Example 10, organic electroluminescence elements (c) and (d) each having the structure sheen below were produced. These organic electroluminescence elements differ from each other in point of the hole transport material therein.

ITO/compound 1 (60 nm)/Alq₃ (50 nm)/LiF/Al Element (c):

ITO/α-NPD (60nm)/Alq₃ (50 nm)/LiF/Al Element (d):

The produced organic electroluminescence elements (c) and (d) were analyzed in point of the change of the voltage and the brightness thereof for a period of 2000 hours or more at a fixed current value of 2 m/A. FIG. 8 shows the measurement results of the voltage change, and FIG. 9 shows the measurement results of the brightness change. FIG. 8 confirms that the element using the compound 1 represented by the general formula [1] of the invention suppressed the voltage increase. This indicates that use of the compound 1 makes it possible to prevent the increase in the electric resistance owing to the degradation of the element. FIG. 9 confirms that use of the compound 1 makes it possible to prevent the brightness of the element from lowering. These results indicate the effect of the compound 1 for prolonging the life of the element.

### Example 12

In the same manner as in Example 10 but using the compound 201 in place of the compound 1 used in Example 10, an organic electroluminescence elements (e) and (f) each having the structure mentioned below were produced. For comparison, an organic electroluminescence element (g) having the structure mentioned below was produced. In these organic electroluminescence elements, the total thickness of the compound 201 film and the α-NPD film was kept constant to be 60 nm. but the thickness of the compound 201 film was varied.

ITO/compound 201 (30 nm)/α-NPD (30 nm)/Alq₃ (50 nm)/LiF/Al Element (e):

ITO/compound 201 (10 nm)/α-NPD (50 nm)/Alq₃ (50 nm)/LiF/Al Element (f):

ITO/α-NPD (60 nm)/Alq₃ (50 nm)/LiF/Al Element (g):

The produced organic electroluminescence elements (e) to (g) were analyzed in point of the relationship between the current density and the current efficiency thereof, and the results shown in FIG. 10 were obtained. The results confirm that the compound 201 film formed as a hole injection layer for α-NPD increased the brightness per current, therefore confirming the increase in the current density. In addition, it is confirmed that increasing the thickness of the compound 201 film further increased the brightness per current, therefore confirming further increase in the current density. These indicate that the compound 201 is excellent also as a hole transport material.

### INDUSTRIAL APPLICABILITY

As obvious from the above, the compound represented by the general formula [1] has a stable amorphous state and hardly crystallizes and, in addition, has excellent characteristics as a charge transport material. Accordingly, using the compound represented by the general formula [1] provides an organic device such as an organic electroluminescence element, an organic thin-film solar sell and the like having high efficiency, capable of suppressing consumption power and heat generation and capable of realizing long-life operation. Therefore, the invention has high-level industrial applicability.

### REFERENCE SIGNS LIST

### 1 ITO Electrode-Having Glass Substrate

### 2 Compound 1

### 3 α-NPD

### 4 Alq₃

### 5 LiF

6 Al

1. A compound represented by the following general formula [1]: 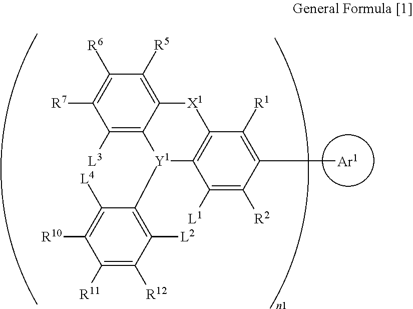
wherein Ar¹ represents a single bond
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom;
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom and a silicon atom; the other of L¹ and L², and L³ and L⁴ each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates 2, n1's X¹, Y¹, R¹, R², R⁵ to R⁷ and R¹⁰ to R¹² existing in the molecule may be the same or different;
the adjacent two R¹'s may bond to each other to form a linking group, and the adjacent two R²'s may bond to each other to form a linking group.

2. The compound according to claim 1, wherein in the general formula [1], the linking group formed by either one of L¹ and L², and L³ and L⁴, and the linking group represented by X¹ each are independently —O—, —S—, —SO₂—, >CR²¹R²², >C═O, >C═CR²³R²⁴, >C═NR²⁵, >NR²⁶, 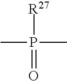
or >SiR²⁸R²⁹;
Y¹ is >N—, >B—, >P— or >P(═O)—;
R¹, R², R²¹, R²², R²⁸ and R²⁹ each are independently a hydrogen atom, a substituted or unsubstituted alkyl group, or a substituted or unsubstituted alkoxy group;
R⁵ to R⁷ and R¹⁰ to R¹² each are independently a hydrogen atom, a substituted or unsubstituted alkyl group, a substituted or unsubstituted alkoxy group, a substituted or unsubstituted aryl group, or a substituted or unsubstituted aryloxy group, or R⁵ and R⁶, R⁶ and R⁷, R¹⁰ and R¹¹, and R¹¹ and R¹² bond to each other to form a linking group; and
R²³ to R²⁷ each are independently a hydrogen atom, a substituted or unsubstituted alkyl group, or a substituted or unsubstituted aryl group.

3. The compound according to claim 1, wherein in the general formula [1], the linking group formed by either one of L¹ and L², and L³ and L⁴, and the linking group represented by X¹ each are —O—.

4. The compound according to claim 1, wherein in the general formula [1], Y¹ is >N—.

5. The compound according to claim 1, wherein in the general formula [1], R¹ and R² each are a hydrogen atom.

6. The compound according to claim 1, wherein in the general formula [1], R⁵, R⁷, R¹⁰ and R¹² each are a hydrogen atom, and R⁶ and R¹¹ each are a hydrogen atom or an alkoxy group.

7. The compound according to claim 1, wherein the molecule is asymmetric.

8. A charge transport material comprising a compound represented by the following general formula [1]:
General Formula [1] 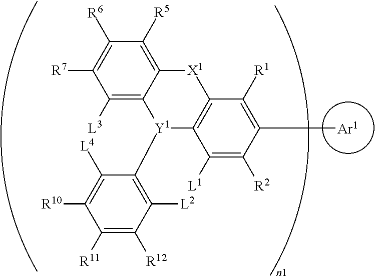
wherein Ar¹ represents a single bond
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom;
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom, and a silicon atom; the other of L¹ and L², and L³ and L⁴ each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates 2, and n1's X¹, Y¹, R¹, R², R⁵ to R⁷ and R¹⁰ to R¹² existing in the molecule may be the same or different;
the adjacent two R¹'s may bond to each other to form a linking group, and the adjacent two R²'s may bond to each other to form a linking group.

9. An organic device using a compound represented by the following general formula [1]:
General Formula [1] 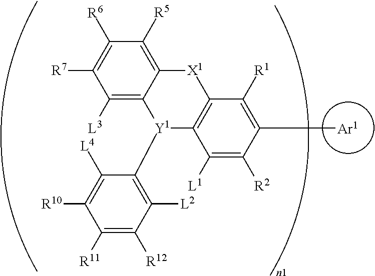
wherein Ar¹ represents a single bond
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom:
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom, and a silicon atom; the other of L¹ and L², and L³ and L⁴ each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates 2, and n1's X¹, Y¹, R¹, R², R⁵ to R⁷ and R¹⁰ to R¹² existing in the molecule may be the same or different;
the adjacent two R¹'s may bond to each other to form a linking group, and the adjacent two R²'s may bond to each other to form a linking group.

10. An electroluminescence element using a compound represented by the following general formula [1]:
General Formula [1] 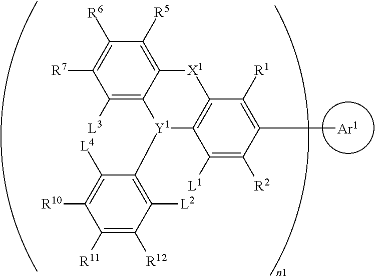
wherein Ar¹ represents a single bond
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom;
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom, and a silicon atom; the other of L¹ and L², and L³ and L⁴ each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates 2, and n1's X¹, Y¹, R¹, R², R⁵ to R⁷ and R¹⁰ to R¹² existing in the molecule may be the same or different;
the adjacent two R¹'s may bond to each other to form a linking group, and the adjacent two R²'s may bond to each other to form a linking group.

11. A photoelectric conversion element using a compound represented by the following general formula [1]:
General Formula [1] 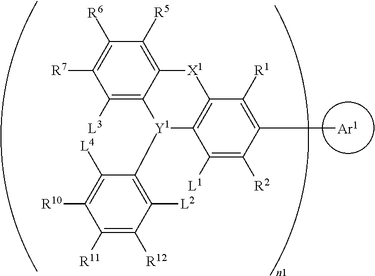
wherein Ar¹ represents a single bond
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom;
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom, and a silicon atom; the other of L¹ and L², and L³ and L^{b 4} each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates 2, and n1's X¹, Y¹, R¹, R², R⁵ to R⁷ and R¹⁰ to R¹² existing in the molecule may be the same or different;
the adjacent two R¹'s may bond to each other to form a linking group, and the adjacent two R²'s may bond to each other to form a linking group.

12. An organic thin-film solar cell using a compound represented by the following general formula [1]:
General Formula [1] 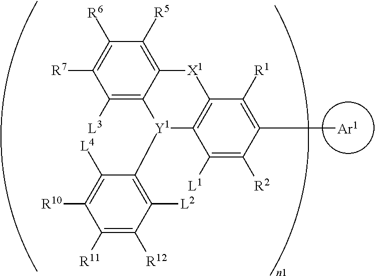
wherein Ar¹ represents a single bond
X¹ represents a linking group that links to the formula via one atom selected from a group consisting of an oxygen atom, a sulfur atom, a carbon atom, a nitrogen atom, a phosphorus atom and a silicon atom;
Y¹ represents a linking group that links to the formula via one atom selected from a group consisting of a nitrogen atom, a boron atom and a phosphorus atom;
either one of L¹ and L², and L³ and L⁴ bond to each other to represent a linking group that links to the formula via one atom selected from a group consisting of an oxygen atom, a sulfur atom, a carbon atom, a nitrogen atom, a phosphorus atom, and a silicon atom; the other of L¹ and L², and L³ and L⁴ each independently represent a hydrogen atom or a substituent;
R¹, R², R⁵ to R⁷ and R¹⁰ to R¹² each independently represent a hydrogen atom or a substituent; R⁵ and R⁶, R⁶ and R⁷, R¹⁰ and R¹¹, R¹¹ and R¹² may bond to each other to form a linking group;
n1 indicates 2, and n1's X¹, Y¹, R¹, R², R⁵ to R⁷ and R¹⁰ to R¹² existing in the molecule may be the same or different;
the adjacent two R¹'s may bond to each other to form a linking group, and the adjacent two R²'s may bond to each other to form a linking group.
